(19) United States
(12) Patent Application Publication
(10) Pub. No.: US 2013/0189354 A1
(43) Pub. Date: Jul. 25, 2013

(54) Novel Pharmaceutical Compositions for Treating Chronic Pain and Pain Associated with Neuropathy

(71) Applicant: Chandra U. Singh, San Antonio, TX (US)

(72) Inventor: Chandra U. Singh, San Antonio, TX (US)

(73) Assignee: Trinity Laboratories, Inc.,, San Antonio, TX (US)

(21) Appl. No.: 13/877,980

(22) PCT Filed: Oct. 7, 2011
(86) PCT No.: PCT/US11/55477

§ 371 Date: Apr. 5, 2013

Related U.S. Application Data
(60) Provisional application No. 61/390,981, filed on Oct. 7, 2010.

(51) Int. Cl. A61K 33/06; A61K 31/231; A61K 31/197; A61K 31/135; A61K 31/195

(52) U.S. Cl. CPC A61K 33/06; A61K 31/135; A61K 31/195; A61K 31/197; A61K 31/231
USPC 424/451; 424/682; 424/490

(57) ABSTRACT

The present invention relates to compositions and methods for treating pain wherein the compositions comprise a combination of tramadol or a pharmaceutically acceptable salt thereof, magnesium or a pharmaceutically acceptable salt thereof; and gabapentin or pregabalin. The therapeutic combination can further contain capsaicin or an ester of capsaicin.

### BACKGROUND

1. Field of the Invention

The field of the invention relates to compositions for treating pain, and in particular, pain associated with neuropathy.

2. Description of Related Art

Chronic pain is a common problem that presents a major challenge to healthcare providers because of its complex natural history, unclear etiology, and poor response to therapy. Chronic pain is a poorly defined condition. Most authors consider ongoing pain lasting longer than 6 months as diagnostic, and others have used 3 months as the minimum criterion. In chronic pain, the duration parameter is used arbitrarily. Some authors suggest that any pain that persists longer than the reasonable expected healing time for the involved tissues should be considered chronic pain. The pathophysiology of chronic pain is multifactorial and complex and still is poorly understood. Some authors have even suggested that chronic pain might be a learned behavioral syndrome that begins with a noxious stimulus that causes pain. Patients with several psychological syndromes (e.g., major depression, somatization disorder, hypochondriasis, conversion disorder) are prone to developing chronic pain. Pain is the most common complaint that leads patients to seek medical care. Chronic pain is not uncommon. Approximately 35% of Americans have some element of chronic pain, and approximately 50 million Americans are disabled partially or totally due to chronic pain.

Various neuromuscular, reproductive, gastrointestinal, and urologic disorders may cause or contribute to chronic pain. Sometimes multiple contributing factors may be present in a single patient. Chronic pain can result from musculoskeletal disorders such as osteoarthritis/degenerative joint disease/spondylosis, rheumatoid arthritis, lyme disease, reiter syndrome, disk herniation/facet osteoarthropathy, fractures/compression fracture of lumbar vertebrae, faulty or poor posture, fibromyalgia, polymyalgia rheumatica, mechanical low back pain, chronic coccygeal pain, muscular strains and sprains, pelvic floor myalgia (levator ani spasm), piriformis syndrome, rectus tendon strain, hernias (e.g. obturator, sciatic, inguinal, femoral, spigelian, perineal, umbilical), abdominal wall myofascial pain (trigger points), chronic overuse syndromes (e.g., tendinitis, bursitis); neurological disorders such as, brachial plexus traction injury, cervical radiculopathy, thoracic outlet syndrome, spinal stenosis, arachnoiditis syndrome, metabolic deficiency myalgias, polymyositis, neoplasia of spinal cord or sacral nerve, cutaneous nerve entrapment in surgical scar, postherpetic neuralgia (shingles), neuralgia (e.g., iliohypogastric, ilioinguinal, or genitofemoral nerves), polyneuropathies, polyradiculoneuropathies, mononeuritis multiplex, chronic daily headaches, muscle tension headaches, migraine headaches, temporomandibular joint dysfunction, temporalis tendonitis, sinusitis, atypical facial pain, trigeminal neuralgia, glossopharyngeal neuralgia, nervus intermedius neuralgia, sphenopalatine neuralgia, referred dental or temporomandibular joint pain, abdominal epilepsy, abdominal migraine, urologic disorders, bladder neoplasm, chronic urinary tract infection, interstitial cystitis, radiation cystitis, recurrent cystitis, recurrent urethritis, urolithiasis, uninhibited bladder contractions (detrusor-sphincter dyssynergia), urethral diverticulum, chronic urethral syndrome, urethral carbuncle, prostatitis, urethral stricture, testicular torsion, peyronie disease; gastrointestinal disorders such as chronic visceral pain syndrome, gastroesophageal reflux, peptic ulcer disease, pancreatitis, chronic intermittent bowel obstruction, colitis, chronic constipation, diverticular disease, inflammatory bowel disease, irritable bowel syndrome; reproductive disorders (extrauterine) such as endometriosis, adhesions, adnexal cysts, chronic ectopic pregnancy, chlamydial endometritis or salpingitis, endosalpingiosis, ovarian retention syndrome (residual ovary syndrome), ovarian remnant syndrome, ovarian dystrophy or ovulatory pain, pelvic congestion syndrome, postoperative peritoneal cysts, residual accessory ovary, subacute salpingo-oophoritis, tuberculous salpingitis; reproductive disorders (uterine) such as adenomyosis, chronic endometritis, atypical dysmenorrhea or ovulatory pain, cervical stenosis, endometrial or cervical polyps, leiomyomata, symptomatic pelvic relaxation (genital prolapse), intrauterine contraceptive device; psychological disorders such as bipolar personality disorders, depression, porphyria, sleep disturbances; and other conditions such as cardiovascular disease (eg, angina), peripheral vascular disease and chemotherapeutic, radiation, or surgical complications.

The modern concept of pain treatment emphasizes the significance of prophylactic prevention of pain, as pain is more easily prevented than it is relieved. Pain is generally controlled by the administration of short acting analgesic agents, steroids and non-steroidal anti-inflammatory drugs. Analgesic agents include opiates, agonistic-antagonistic agents, and anti-inflammatory agents.

Opiates, a class of centrally acting compounds, are the most frequently used agents for pain control. Opiates are narcotic agonistic analgesics and are drugs derived from opium, such as morphine, codeine, and many synthetic congeners of morphine, with morphine and hydrocodone preparations being the most widely used opiates. Opiates are natural and synthetic drugs with morphine-like actions. Opiates are narcotic agonistic analgesics which produce drug dependence of the morphine type and are subject to control under Federal narcotics law and the laws of most other nations and international organizations because of their addicting properties and the subsequent destructive toll exacted on the abusers and those with any connection to them. The term “opiates” also includes opiate antagonists that are essentially devoid of agonist activity at any opiate receptor, partial agonists, and opiates with mixed actions, that is they are mixed function agonist-antagonists, which are agonists at some receptors and antagonists at other receptors.

The chemical classes of opiates with morphine like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives. The principal phenanthrenes are morphine, codeine, and thebaine. The principal benzoisoquinolines are papaverine, a smooth muscle relaxant, and noscapine. Semi-synthetic derivatives of morphine include diacetylmorphine (heroin), hydromorphone, oxymorphone, hydrocodone, apomorphine, etorpine, and oxycodone. Phenylpiperidine derivatives include meperidine and its congeners diphenoxylate and loperamide, alphaprodine, anileridine hydrochloride or phosphate, and piminodine mesylate. The currently used morphinan derivative is levorphanol. The diphenyl-heptane derivatives include methadone and its congeners, and propoxyphene. Propionanilide derivatives include fentanyl citrate and its congeners sufentanil citrate and alfentanil hydrochloride. These opiate analgesics are discussed in detail in Goodman and Gilman's The Pharmacological Basis of Therapeutics, Chapter 21, “Opiate Analgesics and Antagonists”, pp. 485-521 (8^{th} ed. 1990), which is incorporated herein by reference.

The potency of all opiates is roughly comparable and can be effective against the most severe pain with appropriate dosing at intervals. In addition to the μ-opiate receptor agonists such as morphine, other classes of analgesic agents that are commonly used include agonistic-antagonistic analgesic agents, non-steroidal anti-inflammatory drugs (NSAIDS), steroids, cyclooxygenase inhibitors, anti-depressants, minerals such as magnesium, tryptan drugs for migraines, ergotamine and related compounds for migrainous headache and dissociative psychoactive drugs. Agonistic-antagonistic analgesic agents are effective for the alleviation of moderate to severe pain, but due to their antagonistic properties, their analgesic efficacy does not increase by increasing the dosage above a certain level.

However, all opiates have a wide variety of side effects that can decrease their clinical utility in certain situations. The side effects associated with the use of opiates include respiratory depression, reduced cough reflex, bronchial spasms, nausea, vomiting, release of histamine, peripheral vasodilation, orthostatic hypotension, alteration of vagal nerve activity of the heart, hyperexcitability of smooth muscles (sphincters), reduction of peristaltic motility in the gastrointestinal tract and urinary retention. Opiates also stimulate the release of adrenaline, anti-diuretic hormone, cause changes in the regulation of body temperature and sleep pattern, and are liable to promote the development of tolerance and addiction.

Furthermore, higher doses of agonistic-antagonistic analgesic agents are often associated with unpleasant sympathomimetic side effects such as tachycardia, increase in blood pressure, seizure and psychotomimetic effects such as drug induced psychosis, hyper-aggressive behavior and agitation. However, the risk of respiratory depression also decreases proportionately with the diminished analgesic activity of the higher doses. Agonistic-antagonistic analgesic agents with pharmacological activity similar to the morphine like opiates include pentazocine, nalbuphine, butorphanol, nalorphine, buprenorphine (a partial agonist), meptazinol, dezocine, and cyclazocine.

The NSAIDs include the salicylates such as salicylamide and acetylsalicylic acid (aspirin). Non-aspirin NSAIDs include para-aminophenol derivatives such as phenacetin, the pyrazole derivatives such as antipyrine, aminopyrine, dypyrone, nefenamic acid, indomethacin, methimazole, paracetamol, diclophenac sodium/potassium, ibuprofen, naproxen, and ketorolac tromethamine, all of which can be combined with opiates or used alone to alleviate milder pain. The mechanism of action of NSAIDs is by direct action at the site of tissue injury. NSAIDs peripherally inhibit cyclooxygenases (COX), the enzymes responsible for providing an activated substrate molecules for the synthesis of prostaglandins, which are a group of short-acting mediators of inflammation. The maximal analgesic effect of a standard 325 mg dose of aspirin or of NSAIDs is adjusted to provide the level of pain relief comparable to that achieved by the administration of five milligrams of morphine administered intramuscularly. The analgesic acetaminophen is often categorized as a NSAID even though the compound does not exhibit significant anti-inflammatory activity. Unless otherwise indicated, acetaminophen will be referred to herein as a NSAID.

Another difficulty that has recently been gaining increasing attention is the negative side effects of non-steroidal anti-inflammatory agents. Side effects of NSAIDs include gastrointestinal irritation, clotting difficulty and secondary anemia, bronchospastic effects in asthmatic mammalian patients, and tinnitus. The overuse of NSAIDS is in fact largely due to the inappropriate under-treatment of pain in individuals who for whatever reason do not use more effective drugs that operate on other parts of the pain pathway.

The analgesic agents are all used in similar ways to treat chronic pain in humans. However, humans will develop tolerance to the analgesic effect and develop psychological and physical dependencies on these agents, especially the opiates, thereby reducing the effectiveness of the pain treatment and exacerbating the suffering of the patient. The long term administration of narcotic analgesics to patients suffering from various types of chronic pain such as causalgia, hyperesthesia, sympathetic dystrophy, phantom limb syndrome, denervation, etc., is subject to a number of serious drawbacks including the development of opiate tolerance and/or dependence, severe constipation, and so forth.

Physical dependence or drug addiction to narcotic drugs has been traditionally treated by drug withdrawal through withholding the opiate from the drug dependent individual, gradually decreasing the amount of opiate taken by the individual, administering an opiate antagonistic drug, or substituting another drug, such as methadone, buprenorphine, or methadyl acetate for the opiate to ameliorate the physical need for the opiate. In addition the psychology of the person is treated through therapeutic interventions such as individual and group therapies. When an opiate is discontinued withdrawal symptoms appear. The character and severity of the withdrawal symptoms are dependent upon such factors as the particular opiate being withdrawn, the daily dose of the opiate, the duration of use of the opiate and the health of the drug dependent individual. The physical and psychological pain associated withdrawal symptoms can be quite severe.

An alternative approach to pain treatment employing the analgesic agents described above was tried in which an aromatic amino acid, tryptophan, was administered to persons undergoing third molar surgery to alleviate the pain and reduce or eliminate the consumption of other analgesics. The rationale was that serotonin, a neurotransmitter and a component of the serotonergic pain suppression pathway, is synthesized from tryptophan after the tryptophan is transported across the blood-brain barrier. Tryptophan is a precursor of serotonin and it was assumed that it would have analgesic effects. It was found however that tryptophan had no effect on post-operative pain or on the consumption of other analgesics (Ekblom, A., et al, “Tryptophan supplementation does not affect post-operative pain intensity or consumption of analgesics” Pain 1991; 44:249-254).

Other treatments include the use of antidepressants, specifically, the tricyclic antidepressants (TCA's), such as amytriptiline. These relieve pain by altering levels of serotonin in the body. The antineuralgic properties of TCA's were shown to be independent from their antidepressant properties. TCA's are associated with a number of adverse side effects such as sedation, orthostatic hypotension, dry mouth, urinary retention, constipation, and weight gain. These side effects are more pronounced in the elderly. TCA's should be used with caution in the elderly, patients with heart disease, narrow angle glaucoma, and prostatism. Another class of antidepressants, the selective serotonin reuptake inhibitors (SSRI's), may also be used. In general, the SSRI's have not been found to be as effective as the TCA's for the treatment of neuroptahic pain, but are better tolerated. The side effects of the SSRI's include sweating, stomach upset, somnolence, dizziness, decreased libido, and ejaculatory disturbances.

Changes in serotonin transport function and in neuroreceptor loading that occur over the course of antidepressant use create a dependence on the drug that takes some time to be eliminated even when the drug is no longer needed to stabilize depression. Adverse effects that can arise from reducing the drug dose have been given a name: SSRI Withdrawal Syndrome or SSRI Discontinuation Syndrome (Bull S A, et al., Discontinuing or switching selective serotonin-reuptake inhibitors, Annals of Pharmacotherapy 2002; 36(4): 578-584; Barbui C, et al., Selective serotonin reuptake inhibitors versus tricyclic and heterocyclic antidepressants: comparison of drug adherence, In: The Cochrane Library, Issue 4, 2000. Oxford). To avoid this syndrome, very gradual withdrawal—as little as 5% dosage decline per week has been recommended; rarely are the drugs withdrawn at a rate of more than 20% per week. Unfortunately, many patients are hesitant to spend this much time withdrawing from the drug, and many physicians do not recommend such gradual dosage decline, believing that the majority of the patients will do well with relatively rapid withdrawal, so SSRI Withdrawal Syndrome can readily occur; some patients may experience the symptoms even with very gradual tapering of dosage.

U.S. Pat. No. 5,578,645 teaches the method for treating acute or chronic pain in a mammal comprising the administration of a therapeutically effective amount of an analgesic solution composed of at least one branched chain amino acid selected from the group consisting of leucine, isoleucine, and valine, or administering a therapeutically effective amount of an analgesic solution comprising an analgesic agent selected from the group consisting of an opiate, an agonistic-antagonistic agent, and an anti-inflammatory agent, and at least one branched chain amino acid selected from the group consisting of leucine, isoleucine, and valine.

U.S. Pat. No. 4,769,372 describes a method for treating chronic pain or chronic cough in a patient while preventing or alleviating the development of constipation or other symptoms of intestinal hypomotility wherein an opiate analgesic or antitussive such as morphine, meperidine, oxycodone, hydromorphone, codeine and hydrocodone is administered to the patient together with an opiate antagonist such as naloxone, naloxone glucuronide or nalmefene glucuronide. However successful this therapeutic combination may be in inhibiting the development of constipation or other symptoms of intestinal hypomotility, it does not address the problems of tolerance and/or dependence that are associated with the long term administration of narcotic analgesics.

Other approaches to the treatment of chronic pain and neuropathic pain have included the administration of a pharmaceutically acceptable acid addition salt or a protonated derivative of at least one microtubule inhibitor such as vinblastine, dexacetoxyvinblastine, vincristine, vindesine, leurosine and N-formyl-leurosine as disclosed in U.S. Pat. No. 4,602,909, (3S,4S)-7-hydroxy-Δ⁶-tetrahydro-cannabinol homologues and derivatives essentially free of the (3R,4R) form as disclosed in Hayes et al, Pain, 48 (1992) 391-396, Mao et al, Brain Res., 584 (1992) 18-27, 584 (1992) 28-35 and 588 (1992) 144-149 and the N-methyl-D-aspartate (NMDA) receptor antagonist, or blocker, MK801 (the compound 5-methyl-10, 11-dihydro-5H-dibenzo[a,d]cyclohepten-5,10-imine) as disclosed in Mao et al, Brain Res., 576 (1992) 254-262. It was noted that MK 801 was unsuitable for use as a therapeutic due to its pronounced central nervous system neurotoxicity.

Dextromethorphan (frequently abbreviated as DM) is the common name for (+)-3-methoxy-N-methylmorphinan (FIG. 1). It is widely used as a cough suppressant, and is described in references such as Rodd (Rodd E H. Chemistry of Carbon Compounds, Elsevier Publ, New York, 1960) and Goodman and Gilman's Pharmacological Basis of Therapeutics (Brunton L L, Blumenthal D K, Murri N, Dandan R H, Knollmann B C. Goodman & Gilman's The Pharmacological Basis of Therapeutics. 12th ed. New York: McGraw-Hill, 2011. ISBN 13:978-0-07-1624428). Briefly, DM is a non-addictive opiate comprising a dextrorotatory enantiomer (mirror image) of the morphinan ring structure that forms the molecular core of most opiates. DM acts at a class of neuronal receptors known as sigma (a) receptors. These are often referred to as μ opiate receptors, but there is some question as to whether they are opiate receptors, so many researchers refer to them simply as μ receptors, or as high-affinity dextromethorphan receptors. They are inhibitory receptors, which mean that their activation by DM or other μ-agonists causes the suppression of certain types of nerve signals. Dextromethorphan also acts at another class of receptors known as N-methyl-D-aspartate (NMDA) receptors, which are one type of excitatory amino acid (EAA) receptor. Unlike its agonist activity at μ receptors, DM acts as an antagonist at NMDA receptors, which means that DM suppresses the transmission of nerve impulses mediated by NMDA receptors. Since NMDA receptors are excitatory receptors, the activity of DM as a NMDA antagonist also leads to the suppression of certain types of nerve signals, which may also be involved in some types of coughing. Due to its activity as a NMDA antagonist, DM and one of its metabolites, dextrorphan, are being actively evaluated as possible treatments for certain types of excitotoxic brain damage caused by ischemia (low blood flow) and hypoxia (inadequate oxygen supply), which are caused by events such as stroke, cardiac arrest, and asphyxia. The anti-excitotoxic activity of dextromethorphan and dextrorphan, and the blockade of NMDA receptors by these drugs, are discussed in items such as Choi (Choi D W. Dextrorphan and dextromethorphan attenuate glutamate neurotoxicity. Brain Res 1987; 403: 333-6.), Wong et al (Wong B Y, Coulter D A, Choi D W, Prince D A. Dextrorphan and dextromethorphan, common antitussives, are antiepileptic and antagonize N-methyl-D-aspartate in brain slices. Neurosci Lett. 1988 Feb. 29; 85(2):261-266.) and Steinberg et al (Steinberg G K et al, Delayed treatment with dextromethorphan and dextrorphan reduces cerebral damage after transient focal ischemia, Neurosci Letters 1988; 89: 193-197) and U.S. Pat. No. 4,806,543. Dextromethorphan has also been reported to suppress activity at neuronal calcium channels (Carpenter C L et al, Dextromethorphan and dextrorphan as calcium channel antagonists, Brain Research 1988; 439: 372-375). Dextromethorphan and the receptors it interacts with are further discussed in Tortella et al (Tortella F C, Pellicano M, Bowery N G. Dextromethorphan and neuromodulation: old drug coughs up new activities. Trends Pharmacol Sci. 1989 December; 10(12):501-507), Leander et al (Leander J D, Rathbun R C, Zimmerman D M. Anticonvulsant effects of phencyclidine-like drugs: relation to N-methyl-D-aspartic acid antagonism. Brain Res. 1988 Jun. 28; 454(1-2):368-372), Koyuncuoglu & Saydam (Koyuncuoglu and Saydam. Intnl J Clin Pharmacol Ther Tox 1990; 28: 147-152), Ferkany et al (Ferkany J W, Borosky S A, Clissold D B, Pontecorvo M J. Dextromethorphan inhibits NMDA-induced convulsions. Eur J Pharmacol. 1988 Jun. 22; 151(1):151-154), Prince & Feeser (Prince & Feeser. Neurosci Letters 1988; 85: 291-296) and Musacchio et al (Musacchio J M et al, High affinity dextromethorphan binding sites in the guinea pig brain, J Pharmacol Exp Ther 1988; 247: 424-431).

DM disappears fairly rapidly from the bloodstream (see, e.g., Vettican S J et al, Phenotypic differences in dextromethorphan metabolism, Pharmaceut Res 1989; 6: 13-19). DM is converted in the liver to two metabolites called dextrorphan and 3-methoxymorphinan, by an enzymatic process called O-demethylation; in this process, one of the two pendant methyl groups is replaced by hydrogen. If the second methyl group is removed, the resulting metabolite is called 5-hydroxymorphinan. Dextrorphan and 5-hydroxymorphinan are covalently bonded to other compounds in the liver (primarily glucuronic acid or sulfur-containing compounds such as glutathione) to form glucuronide or sulfate conjugates which are eliminated fairly quickly from the body via urine bloodstream.

Dextrorphan, the major metabolite of the anti-tussive dextromethorphan, and ketamine, are known NMDA receptor antagonists. Unlike MK 801 they have few, if any, neurotoxic side effects. U.S. Pat. No. 5,352,683 discloses a method for the alleviation of chronic pain in a mammal suffering there from by administration of a nontoxic N-methyl-D-aspartate receptor antagonist such as dextromethorphan, dextrorphan, ketamine or pharmaceutically acceptable salt thereof, alone or in combination with a local anesthetic and optionally in sustained release dosage form.

In summary, Dextromethorphan and its active metabolite dextrorphan bind to the N-Methyl-D-Aspartate (NMDA) glutamate and nicotine/neuronal nicotinic receptors as inhibitors. Dextromethorphan and dextrorphan also bind to the receptor-gated (NMDA receptor mediated) and voltage-gated calcium channels, and the voltage-gated sodium channels as a blocker. Through these bindings, dextromethorphan and dextrorphan modulates the glutamate pathway in the central nervous system (CNS) and modulate most of the excitatory synaptic transmission. Dextromethorphan and dextrorphan also bind to the sigma receptors which are found in high concentrations in limbic and motor areas of the CNS sensory processing such as the dorsal root ganglia and the nucleus tractus solitarus (NTS). In addition, Dextromethorphan inhibits the reuptake of 5-HT (serotonin) and norepinephrine, thus modulating the monamine pathways.

Tramadol has the chemical name (+/−)-trans (RR,SS)-2-[(di-methylamino)methyl]-1-(3-methoxyphenyl)cyclohexanol, and which is often erroneously referred to in literature as the cis(RS,SR) diastereomer. Tramadol is a centrally acting, binary analgesic that is neither opiate-derived, nor is it an NSAID. It is used to control moderate pain in chronic pain settings, such as osteoarthritis and post-operative analgesia, and acute pain, such as dental pain.

Tramadol is a racemate and consists of equal quantities of (+)- and (−)-enantiomers (FIG. 1). It is known that the pure enantiomers of tramadol have a differing pharmaceutical profiles and effects when compared to the racemate. The (+)-enantiomer is distinguished by an opiate-like analgesic action due its binding with the μ-opiate receptor, and both enantiomers inhibit 5-hydroxytryptamine (serotonin) and noradrenaline (norepinephrine) reuptake, which is stronger than that of racemic mixtures of tramadol, while distinct inhibition of noradrenaline reuptake is observed with the (−)-enantiomer. It has been proven for (+)- and (−)-tramadol that, depending upon the model, the two enantiomers mutually reinforce and enhance their individual actions (Raffa R B, Friderichs E, Reimann W, Shank R P, Codd E E, Vaught J L, Jacoby H I, Selve N. Complementary and synergistic antinociceptive interaction between the enantiomers of tramadol J Pharmacol Exp Ther 1993; 267: 331-40; Wiebalck A et al, “Sind Tramadol-Enantiomere für die postoperative Schmerztherapie besser geeignet als das Racemat? Eine randomisierte, Plazebo- und Morphin-kontrollierte Doppelblindstudie”, Der Anaesthesist, 1998; 47: 387-394). It is obvious to conclude that the potent analgesic action of tramadol is based on this mutually dependent reinforcement of action of the enantiomers. Tramadol's major active metabolite, O-desmethyltramadol (M1), shows higher affinity for the □1-opiate receptor and has at least twice the analgesic potency of the parent drug. O-desmethyl-N-mono-desmethyltramadol (referred to as M5 in some places in the following text and in the literature) is known as one of the in vivo metabolites of tramadol (1RS, 2RS)-2[(dimethylamino)methyl]-1-(3-methoxyphenyl)cyclohexanol (Lintz et al, Arzneim.-Forsch./Drug Res. 1981; 31(11): 1932-1943). M5 penetrates the blood-brain barrier to only a limited extent, as the effects on the central nervous system, for example analgesic effects, are distinctly less pronounced on intravenous administration than on intracerebroventricular administration. Despite the fact that tramadol is chemically unrelated to the opiates adverse side effects associated with administration of tramadol are similar to those of the opiates.

Unlugenc et al, (Unlugenc H, Gunduz M, Ozalevli M, Akman H. A comparative study on the analgesic effect of tramadol, tramadol plus magnesium, and tramadol plus ketamine for postoperative pain management after major abdominal surgery. Acta anaesthesiologica Scandinavica 2002; 46:1025-30) have shown that adding magnesium or ketamine to tramadol improved analgesia and patient comfort and decreased the amount of tramadol required for postoperative pain management after major abdominal surgery. Chen et al, (Chen Yong, Chan Sui Y, Ho Paul C. Isobolographic analysis of the analgesic interactions between ketamine and tramadol. Journal of pharmacy and pharmacology 2002; 54:623-31) have shown that in the acute thermal or chemical pain model, ketamine is not effective and the net effect of ketamine and tramadol in combination was simply additive after systemic administration. However, the co-administration produced synergistic antinociception in the chemical-induced persistent pain model.

Venlafaxine is a novel SSRI chemically unrelated to other SSRIs but chemically similar to the tramadol (FIG. 1; Markowitz J S, Patrick K S. Venlafaxine-tramadol similarities. Medical Hypotheses 1998; 51:167-8). The chemical structures of venlafaxine and tramadol are similar, demonstrating the similarity between these two antidepressant and analgesic substances, respectively. It is designated (R/S)-1-[2-(dimethylamino)-1-(4-methoxyphenyl)ethyl]cyclohexanol or (±)-1-[a-[(dimethylamino)methyl]-p-methoxybenzyl]cyclohexanol and has the empirical formula of C₁₇H₂₇NO₂. Venlafaxine hydrochloride is a white to off-white crystalline solid with a solubility of 572 mg/mL in water (adjusted to ionic strength of 0.2 M with sodium chloride. Venlafaxine hydrochloride (Effexor) is formulated as capsule for oral administration. Capsules contain venlafaxine hydrochloride equivalent to 37.5 mg, 75 mg, or 150 mg venlafaxine.

The mechanism of the antidepressant action of venlafaxine in humans is believed to be the same as with other SSRIs, associated with its potentiation of neurotransmitter activity in the CNS as with other SSRIs: preclinical studies have shown that venlafaxine and its active metabolite, O-desmethylvenlafaxine (ODV), are potent inhibitors of neuronal serotonin and norepinephrine reuptake and weak inhibitors of dopamine reuptake. That venlafaxine is analgesic is seen in studies in animals that show that venlafaxine is effective in reversing chronic neuropathic pain secondary to thermal hyperalgesia, and additionally is effective in treating the hyperalgesia of neuropathic pain due to chronic sciatic nerve constriction injury in rats (Lang E, Hord A H, Denson D. Venlafaxine hydrochloride (Effexor) relieves thermal hyperalgesia in rats with an experimental mononeuropathy. Pain 1998; 68:151-5). Venlafaxine-induced antinociception is significantly inhibited by naloxone, nor-BNI and naltrindole but not by β-FNA or naloxonazine, implying involvement of κ1- and δ-opioid mechanisms. When adrenergic and serotoninergic antagonists are used, yohimbine but not phentolamine or metergoline, decreased antinociception elicited by venlafaxine, implying a clear α2- and a minor α1-adrenergic mechanism of antinociception. Therefore, the antinociceptive effect of venlafaxine is mainly influenced by the κ- and δ-opioid receptor subtypes combined with the α2-adrenergic receptor. These results suggest a potential use of venlafaxine in the management of some pain syndromes. However, further research may be needed in order to establish both the exact clinical indications and the effective doses of venlafaxine when prescribed for neuropathic pain (Schreiber S, Backer M M, Pick C G. The antinociceptive effect of venlafaxine in mice is mediated through opioid and adrenergic mechanisms. Neuroscience Letters 1999; 273: 85-8).

Gabapentin (GBP; Neurontin; FIG. 2) is an anticonvulsant that has found increased utility for the treatment of clinical neuropathic pain. Although originally developed for the treatment of spasticity and epilepsy, recent attention has focused on the utility of GBP for the treatment of neuropathic pain based on its efficacy and minimal side-effect profile in clinical trials (Rice A S C and Maton S (2001) Gabapentin in postherpetic neuralgia: a randomised, double blind, placebo controlled study. Pain 94: 215-224). In rodent neuropathic pain models, GBP effectively attenuates thermal and mechanical hypersensitivity following peripheral nerve ligation (Hwang J H and Yaksh T L (1997) Effect of subarachnoid gabapentin on tactile-evoked allodynia in a surgically-induced neuropathic pain model in the rat. Reg Anesth 22: 249-256; Hunter J C, Gogas K R, Hedley L R, Jacobson L O, Kassotakis L, Thompson J, and Fontana D J (1997) The effect of novel anti-epileptic drugs in rat experimental models of acute and chronic pain. Eur J Pharmacol 324: 153-160). GBP has also been shown to inhibit thermal and mechanical hyperalgesia following carrageenan-induced inflammation (Lu Y and Westlund K N (1999) Gabapentin attenuates nociceptive behaviors in an acute arthritis model in rats. J Pharmacol Exp Ther 290: 214-219); however, other studies have reported limited effectiveness of GBP for inflammatory pain (Patel S, Naeem S, Kesingland A, Froestl W, Capogna M, Urban L, and Fox A (2001) The effects of GABAB agonists and gabapentin on mechanical hyperalgesia in models of neuropathic and inflammatory pain in the rat. Pain 90: 217-226). Additionally, GBP inhibits spontaneous nociceptive behaviors and mechanical hyperalgesia produced by intraplantar formalin or surgical incision, respectively (Field M J, Hughes J, and Singh L (2000) Further evidence for the role of the alpha2delta subunit of voltage dependent calcium channels in models of neuropathic pain. Br J Pharmacol 131: 282-286). The antinociceptive effects of GBP in models of neuropathic, inflammatory, and surgical pain appear to be selective for injury-induced hypersensitivity, since responses to acute noxious stimuli are unaffected (Field M J, Oles R J, Lewis A S, McCleary S, Hughes J, and Singh L (1997b) Gabapentin (neurontin) and S-(+)-3-isobutylgaba represent a novel class of selective antihyperalgesic agents. Br J Pharmacol 121: 1513-1522).

Despite growing interest in the analgesic properties of GBP, its mechanism of action remains unclear. Although it is a GABA analog, GBP does not bind GABA_{A} or GABA_{B} receptors or interact with GABA transporters (for review, see Taylor C P (2004) The biology and pharmacology of calcium channel alpha2-delta proteins. CNS Drug Rev 10: 183-188; Taylor C P, Gee N S, Su T Z, Kocsis J D, Welty D F, Brown J P, Dooley D J, Boden P, and Singh L (1998) A summary of mechanistic hypotheses of gabapentin pharmacology. Epilepsy Res 29: 233-249). GBP has been shown, however, to increase brain extracellular GABA levels in both rat and human studies (Loscher W, Honack D, and Taylor C P (1991) Gabapentin increases aminooxyacetic acid-induced GABA accumulation in several regions of rat brain. Neurosci Lett 128: 150-154). This increased extracellular GABA is likely due to either directly stimulated GABA release (Gu Y and Huang L Y M (2002) Gabapentin potentiates N-methyl-D-aspartate receptor mediated currents in rat GABAergic dorsal horn neurons. Neurosci Lett 324: 177-180) or changes in GABA metabolism via effects on glutamic acid decarboxylase and/or GABA-transaminase (Goldlust A, Su T, Welty D F, Taylor C P, and Oxender D L (1995) Effects of the anticonvulsant drug gabapentin on enzymes in the metabolic pathways of glutamate and GABA. Epilepsy Res 22: 1-11). The notion that GBP increases extracellular GABA is consistent with its effectiveness for neuropathic pain, since the pathology associated with this condition includes disruption of tonic inhibitory GABAergic transmission (Wiesenfeld-Hallin Z, Aldskogius H, Grant G, Hao J X, Hokfelt T, and Xu X J (1997) Central inhibitory dysfunctions: mechanisms and clinical implications. Behav Brain Sci 20: 420-425).

In addition to enhancing GABAergic transmission, it has been hypothesized that GBP modulates voltage-gated calcium channels, resulting in decreased neurotransmitter release. In support, GBP inhibits K⁺-evoked excitatory amino acid neurotransmitter release in neocortical and trigeminal nucleus slices (Fink K, Meder W, Dooley D J, and Gothert M (2000) Inhibition of neuronal Ca²⁺ influx by gabapentin and subsequent reduction of neurotransmitter release from rat neocortical slices. Br J Pharmacol 130: 900-906; Maneuf Y P, Hughes J, and McKnight A T (2001) Gabapentin inhibits the substance P-facilitated K⁺-evoked release of [³H]glutamate from rat caudial trigeminal nucleus slices. Pain 93: 191-196). Additionally, GBP has been shown to inhibit voltage gated calcium currents in dorsal root ganglia neurons (Sutton K G, Martin D J, Pinnock R D, Lee K, and Scott R H (2002) Gabapentin inhibits high-threshold calcium channel currents in cultured rat dorsal root ganglion neurones. Br J Pharmacol 135: 257-265). GBP-mediated inhibition of voltage-gated calcium channels would result in a reduction of excitatory transmission in the spinal cord dorsal horn, consistent with an inhibition of spinal nociceptive transmission (Shimoyama M, Shimoyama N, and Hori Y (2000) Gabapentin affects glutamatergic excitatory neurotransmission in the rat dorsal horn. Pain 85: 405-414).

Gabapentin and pregabalin are known to interact with both the α₂δ-1 and α₂δ-2 subunits (Klugbauer, N, Marais, E & Hofmann, F. (2003) J Bioenerg Biomembr 35, 639-647). The specific binding of gabapentin to α₂δ-1 was the first described interaction between a regulatory subunit of voltage activated calcium channels and a pharmaceutical agent. The discovery of the α₂δ subunit of voltage-gated calcium channels as a high-affinity binding site for GBP has further supported a role for voltage-gated calcium channels in its antinociceptive action (Brown J P, Dissanayake V U, Briggs A R, Milic M R and Gee N S (1998) Isolation of the [³H]gabapentin-binding protein α2δ Ca²⁺ channel subunit from porcine brain: development of a radioligand binding assay for α₂δ subunits using [³H]leucine. Anal Biochem 255: 236-243). Specific genes encoding three α₂δ subtypes have been identified (α₂δ-1, α₂δ-2, and α₂δ-3), with α₂δ-1 displaying the highest affinity for GBP (Marais E, Klugbauer N, and Hofmann F (2001) Calcium channel alpha2delta subunits-structure and gabapentin binding. Mol Pharmacol 59: 1243-1248). A specific role for α₂δ in neuropathic pain was originally described by Luo et al. (Luo Z D, Chaplan S R, Higuera E S, Sorkin L S, Stauderman K A, Williams M E, and Yaksh T L (2001) Upregulation of dorsal root ganglion alpha2delta calcium channel subunit and its correlation with allodynia in spinal nerve-injured rats. J Neurosci 21: 1868-1875), who found an increase in α₂δ expression in the dorsal root ganglion ipsilateral to the peripheral nerve injury that corresponded to the development of tactile allodynia. Additionally, Luo et al. (Luo Z D, Calcutt N A, Higuera E S, Valder C R, Song Y H, Svensson C I, and Myers R R (2002) Injury type-specific calcium channel alpha2delta-1 subunit up-regulation in rat neuropathic pain models correlates with antiallodynic effects of gabapentin. J Pharmacol Exp Ther 303: 1199-1205) reported that gabapentin efficacy was only evident in specific rat neuropathic pain models, in which increased α₂δ expression was observed. Further evidence supporting a role for α₂δ in the antinociceptive action of GBP was described in a recent study by Field et al. (Field, M J, Hughes, J & Singh, L. (2000) Br J Pharmacol 131, 282-286), in which the authors used two GBP analogs that stereoselectively interact with α₂δ: (1S,3R)₃-MeGBP (IC₅₀=42 nM) and (1R,3R)₃-MeGBP (IC₅₀>10,000 nM). The results demonstrated that whereas (1S,3R)₃-MeGBP effectively reversed neuropathy-induced allodynia, (1R,3R)₃-MeGBP was ineffective, supporting stereoselective efficacy related to α₂δ binding. Given these results, a series of experiments were designed to further compare the antinociceptive profiles of GBP and 3-MeGBP to better gauge the significance of the stereoselective efficacy of 3-MeGBP in terms of GBP action.

Urban et al. (Urban M O, Ren K, Park K T, Campbell B, Anker N, Stearns B, Aiyar J, Belley M, Cohen C, and Bristow L (2005) Comparison of the Antinociceptive Profiles of Gabapentin and 3-Methylgabapentin in Rat Models of Acute and Persistent Pain: Implications for Mechanism of Action. J Pharm Exp Therap 313: 1209-1216) have showed that GBP and 3-MeGBP have dissimilar antinociceptive profiles in models of acute and persistent pain, suggesting that their mechanisms of antinociceptive action differ.

It has been shown that α2δ-1 is up-regulated in DRG neurons after nerve injury (Luo, Z D, Calcutt, N A, Higuera, E S, Valder, C R, Song, Y H, Svensson, C I & Myers, R R J. (2002) Pharmacol Exp Ther 303, 1199-1205) and that this correlates with the onset and duration of pain behavior. Previous pharmacological investigations have provided evidence that analgesic activity of pregabalin and gabapentin is through the α2δ subunit. Thus, enantiomeric pairs of α2δ ligands show affinity-related potency in animal models of pain (Bryans 1999; Field 2000), and structure-activity relationships in models of epilepsy, anxiety, and pain, further demonstrate the importance of α₂δ binding to activity for pregabalin and its analogues (Belliotti 2005). Recent results are consistent with and add to these findings, demonstrating that it is the α₂δ-1 subunit that provides the requirement for the analgesic action of pregabalin and gabapentin (Field M J et al. (2006) Identification of the α2δ-1 subunit of voltage-dependent calcium channels as a molecular target for pain mediating the analgesic actions of pregabalin; PNAS; 103: 17537-17542).

Capsaicin is a natural constituent in pungent red chili peppers. Depending on the concentration used and the mode of application, capsaicin can selectively activate, desensitize, or exert a neurotoxic effect on small diameter sensory afferent nerves while leaving larger diameter afferents unaffected (Holzer P (1991). Capsaicin: cellular targets, mechanisms of action, and selectivity for thin sensory neurons. Pharmacol Rev 43: 143-201). Sensory neuron activation occurs due to interaction with a ligand-gated nonselective cation channel termed the vanilloid receptor (VR-1) (Caterina M J, Schumacher M A, Tominga M, Rosen T A, Levine J D and Julius D (1997). The capsaicin receptor: a heat-activated ion channel in the pain pathway. Nature (Lond) 389: 816-824), and receptor occupancy triggers Na⁺ and Ca²⁺ ion influx, action potential firing, and the consequent burning sensation associated with spicy food or capsaicin-induced pain. VR1 receptors are present on both C and Aδ fibers, and can be activated by capsaicin and its analogs, heat, acidification, and lipid metabolites (Caterina M J and Julius D (2001). The vanilloid receptor: a molecular gateway to the pain pathway. Annu Rev Neurosci 24: 487-517). Desensitization occurs with repeated administration of capsaicin, is a receptor-mediated process, and involves Ca²⁺- and calmodulin-dependent processes and phosphorylation of the cation channel (Winter J, Bevan S and Campbell E A (1995). Capsaicin and pain mechanisms. Br J Anaesth 75: 157-168; Wood J N and Docherty R (1997). Chemical activators of sensory neurons. Annu Rev Physiol 59: 457-482).

Capsaicin induces release of substance P and calcitonin gene-related peptide from both peripheral and central terminals of sensory neurons, and desensitization inhibits such release; such inhibition may result from inhibition of voltage-gated Ca²⁺-currents. Desensitization leads to analgesia in rodent paradigms, with specific characteristics of analgesia depending on the dose of capsaicin, route of administration, treatment paradigm (i.e., acute or repeated administration), and age of the animal.

Viral replication, immune regulation, and induction of various inflammatory and growth-regulatory genes require activation of a nuclear transcription factor (NF)-κ-B. Agents that can block NF-κ-B activation have potential to block downstream responses mediated through this transcription factor. Capsaicin (8-methyl-N-vanillyl-6-nonenamide) has been shown to regulate a wide variety of activities that require NF-κ-B activation (Singh S, Natarajan K, Aggarwal B B (1996). Capsaicin (8-methyl-N-vanillyl-6-nonenamide) is a potent inhibitor of nuclear transcription factor-KB activation by diverse agents. J. Immunol. 157:4412). The pretreatment of human myeloid ML-1a cells with capsaicin blocked TNF-mediated activation of NF-κ-B in a dose- and time-dependent manner. Capsaicin treatment of cells also blocked the degradation of I-κ-B alpha, and thus the nuclear translocation of the p65 subunit of NF-κ-B, which is essential for NF-κ-B activation. TNF-dependent promoter activity of I-κ-B alpha, which contains NF-κ⁻B binding sites, was also inhibited by capsaicin.

Acute intradermal injection of capsaicin to the skin in humans produces a burning sensation and flare response; the area of application becomes insensitive to mechanical and thermal stimulation, the area of flare exhibits a primary hyperalgesia to mechanical and thermal stimuli, and an area beyond the flare exhibits secondary allodynia (Simone D A, Baumann T K and LaMotte R H (1989). Dose-dependent pain and mechanical hyperalgesia in humans after intradermal injection of capsaicin. Pain 38: 99-107). Repeated application to normal skin produces desensitization to this response and thus forms the basis of the therapeutic use of topical capsaicin in humans. Desensitization involves both physiological changes in the terminals of the sensory neuron noted above, as well as a degree of loss of sensory fiber terminals within the epidermis (Nolano M, Simone D A, Wendelschafer-Crabb G, Johnson T, Hazen E and Kennedy W R (1999). Topical capsaicin in humans: parallel loss of epidermal nerve fibres and pain sensation. Pain 81: 135-145).

Topical capsaicin preparations of 0.025 and 0.075% are available for human use, and these produce analgesia in randomized double-blind placebo-controlled studies, open label trials, and clinical reports (Watson C P N. et al, (1993). A randomized vehicle-controlled trial of topical capsaicin in the treatment of post-herpetic neuralgia, Clin. Ther. 15:510-526). Topical capsaicin produces benefit in postherpetic neuralgia (Rains C, Bryson H M (1995). Topical capsaicin. A review of its pharmacological properties and therapeutic potential in post-herpetic neuralgia, diabetic neuropathy and osteoarthritis. Drugs Aging. 7(4):317-28), diabetic neuropathy (Capsaicin Study Group, 1992), postmastectomy pain syndrome (Dini D, Bertelli G, Gozza A and Formo G G (1993). Treatment of the post-mastectomy pain syndrome with topical capsaicin. Pain 54: 223-226), oral neuropathic pain, trigeminal neuralgia, and temperomandibular joint disorders (Epstein J B and Marcoe J H (1994). Topical capsaicin for treatment of oral neuropathic pain and trigeminal neuralgia. Oral Surg Oral Med Oral Pathol 77: 135-140; Hersh E V, Pertes R A and Ochs H A (1994). Topical capsaicin—pharmacology and potential role in the treatment of temperomandibular pain. J Clin Dent 5: 54-59), cluster headache (following intranasal application) (Marks D R, Rapoport A, Padla D, Weeks R, Rosum R, Sheftell F and Arrowsmith F (1993). A double-blind placebo-controlled trial of intranasal capsaicin for cluster headache. Cephalgia 13: 114-116), osteoarthritis (McCarthy G M and McCarthy D J (1992). Effect of topical capsaicin in the therapy of painful osteoarthritis of the hands. J Rheumatol 19: 604-607), and dermatological and cutaneous conditions (Hautkappe M, Roisen M F, Toledano A, Roth S, Jeffries J A and Ostermeier A M (1998). Review of the effectiveness of capsaicin for painful cutaneous disorders and neural dysfunction. Clin J Pain 14: 97-106). Whereas pain relief is widely observed in these studies, the degree of relief is usually modest, although some patients have a very good result. Topical capsaicin is generally not considered a satisfactory sole therapy for chronic pain conditions and is often considered an adjuvant to other approaches. No significant benefit was reported in chronic distal painful neuropathy (Low P A, Opfer-Gehrking T L, Dyck P J, Litchy W J and O'Brien P C (1995). Double-blind, placebo-controlled study of the application of capsaicin cream in chronic distal painful polyneuropathy. Pain 62: 163-168) or with human immunodeficiency virus-neuropathy (Paice J A, Ferrans C E, Lashley F R, Shott S, Vizgirda V and Pitrak D (2000). Topical capsaicin in the management of HIV-associated peripheral neuropathy. J Pain Symptom Manage 19: 45-52).

The distribution and metabolism of capsaicin and/or dihydrocapsaicin has been studied in rats. Capsaicin is distributed to the brain, spinal cord, liver and blood within 20 minutes of i.v. administration. Oral doses of dihydrocapsaicin in the rat showed metabolic activity associated with its absorption into the portal vein. Capsaicin and dihydrocapsaicin are metabolized in the liver by the mixed-function oxidation system (cytochrome P-450-dependent system). It is assumed that capsaicin is excreted in urine. In rats, most of dihydrocapsaicin is known to be rapidly metabolized and excreted in the urine (Rumsfield, J A, and West D (1991). Topical capsaicin in dermatological and peripheral pain disorders. DICP, Ann. Pharmacotherap. 25: 381-387).

Oral dosing of rats with capsaicin and dihydrocapsaicin results in an 85% absorption in the jejunum after 3 hours. With respect to topical applications of capsaicin, it has been estimated that assuming 100% of a topically-applied dose is absorbed into the body, an application of 90 g capsaicin (2 tubes of cream, 0.025% capsaicin) per week would result in a daily exposure of 0.064 mg/kg capsaicin for a 50 kg person. This represents less than 10% of the dietary intake of a typical Indian or Thai diet (Rumsfield, J A, and West D (1991). Topical capsaicin in dermatological and peripheral pain disorders. DICP, Ann. Pharmacotherap. 25: 381-387).

The most frequently encountered adverse effect with capsaicin is burning pain at the site of application, particularly in the first week of application. This can make it impossible to blind trials and can lead to dropout rates ranging from 33 to 67% (Paice J A, Ferrans C E, Lashley F R, Shott S, Vizgirda V and Pitrak D (2000). Topical capsaicin in the management of HIV-associated peripheral neuropathy. J Pain Symptom Manage 19: 45-52). Another factor in compliance is the time delay before therapeutic effect is observed (at least a week, but sometimes several weeks). One approach toward minimizing adverse effects and accelerating the rate of analgesia has been to deliver a higher capsaicin concentration (5-10%) under regional anesthesia, and this produced sustained analgesia lasting 1 to 8 weeks in cases of complex regional pain syndrome and neuropathic pain (Robbins W R, Staats P S, Levine J, et al. Treatment of intractable pain with topical large-dose capsaicin: preliminary report. Anesth Analg 1998; 86:579-83). When topical local anesthetics were applied with 1% topical capsaicin, no alteration in pain produced by the capsaicin was observed in healthy subjects (Fuchs P N, Pappagallo M and Meyer R A (1999). Topical EMLA^{R} pre-treatment fails to decrease the pain induced by 1% topical capsaicin. Pain 80: 637-642) indicating that this cotreatment was not sufficient to block the pain induced by capsaicin.

Magnesium participates in numerous enzymatic reactions including all reactions that involve the formation and utilization of adenosine-50-triphosphate (ATP) in energy metabolism. Whenever neurons cannot generate sufficient ATP to keep their ion pumps working properly, membranes depolarize and excessive Ca2+ leaks into cells, triggering the synaptic release of glutamate, which further depolarizes neurons, further raising intracellular Ca2+ which causes even more glutamate to be released repeating in endless cycles [Iseri L T, French J H. Magnesium: nature's physiologic calcium blocker. Am Heart J 1984; 108:188-93] resulting in neuronal dysfunction and depression. Magnesium has been shown to cause a dose-dependent inhibition of platelet aggregation (Ravn H. B., Korsholm T. L., Falk E. Oral magnesium supplementation induces favorable antiatherogenic changes in ApoE-deficient mice. Arterioscler Thromb Vasc Biol 2001; 21:858-862.). Magnesium has a strong vascular dilating effect, lending support to the vascular theory of migraine. IMg2+ levels are known to affect entry of Ca2+, and intracellular ICa2+ from sarcoplasmic and endoplasmic reticulum, in vascular smooth muscle and vascular endothelial cells and to control vascular tone and reactivity to endogenous hormones and neurotransmitters. Cerebral blood vessel muscle cells are particularly sensitive to IMg2+; Mg deficiency results in contraction and potentiation of vasoconstrictors and excess IMg2+ results in vasodilatation and inhibition of vasoconstrictors (Yang, Z. W., Gebrewold, A., Nowakowski, M., Altura, B. T., and Altura, B. M. (2000). Mg²⁺-induced endothelium-dependent relaxation of blood vessels and blood pressure lowering: role of NO. Am. J. Physiol. Regulatory Integrative Comp. Physiol. 278, R628-R639; Yang, Z. W., Wang, J., Zheng, T., Altura, B. T., and Altura, B. M. (2000). Low [Mg²⁺]o, induces contraction of cerebral arteries: roles of tyrosine and mitogen-activated protein kinases. Am. J. Physiol. Heart Circ. Physiol. 279, H185-H194).

Magnesium is intimately involved in the control of N-methyl-D-aspartate (NMDA) glutamate receptors which play an important role in pain transmission in the nervous system (Foster A C, Fagg G E. Neurobiology. Taking apart NMDA Receptors. Nature 1987; 329:395-396) and regulation of cerebral blood flow (Huang Q. F., Gebrewold A., Zhang A, Altura B. T., Altura B. M. Role of excitatory amino acids in regulation of rat pial microvasculature. Am J Physiol 1994; 266:R158—R163). Magnesium ion plugs the NMDA receptor and prevents calcium ions from entering the cell. Lowering Mg2+ concentration facilitates activation of the NMDA receptor, which allows calcium to enter the cell and exert its effects both on neurons and cerebral vascular muscle. Thus magnesium can be considered an NMDA receptor antagonist at several important sites.

Magnesium is involved in many central nervous processes both at presynaptic and postsynaptic levels. Changes in magnesium concentration exert diverse influences on neurons, in normal or pathological conditions.

Tricyclic antidepressants (FIG. 4) are considered first-line systemic therapy for many neuropathic pain syndromes, including diabetic neuropathy (Galer B S. Neuropathic pain of peripheral origin: advances in pharmacologic treatment. Neurology 1995; 45(suppl 9):S17-25). In a literature review, 6 of 13 controlled trials evaluating antidepressants for diabetic neuropathy showed clinically significant pain relief with use of tricyclic antidepressants (McQuay H J, Tramèr M, Nye B A, Carroll D, Wiffen P J, Moore R A. A systematic review of antidepressants in neuropathic pain. Pain 1996; 68:217-27; Sindrup S H, Jensen T S. Efficacy of pharmacological treatments of neuropathic pain: an update and effect related to mechanism of drug action. Pain 1999; 83:389-400). Electrotherapy may enhance the response of patients with a partial response to tricyclic antidepressants in diabetic neuropathy (Kumar D, Alvaro M S, Julka I S, et al. Diabetic peripheral neuropathy: effectiveness of electrotherapy and amitriptyline for symptomatic relief. Diabetes Care 1998; 21:1322-5). However, no differences in the effectiveness of different tricyclic antidepressants were shown (Lipman A G. Analgesic drugs for neuropathic and sympathetically maintained pain. Clin Geriatr Med 1996; 12:501-15). Thus, the appropriate choice of tricyclic antidepressant depends on each agent's adverse-effect profile. Current literature supports the use of tricyclic antidepressants for the management of postherpetic neuralgia (Johnson R W. Herpes zoster and postherpetic neuralgia: optimal treatment. Drugs Aging 1997; 10:80-94), and up to 67% of patients respond to tricyclic antidepressant treatment (Bowsher D. Post-herpetic neuralgia in older patients: incidence and optimal treatment. Drugs Aging 1994; 5: 411-18). Central poststroke pain is pain that occurs in body areas that have lost part of their sensory innervation due to a stroke. Tricyclic anti-depressants have been considered first-line systemic therapy for central poststroke pain (Bryson H M, Wilde M I. Amitriptyline: a review of its pharmacological properties and therapeutic use in chronic pain states. Drugs Aging 1996; 8:459-76), although their use for central poststroke pain relief is supported only by isolated case reports (Bryson 1996) and one placebo-controlled trial in which amitriptyline demonstrated superior relief compared with that of placebo in 10 (67%) of 15 patients (Leijon G, Boivie J K. Central post-stroke pain: a controlled trial of amitriptyline and carbamazepine. Pain 1989; 36:27-36). Some evidence exists that early onset of antidepressant therapy (<1 yr after stroke) can positively affect treatment success in central poststroke pain (Bowsher D. The management of central post-stroke pain. Postgrad Med J 1995; 71:598-604).

Tricyclic antidepressants may be useful as adjunctive therapy for cancer-related neuropathic pain syndromes (Jacox A, Carr D B, Payne R, et al. Clinical practice guideline number 9: management of cancer pain. Rockville, Md.: U.S. Department of Health and Human Services, Agency for Health Care Policy and Research, 1994. AHCPR publication no. 94-0592). Tricyclic antidepressants provide pain relief by independently providing analgesia specific for neuropathic pain, potentiating the effect of opioids, and improving underlying depression and insomnia (Levy M H. Pharmacologic treatment of cancer pain. N Engl J Med 1996; 335:1124-32). Although controlled trials using tricyclic anti-depressants in patients with cancer are limited and most data about tricyclic antidepressant analgesic effectiveness have been obtained with other chronic pain syndromes, these agents are accepted as adjunctive analgesics for cancer pain.

Tricyclic antidepressants should be administered cautiously in patients with angle-closure glaucoma, benign prostatic hypertrophy, urinary retention, constipation, cardiovascular disease, or impaired liver function. The agents should be avoided in patients with second- or third-degree heart block, arrhythmias, prolonged QT interval on the electrocardiogram, or severe liver disease and in patients who have had a recent acute myocardial infarction.

The adverse effects of tricyclic antidepressants are well known, but their prevalence rates vary by agent and patient group. In general, elderly patients experience a higher frequency of adverse effects, and slow dosage titration is recommended (Rudorfer M V, Manji H K, Potter W Z. Comparative tolerability profiles of the newer versus older antidepressants. Drug Saf 1994; 10:18-46). The most common adverse effects of tricyclic antidepressants (constipation, dry mouth, blurred vision, cognitive changes, tachycardia, urinary hesitation) are associated with their anticholinergic activity. Other common adverse effects are orthostatic hypotension, falls, weight gain, and sedation. In general, the secondary amines (e.g., desipramine, nortriptyline) exhibit fewer anticholinergic and sedative effects than do the tertiary amines (e.g., amitriptyline, imipramine, doxepin); therefore, the secondary amines may be more desirable in the elderly population (Lipman A G. Analgesic drugs for neuropathic and sympathetically maintained pain. Clin Geriatr Med 1996; 12:501-15).

Neuropathic pain generally responds more quickly than depression to tricyclic antidepressants (i.e., 3-10 days vs 2-4 wks) and often with one-third to one-half the dosage administered for depression (Billings J A. Neuropathic pain. J Palliat Care 1994; 10:40-3). Owing to their improved adverse-effect profiles, therapy with one of the secondary amine tricyclic antidepressants should be considered in elderly patients. For desipramine, nortriptyline, amitriptyline, and imipramine, a starting dosage of 10 mg at bedtime is recommended, with dosage increments of 10-25 mg made no more frequently than every 5-7 days (Galer B S. Painful polyneuropathy: diagnosis, pathophysiology, and management. Semin Neurol 1994; 14:237-46). Although the timing of administration will not affect a tricyclic antidepressant's analgesic activity, bedtime administration is recommended to take advantage of the sedative activity. The tricyclic antidepressant dosage should depend on the degree of pain relief and emergence of adverse effects. A daily dose of 50-100 mg of the above agents is usually effective (Emanuele N V, Emanuele M A. Drugs to treat the tissue complications of diabetes: peripheral neuropathy. Compr Ther 1995; 21:579-82). If a patient reaches a dosage of 75-100 mg/day without sufficient pain relief, determination of the serum drug concentration may be reasonable to evaluate whether presumably adequate drug concentrations have been achieved. Remember, no accepted therapeutic drug concentration range exists for analgesia.

Not all patients respond to tricyclic anti-depressant therapy within 10 days of initiation or with lower dosages. Some patients may require higher dosages and several weeks of treatment before efficacy is evident. Patients are often referred to specialty pain clinics because the tricyclic antidepressant dosage was not adequate. In addition, these drugs may be discontinued unnecessarily because of adverse effects caused by starting them at inappropriately high dosages, titrating the dosage upward too rapidly, or starting several drugs at one time. An adequate trial must be given before failure of a tricyclic antidepressant is determined. Failure of one tricyclic antidepressant does not preclude success with a different agent, and the practitioner should consider trying two or, perhaps, three agents sequentially before substituting another therapeutic option.

Patients abruptly withdrawn from a tricyclic antidepressant may experience withdrawal that manifests as any of a variety of clinical symptoms (e.g., malaise, insomnia, drowsiness, anorexia, muscle aches, apathy, headache, mania, profuse sweating, irritability, abdominal pains, diarrhea, nausea, vivid and terrifying dreams, movement disorders). To avoid a withdrawal syndrome, a slow taper over 2-4 weeks (depending on the dosage) is recommended (Garner 1993).

Amitriptyline is a tricyclic agent used for the treatment of major depression (Baldessarini R J (1995) Drugs and the treatment of psychiatric disorders, in The Pharmacological Basis of Therapeutics (Hardman J G, Limbird L E, Molinoff P B, Ruddon R W and Gilman A G eds) pp 431-459, McGraw-Hill, New York). Amitriptyline, nortriptyline, and desipramine have been established as analgesics independent of their antidepressant effects. Although their mechanism of analgesic action has not been clearly defined, tricyclic antidepressants are thought to have an inhibitory effect on nociceptive pathways by blocking the reuptake of serotonin and norepinephrine (Calissi P T, Jaber L A. Peripheral diabetic neuropathy: current concepts in treatment. Ann Pharmacother 1995; 29:769-77). Originally, the major mechanism of the analgesic effect of tricyclic antidepressants was believed to be related to serotonin reuptake inhibition. However, the selective serotonin reuptake inhibitor antidepressants have not demonstrated substantial effectiveness in neuropathic pain. Animal models of peripheral neuropathic pain have shown that tricyclic antidepressants act as sodium channel blockers, similar to local anesthetic and antiarrhythmic agents.

Amitriptyline drug is effective in the treatment of postherpetic neuralgia, diabetic neuropathy, and other neuropathic pain syndromes. Oral amitriptyline achieves a good or moderate response in about two-thirds of patients with postherpetic neuralgia and three-quarters of patients with painful diabetic neuropathy; such neurogenic pain syndromes are often unresponsive to narcotic analgesics (Bryson H M and Wilde M I (1996) Amitriptyline. A review of its pharmacological properties and therapeutic use in chronic pain states. Drugs Aging 8: 459-476). Whether analgesic effects of amitriptyline are linked to its mood-altering activity and/or are attributable to a discrete pharmacological action is unknown. Above the therapeutic plasma concentration of 0.3 to 0.8 μM, the tricyclic antidepressants have significant effects on the cardiovascular system, including direct depression of the myocardium and evidence of prolonged conduction times (Nattel S (1985) Frequency-dependent effects of amitriptyline on ventricular conduction and cardiac rhythm in dogs. Circulation 72: 898-906); with an overdose of >3 μM, these effects may be life-threatening (Amsterdam J, Brunswick D and Mendels J (1980) The clinical application of tricyclic antidepressant pharmacokinetics and plasma levels. Am J Psychiatry 137: 653-662). The known physiological targets of tricyclic antidepressants in the central nervous system are the 5-HT₂ serotonin receptors and the α₁-adrenergic receptors.

In addition to these primary targets, tricyclic antidepressants are also effective K⁺ and Na⁺ channel blockers. For example, tricyclic imipramine inhibits transient K⁺ channels in hippocampal neurons with an IC₅₀ of ˜6 μM (Kuo, 1998). In adrenal chromaffin cells, amitriptyline blocks peak Na⁺ currents with an IC₅₀ value of 20.2 μM (Pancrazio J J, Kamatchi G L, Roscoe A K and Lynch C (1998) Inhibition of neuronal Na⁺ channels by antidepressant drugs. J Pharmacol Exp Ther 284: 208-214).

Another anti-depressant milnacipran and methods for its synthesis are described in U.S. Pat. No. 4,478,836. Milnacipran (midalcipran, midacipran, F2207) inhibits the uptake of both, norepinephrine (NE) and serotonin (5-HT), with an NE to 5-HT ratio of 2:1 (Moret C, Charveron M, Finberg J P, Couzinier J P, Briley M (1985). “Biochemical profile of midalcipran (F 2207), 1-phenyl-1-diethyl-aminocarbonyl-2-aminomethyl-cyclopropane (Z) hydrochloride, a potential fourth generation antidepressant drug”. Neuropharmacology 24 (12): 1211-9) but does not affect the uptake of dopamine. Milnacipran has no affinity for a or β adrenergic, muscarinic, histaminergic, and dopaminergic receptors. This suggests that milnacipran has a low potential to produce anticholinergic, sedative, and stimulant effects. Milnacipran does not affect the number of beta adrenoceptors in rat cortex after chronic administration (Briley M, Prost J F, Moret C (1996). “Preclinical pharmacology of milnacipran”. International clinical psychopharmacology 11 Suppl 4: 9-14). Additional information regarding milnacipran may be found in the Merck Index, 12th Edition, at entry 6281.

Milnacipran (Ixel©, Pierre Fabre), has demonstrated numerous adverse reactions in human clinical trials with tolerability decreasing with increasing dose (Puech A. et al., 1997, Int. Clin. Psychopharm., 12:99-108). In the double-blind, randomized, multicenter clinical study the most frequent spontaneously reported adverse events for 100 mg/day milnacipran twice daily were as follows: abdominal pain (13%), constipation (10%), and headache (9%). Interestingly, when in the same study milnacipran was given 200 mg/day twice daily, pain related adverse reactions decreased (headache to 8% and abdominal pain to 7%) but nausea and vomiting were more pronounced side effects and were reported by 7% of the patients (Guelfi J. D., 1998, Int. Clin. Psychopharm., 13:121-128). In a double-blind comparative study involving 219 elderly patients with depression the only adverse event reported more frequently for milnacipran recipients than for TCA imipramine recipients was nausea. Patients received either milnacipran or imipramine 75 100 mg/day twice daily for 8 weeks. It was also observed that when milnacipran was administered intravenously to 10 patients, five of them reported transient nausea. Nausea was primarily reported at the moment of peak of milnacipran plasma level. This study clearly demonstrates that nausea is directly correlated with the milnacipran blood plasma concentration. In addition, it strongly suggests that the nausea can be a centrally mediated side effect since the drug was given intravenously in this study. Data from other studies suggest that milnacipran may also induce a locally mediated nausea via gastric irritation (the rapid onset of the nausea was observed even prior to achieving peak plasma levels). The incidence of certain adverse events increases with dosage, including nausea, vomiting, sweating, hot flashes, palpitations, tremor, anxiety, dysuria, and insomnia.

It is important to note that in one of the early depression trials, even after one week of milnacipran dose escalation employed to reduce side effects, the most commonly reported reason for discontinuation of treatment because of adverse effects was nausea and vomiting (Leinonen E, Lepola U, Koponen H, Mehtonen O P, Rimon R (1997). “Long-term efficacy and safety of milnacipran compared to clomipramine in patients with major depression”. Acta psychiatrica Scandinavica 96 (6): 497-504). In the recent fibromyalgia clinical trial with the long dose escalation period (four weeks) which was implemented in order to reduce milnacipran side effects and increase patient's tolerance, the most common dose-related side effect reported by patients was nausea (Cypress Bioscience Inc., Cypress Bioscience Inc. Announces Final Results of Milnacipran Phase II Clinical Trial in Fibromyalgia, Media Release, Mar. 21, 2003).

The currently immediate available release formulation of milnacipran is not suitable for the treatment of health conditions that require milnacipran doses equal or above 100 mg/day given either as once a day or twice a day due to high incidence of treatment-emergent side effects that leads to poor patient's tolerance. Higher doses are required in the treatment of severe depression and other associated disorders. As shown in one of the early antidepressant clinical trials, milnacipran dosage of 200 mg/day was superior to the lower doses (Von Frenckell R et al., 1990, Int. Clin. Psychopharmacology, 5:49-56). Milnacipran dosing regime of 100-250 mg daily was recently reported for the treatment of fibromyalgia (U.S. Pat. No. 6,602,911). It would be very difficult to reach the upper limits of the dose range using the currently available formulation due to the dose related treatment emergent side effects and the need to titrate over a long period to reach the required dose.

Dextromethorphan is typically administered orally. As an antitussive, the recommended dosage for adults is 60-120 mg daily in divided doses. Each current FDA approved brand contains different quantities of dextromethorphan, generally 20-30 mg per dose. Approximate doses are: threshold dose 80-90 mg; light 100-200 mg; common 200-400 mg; strong 400-600 mg; and heavy dose 600-1500 mg.

At recommended doses, dextromethorphan produces little or no CNS depression. At higher doses, positive effects may include acute euphoria, elevated mood, dissociation of mind from body, creative dream-like experiences, and increased perceptual awareness. Other effects include disorientation, confusion, pupillary dilation, and altered time perception, visual and auditory hallucinations, and decreased sexual functioning. Doses of approximately 100-200 mg have a mild, stimulant effect (likened to MDA); doses of 200-500 mg produce a more intoxicating effect (likened to being ‘drunk and stoned’); 500-1000 mg may result in mild hallucinations and a mild dissociate effect (likened to a low dose of ketamine) and an overall disturbance in thinking, senses and memory; while doses over 1000 mg may produce a fully dissociative effect (likened to a high dose of ketamine). Abused doses are capable of impairing judgment, memory, language, and other mental performances.

Tramadol has been given is single oral doses of 50, 75, and 100 mg to patients with pain following surgical procedures and pain following oral surgery. In single-dose models of pain following oral surgery, pain relief was demonstrated in some patients at doses of 50 and 75 mg. A dose of 100 mg tended to provide analgesia superior to codeine sulfate 60 mg, but it was not as effective as the combination of aspirin 650 mg with codeine phosphate 60 mg.

Tramadol has been studied in three long-term controlled trials involving a total of 820 patients, with 530 patients receiving tramadol. Patients with a variety of chronic painful conditions were studied in double-blind trials of one to three months duration. The average daily doses of approximately 250 mg tramadol in divided doses were generally comparable to five doses of acetaminophen 300 mg with codeine phosphate 30 mg (T#3) daily, five doses of aspirin 325 mg with codeine phosphate 30 mg daily, or two to three doses of acetaminophen 500 mg with oxycodone hydrochloride 5 mg daily. Tramadol 50 to 100 mg can be administered in adults over 17 years of age as needed for pain relief every 4 to 6 hours not to exceed 400 mg per day.

The recommended daily dose of tramadol for treating neuropathic pain is between 50 and 100 mg every 4 to 6 hours, with a maximum dose of 400 mg/day. The duration of the analgesic effect after a single oral dose of tramadol 100 mg is about 6 hours. Adverse effects, and nausea in particular, are dose dependent and therefore considerably more likely to appear if the loading dose is high. The reduction of this dose during the first days of treatment is an important factor in improving tolerability. Other adverse effects are generally similar to those of opiates, although they are usually less severe, and can include respiratory depression, dysphoria and constipation.

Gabapentin is commercially supplied as Neurontin® Capsules, Neurontin Tablets, and Neurontin Oral Solution, as imprinted hard shell capsules containing 100 mg, 300 mg, and 400 mg of gabapentin, elliptical film-coated tablets containing 600 mg and 800 mg of gabapentin or an oral solution containing 250 mg/5 mL of gabapentin. Gabapentin bioavailability is not dose proportional; i.e., as dose is increased, bioavailability decreases. Bioavailability of gabapentin is approximately 60%, 47%, 34%, 33%, and 27% following 900, 1200, 2400, 3600, and 4800 mg/day given in 3 divided doses, respectively. Food has only a slight effect on the rate and extent of absorption of gabapentin (14% increase in AUC and Cₘₐₓ). Less than 3% of gabapentin circulates bound to plasma protein. The apparent volume of distribution of gabapentin after 150 mg intravenous administration is 58±6 L (Mean±SD). In patients with epilepsy, steady-state predose (Cₘᵢₙ) concentrations of gabapentin in cerebrospinal fluid were approximately 20% of the corresponding plasma concentrations. Gabapentin is eliminated from the systemic circulation by renal excretion as unchanged drug. Gabapentin is not appreciably metabolized in humans. Gabapentin elimination half-life is 5 to 7 hours and is unaltered by dose or following multiple dosing. Gabapentin elimination rate constant, plasma clearance, and renal clearance are directly proportional to creatinine clearance. In elderly patients, and in patients with impaired renal function, gabapentin plasma clearance is reduced. Gabapentin can be removed from plasma by hemodialysis.

Currently gabapentin is indicated as adjunctive therapy in the treatment of partial seizures with and without secondary generalization in patients over 12 years of age with epilepsy. Gabapentin is also indicated as adjunctive therapy in the treatment of partial seizures in pediatric patients age 3¾ 12 years.

Gabapentin is not appreciably metabolized nor does it interfere with the metabolism of commonly coadministered antiepileptic drugs. Gabapentin is given orally with or without food. In adults with postherpetic neuralgia, Gabapentin therapy may be initiated as a single 300-mg dose on Day 1, 600 mg/day on Day 2 (divided BID), and 900 mg/day on Day 3 (divided TID). The dose can subsequently be titrated up as needed for pain relief to a daily dose of 1800 mg (divided TID). In clinical studies, efficacy was demonstrated over a range of doses from 1800 mg/day to 3600 mg/day with comparable effects across the dose range. Additional benefit of using doses greater than 1800 mg/day was not demonstrated. For patients >12 years of age: The effective dose of gabapentin is 900 to 1800 mg/day and given in divided doses (three times a day) using 300 or 400 mg capsules, or 600 or 800 mg tablets. The starting dose is 300 mg three times a day. If necessary, the dose may be increased using 300 or 400 mg capsules, or 600 or 800 mg tablets three times a day up to 1800 mg/day. Dosages up to 2400 mg/day have been well tolerated in long-term clinical studies. Doses of 3600 mg/day have also been administered to a small number of patients for a relatively short duration, and have been well tolerated. The maximum time between doses in the TID schedule should not exceed 12 hours.

The most commonly observed adverse events associated with the use of gabapentin in adults, not seen at an equivalent frequency among placebo-treated patients, were dizziness, somnolence, and peripheral edema. The most commonly observed adverse events associated with the use of gabapentin in combination with other antiepileptic drugs in patients >12 years of age, not seen at an equivalent frequency among placebo-treated patients, were somnolence, dizziness, ataxia, fatigue, and nystagmus. The most commonly observed adverse events reported with the use of gabapentin in combination with other antiepileptic drugs in pediatric patients 3 to 12 years of age, not seen at an equal frequency among placebo-treated patients, were viral infection, fever, nausea and/or vomiting, somnolence, and hostility.

In adults with postherpetic neuralgia, gabapentin therapy may be initiated as a single 300-mg dose on Day 1, 600 mg/day on Day 2 (divided BID), and 900 mg/day on Day 3 (divided TID). The dose can subsequently be titrated up as needed for pain relief to a daily dose of 1800 mg (divided TID). In clinical studies, efficacy was demonstrated over a range of doses from 1800 mg/day to 3600 mg/day with comparable effects across the dose range. Additional benefit of using doses greater than 1800 mg/day was not demonstrated. For patients >12 years of age: The effective dose of Neurontin is 900 to 1800 mg/day and given in divided doses (three times a day) using 300 or 400 mg capsules, or 600 or 800 mg tablets. The starting dose is 300 mg three times a day. If necessary, the dose may be increased using 300 or 400 mg capsules, or 600 or 800 mg tablets three times a day up to 1800 mg/day. Dosages up to 2400 mg/day have been well tolerated in long-term clinical studies. Doses of 3600 mg/day have also been administered to a small number of patients for a relatively short duration, and have been well tolerated. The maximum time between doses in the TID schedule should not exceed 12 hours.

The most commonly observed adverse events associated with the use of gabapentin in adults, not seen at an equivalent frequency among placebo-treated patients, were dizziness, somnolence, and peripheral edema. The most commonly observed adverse events associated with the use of gabapentin in combination with other antiepileptic drugs in patients >12 years of age, not seen at an equivalent frequency among placebo-treated patients, were somnolence, dizziness, ataxia, fatigue, and nystagmus. The most commonly observed adverse events reported with the use of gabapentin in combination with other antiepileptic drugs in pediatric patients 3 to 12 years of age, not seen at an equal frequency among placebo-treated patients, were viral infection, fever, nausea and/or vomiting, somnolence, and hostility.

Pregabalin, an analog of gabapentin, is sold commercially as LYRICA capsules and is administered orally and are supplied as imprinted hard-shell capsules containing 25, 50, 75, 100, 150, 200, 225, and 300 mg of pregabalin, along with lactose monohydrate, cornstarch, and talc as inactive ingredients. The capsule shells contain gelatin and titanium dioxide. In addition, the orange capsule shells contain red iron oxide and the white capsule shells contain sodium lauryl sulfate and colloidal silicon dioxide. Colloidal silicon dioxide is a manufacturing aid that may or may not be present in the capsule shells. The imprinting ink contains shellac, black iron oxide, propylene glycol, and potassium hydroxide.

Treatment with pregabalin 100 and 200 mg three times a day statistically significantly improved the endpoint mean pain score and increased the proportion of patients with at least a 50% reduction in pain score from baseline. There was no evidence of a greater effect on pain scores of the 200 mg three times a day dose than the 100 mg three times a day dose, but there was evidence of dose dependent adverse reactions. A 13-week study compared pregabalin 75, 150, and 300 mg twice daily with placebo. Patients with creatinine clearance (CLcr) between 30 to 60 mL/min were randomized to 75 mg, 150 mg, or placebo twice daily. Patients with creatinine clearance greater than 60 mL/min were randomized to 75 mg, 150 mg, 300 mg or placebo twice daily. In patients with creatinine clearance greater than 60 mL/min treatment with all doses of pregabalin statistically significantly improved the endpoint mean pain score and increased the proportion of patients with at least a 50% reduction in pain score from baseline. Despite differences in dosing based on renal function, patients with creatinine clearance between 30 to 60 mL/min tolerated pregabalin less well than patients with creatinine clearance greater than 60 mL/min as evidenced by higher rates of discontinuation due to adverse reactions.

An 8-week study compared pregabalin 100 or 200 mg three times a day with placebo, with doses assigned based on creatinine clearance. Patients with creatinine clearance between 30 to 60 mL/min were treated with 100 mg three times a day, and patients with creatinine clearance greater than 60 mL/min were treated with 200 mg three times daily. Treatment with pregabalin statistically significantly improved the endpoint mean pain score and increased the proportion of patients with at least a 50% reduction in pain score from baseline.

A 8-week study compared pregabalin 50 or 100 mg three times a day with placebo with doses assigned regardless of creatinine clearance. Treatment with pregabalin 50 and 100 mg three times a day statistically significantly improved the endpoint mean pain score and increased the proportion of patients with at least a 50% reduction in pain score from baseline. Patients with creatinine clearance between 30 to 60 mL/min tolerated pregabalin less well than patients with creatinine clearance greater than 60 mL/min as evidenced by markedly higher rates of discontinuation due to adverse reactions.

A 14-week study compared pregabalin total daily doses of 300 mg, 450 mg and 600 mg with placebo. Patients were enrolled with a minimum mean baseline pain score of greater than or equal to 4 on an 11-point numeric pain rating scale and a score of greater than or equal to 40 mm on the 100 mm pain visual analog scale (VAS). The baseline mean pain score in this trial was 6.7. Responders to placebo in an initial one-week run-in phase were not randomized into subsequent phases of the study. A total of 64% of patients randomized to pregabalin completed the study. There was no evidence of a greater effect on pain scores of the 600 mg daily dose than the 450 mg daily dose, but there was evidence of dose-dependent adverse reactions.

The maximum recommended dose of pregabalin for neuropathic pain associated with diabetic peripheral neuropathy is 100 mg three times a day (300 mg/day) in patients with creatinine clearance of at least 60 mL/min. Dosing should begin at 50 mg three times a day (150 mg/day) and may be increased to 300 mg/day within 1 week based on efficacy and tolerability. Because pregabalin is eliminated primarily by renal excretion, the dose should be adjusted for patients with reduced renal function. Although pregabalin was also studied at 600 mg/day, there is no evidence that this dose confers additional significant benefit and this dose was less well tolerated. In view of the dose-dependent adverse reactions, treatment with doses above 300 mg/day is not recommended.

The recommended dose of pregabalin for fibromyalgia is 300 to 450 mg/day. Dosing should begin at 75 mg two times a day (150 mg/day) and may be increased to 150 mg two times a day (300 mg/day) within 1 week based on efficacy and tolerability. Patients who do not experience sufficient benefit with 300 mg/day may be further increased to 225 mg two times a day (450 mg/day). Although pregabalin was also studied at 600 mg/day, there is no evidence that this dose confers additional benefit and this dose was less well tolerated. In view of the dose-dependent adverse reactions, treatment with doses above 450 mg/day is not recommended. Because pregabalin is eliminated primarily by renal excretion, the dose should be adjusted for patients with reduced renal function (creatinine clearance less than 60 mL/min—see Patients with Renal Impairment).

In clinical trials in patients with neuropathic pain associated with diabetic peripheral neuropathy, 9% of patients treated with pregabalin and 4% of patients treated with placebo discontinued prematurely due to adverse reactions. In the pregabalin treatment group, the most common reasons for discontinuation due to adverse reactions were dizziness (3%) and somnolence (2%). In comparison, <1% of placebo patients withdrew due to dizziness and somnolence. Other reasons for discontinuation from the trials, occurring with greater frequency in the pregabalin group than in the placebo group, were asthenia, confusion, and peripheral edema. Each of these events led to withdrawal in approximately 1% of patients.

In clinical trials of patients with fibromyalgia, 19% of patients treated with pregabalin (150-600 mg/day) and 10% of patients treated with placebo discontinued prematurely due to adverse reactions. In the pregabalin treatment group, the most common reasons for discontinuation due to adverse reactions were dizziness (6%) and somnolence (3%). In comparison, <1% of placebo-treated patients withdrew due to dizziness and somnolence. Other reasons for discontinuation from the trials, occurring with greater frequency in the pregabalin treatment group than in the placebo treatment group, were fatigue, headache, balance disorder, and weight increased. Each of these adverse reactions led to withdrawal in approximately 1% of patients.

Various capsaicin compositions have been developed over the years, in particular, the psoriatic composition of U.S. Pat. No. 4,486,450, the nasal composition of U.S. Pat. No. 5,134,166, and the composition of U.S. Pat. No. 4,997,853, the anti-inflammatory composition of U.S. Pat. No. 5,560,910, the composition of U.S. Pat. No. 5,962,532, the composition for animals of U.S. Pat. No. 5,916,565, the stomach treatments of U.S. Pat. No. 5,889,041, the composition of U.S. Pat. No. 5,827,886, the patch with medication of U.S. Pat. No. 5,741,510, all of which are incorporated by reference herein.

U.S. Pat. No. 6,593,370 discloses a topical capsaicin preparation for the treatment of painful cutaneous disorders and neural dysfunction. The preparation contains a nonionic, amphoteric or cationic surfactant in an amount effective to eliminate or substantially ameliorate burning pain caused by capsaicin.

U.S. Pat. No. 6,573,302 discloses a cream comprising: a topical carrier wherein the topical carrier comprises a member selected from the group comprising lavender oil, myristal myristate, and other preservatives including, hypericum perforatum arnica montana capric acid; and 0.01 to 1.0 wt. % capsaicin; 2 to 10 wt. % an encapsulation agent selected from the group comprising colloidal oatmeal hydrogenated lecithin, dipotassium glycyrlhizinate and combinations thereof; esters of amino acid; a light scattering element having a particle size up to 100 nm.; and a histidine.

U.S. Pat. No. 6,348,501 discloses a lotion for treating the symptoms of arthritis using capsaicin and an analgesic, and a method for making it.

U.S. Pat. No. 5,962,532 discloses methods and compositions for treating pain at a specific site with an effective concentration of capsaicin or analogues. The methods involve providing anesthesia to the site where the capsaicin or analogues thereof is to be administered, and then administering an effective concentration of capsaicin to the joint. The anesthesia can be provided directly to the site, or at remote site that causes anesthesia at the site where the capsaicin is to be administered. For example, epidural regional anesthesia can be provided to patients to which the capsaicin is to be administered at a site located from the waist down. By pretreating the site with the anesthetic, a significantly higher concentration of capsaicin can be used. Effective concentrations of capsaicin or analogues thereof range from between 0.01 and 10% by weight, preferably between 1 and 7.5% by weight, and more preferably, about 5% by weight. This provides for greater and more prolonged pain relief, for periods of time ranging from one week to several weeks. In some cases the pain relief may be more sustained because the disease that underlies the pain may improve due to a variety of factors including enhancement of physical therapy due to less pain in the soft tissues which may foster enhanced mobilization of soft tissues, tendons, and joints.

U.S. Pat. No. 6,054,451 discloses the analgesic composition comprising (R) or (S)-5-(2-azetidinylmethoxy)-2-chloropyridine, or their salts; and an analgesic-potentiating amount of at least one nontoxic N-methyl-D-aspartate receptor antagonist for alleviating pain e.g. arthritic, lumbosacral or musculoskeletal pain or pain associated with a sore throat. It has been claimed that reduced dosages of analgesic are required. U.S. Pat. No. 6,007,841 discloses analgesic composition comprises at least one narcotic agonist-antagonist analgesic and a narcotic agonist-antagonist analgesic-potentiating amount of at least one N-methyl-D-aspartate receptor antagonist.

U.S. Pat. No. 5,516,803 discloses a composition comprising a tramadol material and a nonsteroidal antiinflammatory drug, and its use. The compositions are pharmacologically useful in treating pain and tussive conditions. The compositions are also subject to less opioid side-effects such as abuse liability, tolerance, constipation and respiratory depression. Furthermore, where the components of the compositions are within certain ratios the pharmacological effects of the compositions are superadditive (synergistic).

U.S. Pat. No. 5,336,691 discloses a composition comprising a tramadol material and acetaminophen, and its use. As used herein tramadol refers to various forms of tramadol. The compositions are pharmacologically useful in treating pain and tussive conditions. The compositions are also subject to less opioid side-effects such as abuse liability, tolerance, constipation and respiratory depression. Furthermore, where the components of the compositions are within certain ratios the pharmacological effects of the compositions are superadditive (synergistic).

U.S. Pat. No. 5,919,826 discloses the analgesic effectiveness of tramadol significantly enhanced by administering tramadol with the administration of an analgesia-enhancer which is a nontoxic NMDA receptor blocker and/or a nontoxic substance that blocks at least one major intracellular consequence of NMDA receptor activation for treating arthritis.

U.S. Pat. Nos. 4,656,177 and 4,777,174 disclose combinations of non-narcotic analgesics/nonsteroidal anti-inflammatory drugs and/or narcotic analgesics and caffeine. The compositions elicit a more potent and more rapid analgesic response than if the pain reliever is given alone.

U.S. Pat. No. 5,248,678 teaches a method of increasing the arousal and alertness of comatose patients or nea-comatose patients comprising administering to the patients effective amounts of an adenosine receptor antagonist, such as caffeine, and a GABA agonist, such as gabapentin.

U.S. Pat. No. 6,326,374 discloses compositions that comprise a GABA analog, such as gabapentin or pregabalin in combination with caffeine for the treatment of pain in mammals.

U.S. Pat. No. 6,001,876 discloses a method of using certain analogs of glutamic acid and gamma-aminobutyric acid in pain therapy

U.S. Pat. No. 6,187,338 discloses composition containing (a) neuropathic pain-alleviating amount of at least one anticonvulsant, (b) an anticonvulsant-potentiating amount of at least one nontoxic antagonist for the NMDA receptor or nontoxic substance that blocks a major intracellular consequence of NMDA receptor activation, and a therapeutically effective amount of at (c) least one analgesic. The analgesic is at least one member selected from the group consisting of acetaminophen, aspirin, diclofenac, diflusinal, etodolac, fenbufen, fenoprofen, flufenisal, flurbiprofen, ibuprofen, indomethacin, ketoprofen, ketoro ac, meclofenamic acid, mefenamic acid, nabumetone, naproxen, oxaprozin, phenylbutazone, piroxicam, sulindac, tolmetin and zomepirac.

U.S. Pat. No. 6,242,488 discloses a method for preventing or treating pain in a mammal comprising administering an effective amount of a composition comprising a GABA analog and a non-steroidal anti-inflammatory drug together with a pharmaceutically acceptable excipient, carrier, or diluent thereof.

U.S. Pat. No. 6,242,488 discloses GABA analogs that are useful to prevent and treat gastrointestinal damage and ethanol withdrawal syndrome. Preferred treatments employ gabapentin or pregabalin. Typical compounds include (1-aminomethyl-3-methylcyclohexyl)acetic acid, (1-aminomethyl-3-methylcyclopentyl)acetic acid, (S)-3-(aminomethyl)-5-methylhexanoic acid, 3-aminomethyl-5-methyl-hexanoic acid. and (1-aminomethyl-3,4-dimethylcyclopentyl)acetic acid.

U.S. Pat. No. 6,406,716 discloses the effectiveness of an anticonvulsant such as gabapentin for alleviating neuropathic pain which is potentiated by a nontoxic antagonist for the N-methyl-D-aspartate receptor or nontoxic substance that blocks a major intracellular consequence of N-methyl-D-aspartate receptor activation.

U.S. Pat. No. 6,417,184 discloses a triple drug therapy, pharmaceutical kit, composition, and method of treatment regimen utilized as a combination of effective amounts of an anxiolytic agent, centrally acting alpha anti-adrenergic agent, and central nervous system stimulant for the reduction or prevention of dizziness, drowsiness, depression, delirium, lethargy, mania, orthostatic hypotension, restlessness, weakness in the extremities, and difficulty in being mobile negative side effects caused by therapeutic agents utilized in the treatment of acute and chronic pain syndromes.

US application 20070087977 discloses a pharmaceutical composition comprising an analgesic agent, a blood brain barrier (BBB) transport protein activator and a pharmaceutically acceptable excipient, wherein the analgesic agent is present in an amount sufficient to produce an analgesic effect, and wherein the BBB transport protein activator is present in an amount sufficient to reduce a central nervous system (CNS) effect of the analgesic agent.

US application 20070042969 discloses a method for treating pain in painful diabetic neuropathy which comprises administering in combination a first agent that comprises a compound as defined, illustratively lacosamide, and a second agent effective to provide enhanced treatment of pain, by comparison with the first agent alone. The second agent illustratively comprises an analgesic, an anticonvulsant, an antidepressant or an NMDA receptor antagonist.

US application 20060264509 discloses a method for using α₂δ subunit calcium channel modulators or other compounds that interact with the α₂δ calcium channel subunit in combination with one or more compounds with smooth muscle modulatory effects to treat pain. According to application, α₂δ subunit calcium channel modulators include GABA analogs (e.g., gabapentin and pregabalin), fused bicyclic or tricyclic amino acid analogs of gabapentin, and amino acid compounds. Compounds with smooth muscle modulatory effects include antimuscarinics, β3 adrenergic agonists, spasmolytics, neurokinin receptor antagonists, bradykinin receptor antagonists, and nitric oxide donors.

US Patent Application Publication 20060159743 provides a method of treating a patient suffering from a pain state by administering to the patient a gastric retentive dosage form of gabapentin that is capable of administration in once-daily or twice daily dosing regimens. By reducing the need to administer gabapentin from the thrice-daily administrations characteristic of immediate release gabapentin, the gastric retentive gabapentin dosage forms provided herein have the advantages of improving patient compliance for gabapentin treatment. In addition to the foregoing, the gastric retentive gabapentin dosages forms also exhibit decreased blood plasma concentrations and increased bioavailability throughout the dosing regimen.

US Patent Application Publication 20050009916 discloses a treatment for central neuropathic pain with an analgesic composition that consists essentially of an N-methyl-D-aspartate (NMDA) receptor antagonist. In one embodiment, the application includes chronic administration of the (NMDA) receptor antagonist. In another embodiment, the application is the use of an NMDA receptor antagonist or component thereof for the manufacture of a medicament than includes an analgesic component that consists essentially of an NMDA receptor antagonist for the chronic treatment of central neuropathic pain.

US Patent Application Publication 20060009478 discloses methods and materials, including novel compositions, dosage forms and methods of administration, useful for treating back pain using opioid antagonists, including combinations of opioid antagonists and opioid agonists. Methods and materials comprising opioid antagonists or combinations opioid antagonists and agonists may optionally include one or more additional therapeutic agents.

US Patent Application Publication 20050209319 discloses methods and compositions for treating or preventing local pain or discomfort, particularly local neuropathic pain via topical application directly to skin or mucosal tissue at the site of pain or discomfort are disclosed. Compositions comprising prodrugs of gamma amino butyric acid analogs, such as prodrugs of gabapentin or pregabalin, and optionally a topical anesthetic agent are also disclosed.

US Patent Application Publication 20010008889 discloses the analgesic effectiveness of tramadol is significantly enhanced by administering tramadol prior to, with or following the administration of an analgesia enhancer which is a nontoxic NMDA receptor blocker and/or a nontoxic substance that blocks at least one major intracellular consequence of NMDA receptor activation.

US Patent Application Publication 20010036943 discloses pharmaceutical compositions for the treatment of acute, chronic and/or neuropathic pain. The pharmaceutical compositions are comprised of a therapeutically effective combination of a nicotine receptor partial agonist and an analgesic agent and a pharmaceutically acceptable carrier. The analgesic agent is selected from opioid analgesics, NMDA antagonists, substance P antagonists, COX 1 and COX-2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, and botulinum toxin. The method of using these compounds and a method of treating acute, chronic and/or neuropathic pain and migraine in a mammal including a human is also disclosed.

US Patent Application Publication 20020058656 discloses a triple drug therapy, pharmaceutical kit, composition, and method of treatment regimen utilized as a combination of effective amounts of an anxiolytic agent, centrally acting alpha anti-adrenergic agent, and central nervous system stimulant for the reduction or prevention of dizziness, drowsiness, depression, delirium, lethargy, mania, orthostatic hypotension, restlessness, weakness in the extremities, and difficulty in being mobile negative side effects caused by therapeutic agents utilized in the treatment of acute and chronic pain syndromes.

US Patent Application Publication 20030133951 discloses pharmaceutical compositions for the treatment of acute, chronic and/or neuropathic pain. The pharmaceutical compositions are comprised of a therapeutically effective combination of a nicotine receptor partial agonist and an analgesic agent and a pharmaceutically acceptable carrier. The analgesic agent is selected from opioid analgesics, NMDA antagonists, substance P antagonists, COX-1 and COX-2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, and botulinum toxin. The method of using these compounds and a method of treating acute, chronic and/or neuropathic pain and migraine in a mammal including a human is also disclosed.

US Patent Application Publication 20040002543 discloses composition and method for treating sinus headache or sinus pains including analogs of glutamic acid and gamma-aminobutyric acid in combination with a decongestant.

US Patent Application Publication 20030232787 discloses a novel combination effective for alleviating pain comprising a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, and pharmaceutical compositions comprising same. The administration of endothelin receptor antagonists in these novel combinations results in an improved reduction in the frequency and severity of pain. The incidence of unwanted side effects can be reduced by these novel combinations in comparison to using higher doses of a single agent treatment to achieve a similar therapeutic effect. The present invention is also directed to methods of using effective amounts of the novel combinations and pharmaceutical compositions thereof to treat pain in mammals, including a human.

US Patent Application Publication 20060241134 relates to the combination of certain active compounds from the acid pump antagonist class and compounds which modify gastrointestinal motility.

US Patent Application Publication 20060240128 pertains to an analgesic composition comprising an analgesic drug in an extended release form in combination with an analgesia-enhancing amount of a nontoxic N-methyl-D-aspartate receptor antagonist in an immediate release form.

US Patent Application Publication 20060178354 relates to the treatment of chronic pain using DHEA or derivatives thereof either alone or in combination with at least one other drug. The application also includes compositions comprising DHEA or a derivative thereof and a second drug.

US Patent Application Publication 20050282859 discloses compositions and methods for treatment of genitourinary disorders (e.g., urge incontinence). The compositions may generally include a dual-acting SNRI-NMDA antagonist (e.g., bicifadine and/or milnacipran). Alternatively, the compositions may generally include an SNRI and an NMDA antagonist. US Patent Application Publication 20050245460 relates to methods and compositions for treating CNS-related disorders. A pharmaceutical composition comprising: (a) an NMDA receptor antagonist; (b) a second agent, wherein said agent is an anti-epileptic drug (AED); and (c) a pharmaceutically acceptable carrier, wherein at least one of said NMDA receptor antagonist or said second agent is provided in an extended release dosage form.

US Patent Application Publications 20050203142 and 20050119194 disclose methods of treating, preventing, modifying and managing various types of pain. Specific methods comprise the administration of an immunomodulatory compound, or a pharmaceutically acceptable salt, solvate, hydrate, stereoisomer, clathrate, or prodrug thereof, alone or in combination with a second active agent and/or surgery, psychological or physical therapy. Pharmaceutical compositions, single unit dosage forms, and kits suitable for use in methods of the invention are also disclosed.

US Patent Application Publication 20050065176 relates to a combination of an α₂δ ligand and an AChE inhibitor for use in therapy, particularly in the treatment of pain, particularly neuropathic pain. Particularly preferred α₂δ ligands are gabapentin and pregabalin. Particularly preferred ACHE inhibitors are donepezil (Aricept™), tacrine (Cognex™), rivastigmine (Exelon™), physostgmine (Synapton™), galantamine (Reminyl), metrifonate (Promem), neostigmine (Prostigmin) and icopezil.

US Patent Application Publication 20050059715 relates to a combination, particularly a synergistic combination, of an α₂δ ligand and a dual serotonin-noradrenaline re-uptake inhibitor (DSNRI) or one or both of a selective serotonin re-uptake inhibitor (SSRI) and a selective noradrenaline re-uptake inhibitor (SNRI), and pharmaceutically acceptable salts thereof, pharmaceutical compositions thereof and their use in the treatment of pain, particularly neuropathic pain.

US Patent Application Publication 20040092522 relates to a combination of an α₂δ ligand and a PDE-V inhibitor for use in therapy, particularly in the curative, prophylactic or palliative treatment of pain, particularly neuropathic pain. Particularly preferred α₂δ ligands are gabapentin and pregabalin. Particularly preferred PDE-V inhibitors are sildenafil, vardenafil and tadalafil.

International patent application publication number WO/2005/102390 relates to a synergistic combination of an α2δ ligand and an NMDA receptor antagonist (NMDA antagonist), suitably having affinity for the NR2B-subtype (NR2B antagonist), useful for the treatment of pain. It also relates to a method for treating pain through the use of effective amounts of synergistic combinations of an α2δ ligand and an NMDA antagonist.

US Patent Application Publication 20040063751 discloses a method of treating, preventing, or inhibiting ALS, in a subject in need of such treatment, inhibition or prevention. The method comprises administering to a subject one or more cyclooxygenase-2 selective inhibitor(s) or isomer(s) or pharmaceutically acceptable salt(s), ester(s), or prodrug(s) thereof, in combination with one or more second drugs, wherein the amount of the cyclooxygenase-2 selective inhibitor(s) or isomer(s) or pharmaceutically acceptable salt(s), ester(s), or prodrug(s) thereof in combination with the amount of second drug(s) constitutes an ALS treatment, inhibition or prevention effective amount.

US Patent Application Publication 20030176505 is directed to novel combinations of anti-epileptic compounds that demonstrate pain alleviating properties, with compounds selected from the group consisting of analgesics, NMDA receptor antagonists, and NSAIDs and pharmaceutical compositions comprising same. It has been discovered that the administration of anti-epileptic compounds that demonstrate pain alleviating properties in these novel combinations results in an improved reduction in the frequency and severity of pain. It is also believed that the incidence of unwanted side effects can be reduced by these novel combinations in comparison to using higher doses of a single agent treatment to achieve a similar therapeutic effect. It is also directed to methods of using effective amounts of the novel pharmaceutical compositions to treat pain in mammals.

US Patent Application Publication 20020115705 is directed to novel combinations of anti-epileptic compounds that demonstrate pain alleviating properties, with compounds selected from the group consisting of analgesics, NMDA receptor antagonists, and NSAIDs and pharmaceutical compositions comprising same. It has been discovered that the administration of anti-epileptic compounds that demonstrate pain alleviating properties in these novel combinations results in an improved reduction in the frequency and severity of pain. It is also believed that the incidence of unwanted side effects can be reduced by these novel combinations in comparison to using higher doses of a single agent treatment to achieve a similar therapeutic effect. It is also directed to methods of using effective amounts of the novel pharmaceutical compositions to treat pain in mammals.

U.S. Pat. No. 6,593,368 and U.S. Pat. No. 6,942,876 disclose novel combinations of anti-epileptic compounds that demonstrate pain alleviating properties, with compounds selected from the group consisting of analgesics, N-methyl-D aspartate (NMDA) receptor antagonists and non-steroidal anti-inflammatory drugs (NSAIDS) and pharmaceutical compositions comprising same. Specifically, the patents disclose a combination of an effective amount of an anti-epileptic compound having pain alleviating properties and a compound which is a NMDA receptor antagonist and another combination, comprising a synergistic amounts of gabapentin and celecoxib in a weight/weight ratio of from 1:50 to 50:1, respectively.

US Patent Application Publication 20050038062 discloses methods and compositions for treating subjects with pain, including neuropathic pain, using opioid antagonists or combinations of opioid antagonists and opioid agonists, including, for example, the amount of an opioid antagonist enhances the neuropathic pain-alleviating potency of an opioid agonist. The agonists in the present neuropathic pain-alleviating compositions include: alfentanil, allylprodine, alphaprodine, anileridine, apomorphine, apocodeine, benzylmorphine, bezitramide, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, narceine, nicomorphine, norlevorphanol, normethadone, nalorphine, normorphine, norpipanone, ohmefentanyl, opium, oxycodone, oxymorphone, papavereturn, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, salts thereof, mixtures of any of the foregoing, mixed mu-agonists/antagonists, mu-antagonist combinations, or others known to those skilled in the art. The opioid antagonists in the compositions include: naltrexone, naloxone, nalmefene, methylnaltrexone, methiodide, nalorphine, naloxonazine, nalide, nalmexone, nalorphine dinicotinate, naltrindole (NTI), naltrindole isothiocyanate, (NTH), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, levorphanol, meptazinol, dezocine, or their pharmacologically effective esters or salts. Preferred opioid antagonists include naltrexone, nalmefene, naloxone, or mixtures thereof. Particularly preferred is nalmefene or naltrexone. Further the composition of the antagonist administered is at least 50 to 100 fold less than the amount of the agonist administered. In a preferred dosage form, the maximum amount of antagonist is less than 1 mg. The composition of opioid antagonists or combinations of opioid antagonists and opioid agonists can further contain an anti-convulsant that is lamotrigine, gabapentin, valproic acid, topiramate, famotodine, phenobarbital, diphenylhydantoin, phenyloin, mephenyloin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenyloin sodium, valproate sodium, clobazam, sulthiame, dilantin, diphenylan, or L-5-hydroxytryptophan. The composition of opioid antagonists or combinations of opioid antagonists and opioid agonists can further contain glutamate receptor antagonist that is that is ketamine, MK801, memantine, dextromethorphan, dextrorphan, LY293558, LY382884, amantadine, agmatine, aptiganel, gavestinel, selfotel, 7-chlorokynurenate, MRZ 2/579, MDL 105,519, riluzole, CPP, AP5, APV, NBQX, CNQX or trans-ACPD.

The composition of opioid antagonists or combinations of opioid antagonists and opioid agonists further comprises a local anesthetic that is bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocalne hydrochloride, procaine hydrochloride, propoxycaine hydrochloride tetracaine, or tetracaine hydrochloride.

US Patent Application Publication 20060240128 and WO application 2004022002 disclose an analgesic composition comprising an analgesic drug in an extended release form in combination with an analgesia-enhancing amount of a nontoxic N-methyl-D-aspartate receptor antagonist in an immediate release form. The nontoxic NMDA receptor antagonist is at least one member selected from the group consisting of dextromethorphan, dextrorphan, memantine, amantidine, d-methadone and their pharmaceutically acceptable salts; or the nontoxic NMDA receptor antagonist is present in an immediate release carrier; or the analgesic drug is selected from the group consisting essentially of non-narcotic analgesics, coal tar analgesics, nonsteroidal anti-inflammatory drugs, gabapentin, substance P antagonists, capsaicin, capsaicinoids, and cyclooxygenase-II (COX II) inhibitors; or the weight ratio of the analgesic drug to the nontoxic NMDA receptor antagonist ranges from about 2:1 to about 1:10; or the weight ratio of the analgesic drug to the nontoxic NMDA receptor antagonist ranges from about 1:1 to about 1:5. The analgesic composition wherein the analgesic drug is an analgesically effective amount of at least one opioid analgesic and the analgesic composition is substantially free of opioid antagonist. The opioid analgesic is at least one member selected from the group consisting of alfentanil, allylprodine, alphaprodine, anileridine, benzylmorphine, bezitramide, buprenorphine, butorphanol, clonitazine, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethymethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papavereturn, pentazocine, phenadoxne, phenomorphan, phenazocine, phenoperidine, piminodine, piritramide, propheptazine, promedol, properidine, propoxyphene, sufentanyl, tilidine, tramadol and their pharmaceutically acceptable salts.

US Patent Application Publication 20060167032 discloses the treatment of disorders of the ventral nervous system (CNS) by the administration of a GABA analog such as gabapentin or pregablin, an NMDA receptor antagonist such as dextromethorphan or d-methodone and, optionally, another pharmacologically active substance, e.g., one which is effective for the treatment of a CNS disorder. The pharmaceutical composition contains a therapeutically effective amount of at least one other pharmaceutically active substance (c) which is a drug or drug combination for the treatment of a CNS disorder selected from the group consisting of nicotine, nicotinic compounds, tacrine, donezepil, carbidopa in combination with levodopa, selegiline, bromocriptine, haloperidol, clonidine, pimozide, fluphenazine, benzodiazepines, clonazepam, clorpromazine, fluoxetine, clomipramine, amitriptyline, nortriptyline, imipramine, buspirone, bupropion hydrochloride, venlafaxine, milnacipran, duloxetine, mirtazapine, nefazodone, paroxetine, sertraline, riluzole, trazodone, doxepin and methylphenidate. The CNS disorder is presenile dementia, senile dementia, movement disorder, hyperkinesias, mania, attention deficit disorder, depression, anxiety, obsessive-compulsive disorder, dyslexia, schizophrenia, headache disorder, epilepsy, Tourette's syndrome or Asperger's syndrome.

WO application 2004/089343 discloses water-soluble tablets that dissolve to form clear aqueous solutions, and processes for their preparation. The process includes compressing a mixture of (a) at least one water-soluble active ingredient; (b) one or more water soluble sugar alcohols; (c) one or more water-soluble lubricants; and (d) one or more pH modifiers. The tablet dissolves in about 3 minutes in about 30 ml of water to give a clear solution. The one or more water-soluble active ingredients may be metformin hydrochloride, gabapentin, glibenclamide, glipizide, diltiazem hydrochloride, verapamil hydrochloride, bupropion hydrochloride, propranolol hydrochloride, dextromethorphan hydrobromide, diphenhydramine hydrochloride, disopyramide hydrochloride, tramadol, fluoxetine hydrochloride, paroxetine hydrochloride, pentoxifylline hydrochloride, and diclofenac sodium. The one or more water soluble sugar alcohols may be one or more of sorbitol, mannitol, spray dried mannitol, xylitol, erythritol isomalt and hydrogenated starch hydrolysates and combinations thereof. The one or more water-soluble lubricants may be one or more of DL-leucine, sodium lauryl sulphate, magnesium lauryl sulphate and polyethylene glycol. The one or more pH modifiers may be one or more of potassium hydroxide, sodium hydroxide, monosodium citrate, citric acid and the like. While the patent application claims the process for water soluble tablets are novel, U.S. Pat. Nos. 4,347,235 and 3,976,601 discloses such process for making such water soluble tablets.

U.S. Pat. No. 5,910,512 discloses a water-based topical analgesic and method of application wherein the analgesic contains capsicum, capsicum oleoresin and/or capsaicin. This analgesic is applied to the skin to provide relief for rheumatoid arthritis, osteoarthritis, and the like.

U.S. Pat. No. 5,403,868 discloses novel capsaicin derivatives containing thio-urea, processes for the production thereof, pharmaceutical compositions containing them and use thereof as pharmaceuticals.

U.S. Pat. No. 5,178,879 discloses clear, water-washable, non-greasy gels useful for topical pain relief contain capsaicin, water, alcohol and a carboxypolymethylene emulsifier. A method of preparing the gels is also disclosed

U.S. Pat. No. 5,021,450 relates to a new class of compounds having a variable spectrum of activities for capsaicin-like responses, compositions thereof, processes for preparing the same, and uses thereof. Compounds were prepared by combining phorbol related diterpenses and homovanillac acid analogs via esterification at the exocyclic hydroxy group of the diterpene. Examples of these compounds include 20-homovanillyl-mezerein and 20-homovanillyl-12-deoxyphorbol-13-phenylacetate.

U.S. Pat. No. 4,997,853 discloses a method and composition for treating superficial pain syndromes which incorporates capsaicin in a therapeutically effective amount into a pharmaceutically acceptable carrier and adding to this composition a local anesthetic such as lidocaine or benzocaine. The composition containing the anesthetic is then applied to the site of the pain. A variation on the treatment includes initial treatment with the composition containing the local anesthetic until the patient has become desensitized to the presence of capsaicin and subsequent treatment with a composition omitting the local anesthetic.

US Patent Application Publication 20050019436 provides compositions and methods for relieving pain at a site in a human or animal in need thereof by administering at a discrete site in a human or animal in need thereof a dose of capsaicin in an amount effective to denervate a discrete site without eliciting an effect outside the discrete location, the dose of capsaicin ranging from 1 μg to 3000 μg.

US Patent Application Publication 20040224037 claims a use of Capsaicin (8-methyl-n-vanillyl-6-nonenamide), its derivatives, vanilloids and capsicum extract, to combat and control HIV (humans immunodeficiency virus) and aids (acquired immunodeficiency syndrome). An evaluation of a capsicum sp consumption of a long term aids survivors group permitted a definition of more efficacious ways to administer the substance. capsaicin intravenous and by subcutaneous or intramuscular administration at low concentration implemented by using infuses, it inhibits HIV replication and stimulates the production and proliferation of lymphocytes and cells nk. Also it acts as disinfectant in macrophages, and has a power as anticancer and antioxidant agent. Moreover has the property to control and annihilate common opportunistic illnesses related to HIV due to its triple antibiotic characteristics.

US Patent Application Publication 20040146590 provides methods and kits for the selective ablation of pain-sensing neurons. The methods comprise administration of a vanilloid receptor agonist to a ganglion in an amount that causes death of vanilloid receptor-bearing neurons. Accordingly, the present invention provides methods of controlling pain and inflammatory disorders that involve activation of vanilloid receptor-bearing neurons.

US Patent Application Publication 20030133995 discloses a chemical composition for an ingestible capsaicin neutralizer to neutralize the effect of capsaicin on the oral cavity, tongue, and esophagus when capsaicin from hot peppers is ingested by a user comprised of an effective neutralizing amount of casein protein, or the salt thereof, an alkali earth metal halide, and the balance water.

US Patent Application Publication 20030082249 discloses a composition for use in treating or preventing mucositis, and/or xerostomia, including capsaicin or capsaicin derivative, and one or more additional compounds useful in treating mucositis and/or xerostomia, wherein the composition is provided in an oral delivery vehicle. The term capsaicin derivative and capsaicinoid as used in the disclosure are interchangeable and generally refer to capsaicin analogs. Among the capsaicinoids useful in the practice of the disclosure are capsaicin, capsaicin derivatives; dihydrocapsaicin; norhydrocapsaicin; nordihydrocapsaicin; homocapsaicin; homohydrocapsaicin; homodihydrocapsaicin; civamide (cis-capsaicin); nonivamide; NE-19550 (N-[4-hydroxy-3-methoxyphenyl)methyl-l]-9Z-octadecanamide) (olvanil); NE-21610 (N-[(4-(2-aminoethoxy)-3-methoxyphenyl)methyl]-9Z-octadecanamide) Sandoz Pharmaceutical Corp, East Hanover, N.J.); NE-28345 (N-(9Z-octadecenyl)-3-methoxy-4-hydroxyphenylacetamide; also known as N-oleyl-homovanillamide); and their analogs and derivatives (U.S. Pat. No. 5,762,963, which is incorporated herein by reference). NE-19550, NE-21610, and NE-28345 are discussed in Dray et al, (1990).

US Patent Application Publication 20020058048 discloses a topical capsaicin preparation for the treatment of painful cutaneous disorders and neural dysfunction is disclosed. The preparation contains a nonionic, amphoteric or cationic surfactant in an amount effective to eliminate or substantially ameliorate burning pain caused by capsaicin.

US Patent Application Publication 20010002406 discloses transdermal application of capsaicin (or a capsaicin analog) in a concentration from greater than about 5% to about 10% by weight to be an extremely effective therapy for treating neuropathic pain, so long as an anesthetic, preferably by means of a transdermal patch, is administered initially to the affected area to minimize the expected side effects from subsequent capsaicin application. Analogs of capsaicin with physiological properties similar to capsaicin are known (Ton 1955). For example, resiniferatoxin is described as a capsaicin analog by Blumberg, U.S. Pat. No. 5,290,816. U.S. Pat. No. 4,812,446, describes capsaicin analogs and methods for their preparation

U.S. Pat. No. 7,157,103 discloses an oral dosage form comprising a therapeutically effective amount of a drug susceptible to abuse; and an effective amount of an irritant to impart an irritating sensation to an abuser upon administration of said dosage form after tampering.

US Patent Application Publication 20060240128 discloses a combined analgesic composition having at least one analgesic drug in an extended release form and at least one nontoxic N-methyl-D-aspartate receptor antagonist in an immediate release form, where the activity of the analgesic drug is enhanced by the at least one nontoxic N-methyl-D-aspartate receptor antagonist. Preferably, the analgesic drug is an opioid analgesic, the at least one nontoxic N-methyl-D-aspartate receptor antagonist is dextromethorphan, and the analgesic composition is substantially free of opioid antagonist.

US Patent Application Publication 20030064122 discloses pharmaceutical compositions which include systems to deter abuse. More specifically, the disclosure relates to compositions containing an effective amount of pharmaceutical compound and capsaicin or a capsaicinoid compound. Most specifically, the disclosure relates to a composition containing an effective amount of a pharmaceutical compound, and an amount of a capsaicin compound to deter intranasal, oral, and intravenous abuse while having little or no irritating effect when administered orally or transdermally as directed. The application claims a composition comprising: a pharmaceutically active ingredient; a capsaicinoid; wherein said composition is for subsequent formulation into a final dosage form selected from a solid oral dosage form and a transdermal dosage form; and wherein said capsaicinoid is present in an amount such that said final dosage form contains an amount effective to cause at least one response selected from coughing, sneezing, secretion, and pain when contacted with a mucosal or vascular membrane.

US Patent Application Publication 20020035105 describes the neuropathic pain alleviating effectiveness of an antidepressant is significantly potentiated by administering the antidepressant prior to, with or following the administration of a nontoxic NMDA receptor antagonist.

U.S. Pat. Nos. 4,493,848 and 4,564,633 disclose the derivatives of capsaicin, including short chain ester derivatives (C1-C6) of capsaicin for relieving pain.

U.S. Pat. No. 7,244,767 relates to the use of N-acylvanillinamide derivatives capable of activating the peripheral receptor CB1 of cannabinoids. In particular, the present invention relates to the use of compounds for the preparation of a medicinal product which is capable of activating the peripheral receptor CB1 of cannabinoids.

Heretofore, there has been no recognition or appreciation that the analgesic effectiveness of tramadol can be appreciably enhanced by administration of tramadol prior to, with or following the administration of an analgesia-enhancing amount of dextromethorphan or magnesium or for that matter, any other NMDA receptor antagonist and an anticonvulsant and/or a tricyclic anti-depressant.

The inventor was searching for a synergistic combination for the total relief of pain associated with fibromyalgia syndrome, diabetic neuropathy, multiple sclerosis and cancer. Surprisingly, it has now been found, in a highly unexpected fashion, that a combination of a non-toxic NMDA receptor antagonist magnesium with tramadol or its analog and an anticonvulsant and/or a tricyclic anti-depressant exhibits significant palliative effects on these types of chronic pain. Further it has been found in a highly unexpected fashion that such a combination can have a capsaicin or an ester of capsaicin with added benefit.

Accordingly, an object of the invention is to provide methods and compositions for the treatment of acute or chronic pain which provide effective control of pain without the harmful side effects associated with traditional analgesics, such as respiratory depression, disturbed sleep patterns, diminished appetite, seizures, and psychological and/or physical dependency.

In addition, the present invention can avoid the liability of gastrointestinal and liver toxicity by omitting acetaminophen, aspirin and other NSAID's. Acetaminophen toxicity is well known and represents a significant drawback of all formulations that contain it. The limiting dose of acetaminophen is on the order of 2 grams per day. It has also been determined that intentional overdose of acetaminophen is the second most common method of committing suicide in Europe. Thus, reducing or eliminating exposure to acetaminophen is of significant importance.

These and other objects and features of the invention will be apparent from the following description.

### SUMMARY

It is an object of the present invention to provide a method and pharmaceutical formulation, (medicament), which allows for reduced plasma concentrations of active ingredients, while still providing effective pain management.

It is a further object of the present invention to provide a method and a pharmaceutical formulation (medicament) for effectively treating patients in pain. Accordingly, the present invention provides a method that comprises administering a pharmaceutical composition comprising an analgesic combination that includes a NMDA receptor antagonist or a pharmaceutically acceptable salt thereof, an anticonvulsant and/or a tricyclic anti-depressant or a pharmaceutically acceptable salt thereof, and tramadol or its analog, or a pharmaceutically acceptable salt thereof. The pharmaceutical formulation can further contain capsaicin or an ester of capsaicin. By this method is achieved an analgesic preparation which produces prolonged and effective pain management, while at the same time exhibits reduced side effects and decreases the liability to dependence and tolerance which the patients may experience when subjected to prolonged treatment with an opiate.

In accordance with the present invention, a NMDA receptor antagonist can be dextromethorphan, magnesium, dextrorphan, ketamine, amantadine, memantine, eliprodil, ifenprodil, phencyclidine, MK-801, dizocilpine, CCPene, flupirtine, or derivatives or salts thereof. Even though magnesium exerts various physiological effects, for the purpose of teaching the present invention, it will be simply be referred as NMDA receptor antagonist.

An anticonvulsant can be, for example, gabapentin, pregabalin, 3-methyl gabapentin or derivatives thereof.

A tramadol or its analog can be any one of (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-desmethyl tramadol”), venlafaxine, (R/S)-1-[2-(dimethylamino)-1-(4-methoxyphenyl)ethyl]cyclohexanol and O-desmethylvenlafaxine or mixtures, stereoisomers or recemates thereof.

A capsaicin can be capsaicin itself, civamide, homocapsaicin, nordihydrocapsaicin, dihydrocapsaicin, homodihydrocapsaicin, n-vanillyloctanamide, nonivamide, n-vanillyldecanamide, cis-capsaicin, or derivatives thereof (see FIG. 3).

The term “ester derivatives of capsaicin” or “ester of capsaicin” in the present invention refers to the acylated derivatives of capsaicin and is denoted by the formula I (see FIG. 3). The pharmaceutical composition and their utilities have been disclosed in a different patent application. These derivatives are capable of reverting to the active parent compound following enzymatic or chemical hydrolysis. These derivatives have a higher lipophilicity, lipid solubility and less irritation to the skin than the parent compound, and hence are better able to be incorporated into certain pharmaceutical formulations, including cream and ointment pharmaceutical formulations. The compounds of the present invention are set forth by the following formula:

R—CO-CAP (I)

wherein CAP refers to collectively the capsaicins represented in FIG. 3.

In formula I, R is selected from alkyl groups of up to about 22 carbon atoms and aryl groups of up to about 22 carbon atoms and alkylene group of up to about 22 carbon atoms and an arylene group of up to about 22 carbon atoms. The alkyl, aryl and alkylene groups may be substituted or unsubstituted, branched or straight chains. In addition, R may contain heteroatoms and may be straight chained or branched.

Examples of suitable straight-chain alkyl groups in formula I include methyl, ethyl, propyl, butyl, hexyl, heptyl, octyl, dodecyl, 1-pentadecyl, 1-heptadecyl and the like groups.

Examples of suitable branched chain alkyl groups in formula I include isopropyl, sec-butyl, t-butyl, 2-methylbutyl, 2-pentyl, 3-pentyl and the like groups.

Examples of suitable cyclic alkyl groups in formula I include cyclopropyl, cyclobutyl, cyclopentyl and cyclohexyl groups.

Examples of suitable “alkenyl” groups in I include vinyl (ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” in formula I are pyridinyl, thienyl or imidazolyl.

As noted herein, the expression “halo” is meant in the conventional sense to include F, Cl, Br, and I.

Among the compounds represented by the general Formula I, preferred compounds are such in which R is one of the following groups: methyl, ethyl, propyl, butyl, pentyl, hexyl, 1-pentadecyl, 1-heptadecyl, isobutyl, methoxyethyl, ethoxyethyl, benzyl and nicotinyl. The compounds of Formula I are esters of capsaicin.

The present invention further provides a method and composition for effectively treating patients in pain which avoids the toxicities associated with NSAID or acetaminophen therapy. The method comprises administering a pharmaceutical composition to a patient in need of treatment for pain, wherein the pharmaceutical composition comprises an analgesic combination comprising a NMDA antagonist or a pharmaceutically acceptable salt thereof, an anticonvulsant and/or a tricyclic anti-depressant or a pharmaceutically acceptable salt thereof, and tramadol or its analog, or a pharmaceutically acceptable salt thereof. The composition can further contain capsaicin or an ester of capsaicin.

In accordance with the present invention, the composition can be essentially free of a NSAID or acetaminophen. Particularly relevant NSAIDs include ibuprofen, diclofenac, diflunisal, etodolac, fenoprofen, flurbiprofen, indomethacin, ketoprofen, ketorolac, mefenamic acid, meclofenamate, nabumetone, naproxen, oxaprozin or piroxicam. If the patient is separately administered a NSAID and/or acetaminophen, the amount administered is not enough to induce one or more toxicities associated with the use of the NSAID and/or acetaminophen.

Although tramadol/acetaminophen formulations containing a slew of other pharmaceutically active agents such as decongestants, antitussives, antihistamines or suspected adjuvants have been suggested in a general fashion, the particular combination of NMDA receptor antagonist, tramadol or its analog and anticonvulsant and/or a tricyclic anti-depressant has not been previously recognized or appreciated. Similarly, the particular combination of NMDA receptor antagonist, tramadol or its analog, capsaicin or an ester of capsaicin and anticonvulsant and/or a tricyclic anti-depressant in a composition has not been recognized or appreciated. Similarly, the particular combination of NMDA receptor antagonist, tramadol or its analog and anticonvulsant and/or a tricyclic anti-depressant in a composition essentially free of a NSAID and/or acetaminophen has not been recognized or appreciated. Similarly, the particular combination of NMDA receptor antagonist, tramadol or its analog, capsaicin or an ester of capsaicin and anticonvulsant and/or a tricyclic anti-depressant in a composition essentially free of a NSAID and/or acetaminophen has not been recognized or appreciated.

In accordance with the present invention, the ratio of NMDA antagonist to tramadol or its analog can be from about 15:1 to 1:15, about 10:1 to 1:10, about 5:1 to 1:5, or about 1:2 to 1:2. The ratio of NMDA antagonist to anticonvulsant and/or a tricyclic anti-depressant to tramadol or its analog can be from about 90:1:1 to 1:90:1 to 1:1:90, preferably from about 10:1:1 to 1:10:1 to 1:1:10 and more preferably from 3:1:1 to 1:3:1 to 1:1:3. In a similar fashion, the ratio of NMDA antagonist to anticonvulsant and/or a tricyclic anti-depressant to tramadol or its analog to capsaicin or an ester of capsaicin can be from about 90:1:1:1 to 1:90:1:1 to 1:1:90:1 to 1:1:1:90 and preferably from about 10:1:1:1 to 1:10:1:1 to 1:1:10:1 to 1:1:1:10.

It is yet a further object to provide a method and pharmaceutical formulation (medicament) for the effective treatment of pain in patients by augmenting the analgesic effect of tramadol or its analog.

The invention is directed to the surprising and unexpected synergy obtained via the administration of a NMDA receptor antagonist together with an anticonvulsant and/or a tricyclic anti-depressant and tramadol or its analog. This synergy can be further augmented by the addition of capsaicin or an ester of capsaicin.

The present invention is related in part to: a) analgesic pharmaceutical compositions comprising a NMDA receptor antagonist together with an anticonvulsant and/or a tricyclic anti-depressant and tramadol or its analog; b) analgesic pharmaceutical compositions comprising a NMDA receptor antagonist together with an anticonvulsant and/or a tricyclic anti-depressant, tramadol or its analog and capsaicin or an ester of capsaicin. The pharmaceutical compositions can be administered intravenously, intrathecally, orally, via controlled release implant or pump, parenterally, sublingually, rectally, topically, via inhalation, etc. In other embodiments of the invention, tramadol or its analog can be administered separately from the NMDA receptor antagonist, capsaicin or an ester of capsaicin and the anticonvulsant and/or a tricyclic anti-depressant, as set forth in more detail below.

The invention allows for the use of lower doses of a tramadol or its analog or a NMDA receptor antagonist, (referred to as apparent “one-way synergy” herein), or lower doses of both drugs (referred to as “two-way synergy” herein) than would normally be required when either drug is used alone. By using lower amounts of either or both drugs, the side effects associated with effective pain management in humans and other species are significantly reduced.

In certain preferred embodiments, the invention is directed in part to synergistic combinations of dextromethorphan or other NMDA receptor antagonist in an amount sufficient to render a therapeutic effect together with an anticonvulsant and/or a tricyclic anti-depressant and tramadol or its analog, such that an analgesic effect is attained which is at least about 5 (and preferably at least about 10) times greater than that obtained with the dose of tramadol or its analog alone. This is exemplified by the apparent fact that patients with diabetic neuropathy and fibromyalgia, who could not get even 30-40% reduction in pain even with the administration of 400 mg of tramadol per day, can have shown 90-100% pain relief with the inventive composition containing 35 mg of tramadol, 45 mg of dextromethorphan and 90 mg of gabapentin over a period of 12-16 hours.

In certain embodiments, the synergistic combination provides an analgesic effect which is up to about 10 to 20 times greater than that obtained with the dose of an anticonvulsant and/or a tricyclic anti-depressant if administered as a single agent. In such embodiments, the synergistic combinations display what is referred to herein as an “apparent mutual synergy”, meaning that the dose of NMDA antagonist and anticonvulsant and/or a tricyclic anti-depressant synergistically potentiates the effect of tramadol or its analog and the dose of tramadol or its analog appears to potentiate the effect of the NMDA antagonist and the anticonvulsant and/or a tricyclic anti-depressant.

The combination of NMDA antagonist, anticonvulsant and/or a tricyclic anti-depressant and tramadol or its analog can be administered in a single dosage form. Similarly, the combination of NMDA antagonist, anticonvulsant and/or a tricyclic anti-depressant, capsaicin or an ester of capsaicin and tramadol or its analog can be administered in a single dosage form. Alternatively the combination can be administered separately, preferably concomitantly.

In certain preferred embodiments, the synergism exhibited between the three types of drugs, is such that the dosage of tramadol or its analog would be sub-therapeutic if administered without the dosage of the NMDA antagonist and anticonvulsant and/or a tricyclic anti-depressant. This synergy can be further augmented by the addition of a fourth drug, capsaicin or an ester of capsaicin. Similarly, in certain preferred embodiments wherein the pharmaceutical composition comprises a combination of NMDA antagonist, anticonvulsant and/or a tricyclic anti-depressant and tramadol or its analog and is essentially free of a NSAID or acetaminophen, the dosage of tramadol or its analog would be sub-therapeutic if administered without the dosage of the NMDA antagonist and anticonvulsant and/or a tricyclic anti-depressant. In other preferred embodiments, the present invention relates to a pharmaceutical composition comprising an analgesically effective dose of tramadol or its analog together with a dose of a NMDA antagonist and an anticonvulsant and/or a tricyclic anti-depressant effective to augment the analgesic effect of tramadol or its analog, or a composition essentially free of a NSAID or acetaminophen and comprising an analgesically effective dose of tramadol or its analog together with a dose of a NMDA antagonist effective to augment the analgesic effect of tramadol or its analog

It is believed that in actuality these combinations exhibit two-way synergism, meaning that the NMDA antagonist and the anticonvulsant and/or a tricyclic anti-depressant potentiate the effect of tramadol or its analog, and tramadol or its analog, potentiates the effect of the NMDA antagonist and the anticonvulsant and/or a tricyclic anti-depressant. This synergy can be further augmented by the addition of a fourth drug, capsaicin or an ester of capsaicin. Thus, other embodiments of the invention relate to combinations of NMDA antagonist, anticonvulsant and/or a tricyclic anti-depressant and tramadol or its analog where the dose of each drug is reduced due to the synergism demonstrated between the drugs, and the analgesia derived from the combination of drugs in reduced doses is surprisingly and strongly enhanced. The two-way synergism is not always readily apparent in actual dosages due to the potency ratio of tramadol or its analog to the NMDA antagonist and anticonvulsant and/or a tricyclic anti-depressant. By this we mean that tramadol or its analog generally displays unexpectedly enhanced analgesic potency.

In certain preferred embodiments, the invention is directed to pharmaceutical formulations comprising a NMDA antagonist such as dextromethorphan and magnesium, an anticonvulsant and/or a tricyclic anti-depressant in an amount sufficient to render a therapeutic effect, and a therapeutically effective or sub-therapeutic amount of tramadol or its analog. Similarly, the invention is directed to pharmaceutical formulations comprising a NMDA antagonist such as dextromethorphan and magnesium, an anticonvulsant and/or a tricyclic anti-depressant, capsaicin or an ester of capsaicin in an amount sufficient to render a therapeutic effect, and a therapeutically effective or sub-therapeutic amount of tramadol or its analog. Preferably, tramadol or its analog is selected from the group consisting of tramadol, its metabolites thereof, salts thereof, recemates thereof, and complexes thereof.

In certain preferred embodiments, the invention is directed to pharmaceutical formulations comprising a NMDA antagonist such as dextromethorphan and magnesium, and an anticonvulsant and/or a tricyclic anti-depressant in an amount sufficient to render a therapeutic effect together with a therapeutically effective or sub-therapeutic amount of tramadol or its analog. Similarly, the invention is directed to pharmaceutical formulations comprising a NMDA antagonist such as dextromethorphan and magnesium, capsaicin or an ester of capsaicin and an anticonvulsant and/or a tricyclic anti-depressant in an amount sufficient to render a therapeutic effect together with a therapeutically effective or sub-therapeutic amount of tramadol or its analog. Preferably, tramadol or its analog is selected from the group consisting of tramadol, its metabolites thereof, salts thereof, recemates thereof, and complexes thereof.

In certain embodiments, the invention is directed to pharmaceutical formulations comprising a NMDA antagonist such as dextromethorphan and magnesium, and an anticonvulsant and/or a tricyclic anti-depressant in an amount sufficient to render a therapeutic effect together with a dose of tramadol or its analog that is analgesic if administered without the NMDA antagonist and the anticonvulsant and/or a tricyclic anti-depressant. Preferably, tramadol or its analog is tramadol. The dose of tramadol is preferably from about 30 to about 400 mg.

The invention further relates to a method of effectively treating pain in mammals or humans, comprising administration to a human or mammalian patient a therapeutically effective amount of a NMDA antagonist and an anticonvulsant and/or a tricyclic anti-depressant together with a dose of tramadol or its analog, such that the combination provides an analgesic effect which is at least about 5, and preferably at least about 10, times greater than that obtained with the dose of tramadol or its analog alone. In certain embodiments, the synergistic combination provides an analgesic effect which is up to about 30 to 40 times greater than that obtained with the dose of tramadol or its analog alone.

In certain preferred embodiments, the doses of the NMDA antagonist, anticonvulsant and/or a tricyclic anti-depressant and tramadol or its analog are administered orally. In further preferred embodiments the doses of the NMDA antagonist, anticonvulsant and/or a tricyclic anti-depressant and tramadol or its analog are administered in a single oral dosage form. In certain preferred embodiments, the dose of tramadol or its analog would be sub-therapeutic if administered without the dose of the NMDA antagonist and the anticonvulsant and/or a tricyclic anti-depressant. In other preferred embodiments, the dose of tramadol or its analog is effective to provide analgesia alone, but the dose of tramadol or its analog provides at least a five fold greater analgesic effect than typically obtained with that dose of tramadol or its analog alone.

The invention further relates to the use of a pharmaceutical combination of a NMDA antagonist(s) together with a tramadol or its analog and an anticonvulsant and/or a tricyclic anti-depressant to provide effective pain management in humans and other mammals. Similarly, the invention further relates to the use of a pharmaceutical combination of a NMDA antagonist(s) together with a tramadol or its analog, an anticonvulsant and/or a tricyclic anti-depressant and capsaicin or an ester of capsaicin to provide effective pain management in humans and other mammals. The instant invention is a method of using a pharmaceutical combination in the treatment of pain, especially for treatment of chronic pain disorders. Such disorders include, but are not limited to, inflammatory pain, postoperative pain, osteoarthritis, pain associated with metastatic cancer, trigeminal neuralgia, acute herpetic and postherpetic neuralgia, diabetic neuropathy, causalgia, brachial plexus avulsion, occipital neuralgia, reflex sympathetic dystrophy, fibromyalgia, gout, phantom limb pain, bum pain, and other forms of neuralgic, neuropathic, and idiopathic pain syndromes.

The invention further relates to the use of a NMDA antagonist in the manufacture of a pharmaceutical preparation containing a NMDA antagonist, an anticonvulsant and/or a tricyclic anti-depressant and tramadol or its analog for the treatment of pain. Similarly, the invention further relates to the use of a NMDA antagonist in the manufacture of a pharmaceutical preparation containing a NMDA antagonist, an anticonvulsant and/or a tricyclic anti-depressant, capsaicin or an ester of capsaicin and tramadol or its analog for the treatment of pain.

The invention further relates to the use of a tramadol or its analog in the manufacture of a pharmaceutical preparation containing a NMDA antagonist, an anticonvulsant and/or a tricyclic anti-depressant, and tramadol or its analog for the treatment of pain of chronic, intermittent or acute nature. Similarly, the invention further relates to the use of a tramadol or its analog in the manufacture of a pharmaceutical preparation containing a NMDA antagonist, an anticonvulsant and/or a tricyclic anti-depressant, capsaicin or an ester of capsaicin and tramadol or its analog for the treatment of pain of chronic, intermittent or acute nature.

The invention further relates to the use of an anticonvulsant and/or a tricyclic anti-depressant in the manufacture of a pharmaceutical preparation containing a NMDA antagonist, an anticonvulsant and/or a tricyclic anti-depressant and tramadol or its analog for the treatment of pain of chronic, intermittent or acute nature. Similarly, the invention further relates to the use of an anticonvulsant and/or a tricyclic anti-depressant in the manufacture of a pharmaceutical preparation containing a NMDA antagonist, an anticonvulsant and/or a tricyclic anti-depressant, capsaicin or an ester of capsaicin and tramadol or its analog for the treatment of pain of chronic, intermittent or acute nature.

The invention further relates to the use of capsaicin or an ester of capsaicin in the manufacture of a pharmaceutical preparation containing a NMDA antagonist, an anticonvulsant and/or a tricyclic anti-depressant, capsaicin or an ester of capsaicin and tramadol or its analog for the treatment of pain of chronic, intermittent or acute nature.

The invention is also directed to a method for providing effective pain management in humans, comprising administration of either an analgesically effective or sub-therapeutic amount of a tramadol or its analog, administration of an effective amount of an anticonvulsant and/or a tricyclic anti-depressant in an amount effective to augment synergistically the analgesic effect provided by said tramadol or its analog, and administration of an effective amount of a NMDA antagonist such as dextromethorphan in an amount effective to augment synergistically the analgesic effect provided by said tramadol or its analog. The NMDA antagonist and anticonvulsant and/or a tricyclic anti-depressant can be administered prior to, concurrently with, or after administration of tramadol or its analog, as long as the dosing interval of NMDA antagonist overlaps with the dosing interval of tramadol or its analog and/or its analgesic effects.

The invention is also directed to a method for providing effective pain management in humans, comprising administration of either an analgesically effective or sub-therapeutic amount of a tramadol or its analog, administration of an effective amount of an anticonvulsant and/or a tricyclic anti-depressant in an amount effective to augment synergistically the analgesic effect provided by said tramadol or its analog, administration of an effective amount of capsaicin or an ester of capsaicin in an amount effective to augment synergistically the analgesic effect provided by said tramadol or its analog and administration of an effective amount of a NMDA antagonist such as dextromethorphan and magnesium, in an amount effective to augment synergistically the analgesic effect provided by said tramadol or its analog. The NMDA antagonist, capsaicin or an ester of capsaicin and anticonvulsant and/or a tricyclic anti-depressant can be administered prior to, concurrently with, or after administration of tramadol or its analog, as long as the dosing interval of NMDA antagonist, capsaicin or an ester of capsaicin and anticonvulsant and/or a tricyclic anti-depressant overlaps with the dosing interval of tramadol or its analog and/or its analgesic effects.

The anticonvulsant and/or a tricyclic anti-depressant can be administered prior to, concurrently with, or after administration of tramadol or its analog and a NMDA antagonist, as long as the dosing interval of the anticonvulsant and/or a tricyclic anti-depressant and a NMDA antagonist overlaps with the dosing interval of tramadol or its analog and/or its analgesic effects. In other words, according to the method of the present invention, in certain preferred embodiments the NMDA antagonist and the anticonvulsant and/or a tricyclic anti-depressant need not be administered in the same dosage form or even by the same route of administration as tramadol or its analog. Rather, the method is directed to the surprising synergistic and/or additive analgesic benefits obtained in humans or other mammals, when analgesically effective levels of tramadol or its analog have been administered to a human or other mammals, and, prior to or during the dosage interval for tramadol or its analog or while the human or other mammal is experiencing analgesia, an effective amount of NMDA antagonist and anticonvulsant and/or a tricyclic anti-depressant to augment the analgesic effect of tramadol or its analog is administered. If the NMDA antagonist and the anticonvulsant and/or a tricyclic anti-depressant are administered prior to the administration of tramadol or its analog, it is preferred that the dosage intervals for the two drugs overlap, i.e., such that the analgesic effect over at least a portion of the dosage interval of tramadol or its analog is at least partly coincident with the period of useful therapeutic effect of the NMDA antagonist and the anticonvulsant and/or a tricyclic anti-depressant. Similarly, if the NMDA antagonist, capsaicin or an ester of capsaicin and the anticonvulsant and/or a tricyclic anti-depressant are administered prior to the administration of tramadol or its analog, it is preferred that the dosage intervals for the three drugs overlap, i.e., such that the analgesic effect over at least a portion of the dosage interval of tramadol or its analog is at least partly coincident with the period of useful therapeutic effect of the NMDA antagonist, capsaicin or an ester of capsaicin and the anticonvulsant and/or a tricyclic anti-depressant.

In an additional method of the invention, the surprising synergistic and/or additive benefits obtained in humans are achieved when analgesically effective levels of a tramadol or its analog have been administered to a human during the time period of the therapeutic effect of a NMDA antagonist and an anticonvulsant and/or a tricyclic anti-depressant. Alternatively the method comprises the effective analgesia obtained when the human or other mammal is experiencing analgesia by virtue of the administration of a NMDA antagonist and an anticonvulsant and/or a tricyclic anti-depressant and an effective amount of a tramadol or its analog to synergistically augment the analgesic effect of tramadol or its analog. Similarly, the surprising synergistic and/or additive benefits obtained in humans are achieved when analgesically effective levels of a tramadol or its analog have been administered to a human during the time period of the therapeutic effect of a NMDA antagonist, capsaicin or an ester of capsaicin and an anticonvulsant and/or a tricyclic anti-depressant. Alternatively the method comprises the effective analgesia obtained when the human or other mammal is experiencing analgesia by virtue of the administration of a NMDA antagonist, capsaicin or an ester of capsaicin and an anticonvulsant and/or a tricyclic anti-depressant and an effective amount of a tramadol or its analog to synergistically augment the analgesic effect of tramadol or its analog.

In a further embodiment of the present invention, the invention comprises an oral solid dosage form comprising an analgesically effective amount of tramadol or its analog together with an amount of a NMDA antagonist and an anticonvulsant and/or a tricyclic anti-depressant which augment the effect of tramadol or its analog. Yet in a further embodiment of the present invention, the invention comprises an oral solid dosage form comprising an analgesically effective amount of tramadol or its analog together with an amount of a NMDA antagonist, capsaicin or an ester of capsaicin and an anticonvulsant and/or a tricyclic anti-depressant which augment the effect of tramadol or its analog.

Optionally, the oral solid dosage form includes a sustained release carrier that effectuates the sustained release of tramadol or its analog, or both the tramadol or its analog and the NMDA antagonist when the dosage form contacts gastrointestinal fluid. The sustained release dosage form may comprise a multiplicity of substrates and carriers that include the drugs. The substrates may comprise matrix spheroids or may comprise inert pharmaceutically acceptable beads that are coated with the drugs. The coated beads are then preferably overcoated with a sustained release coating comprising the sustained release carrier. The matrix spheroid may include the sustained release carrier in the matrix itself, or the matrix may comprise a simple disintegrating or prompt release matrix containing the drugs, the matrix having a coating applied thereon which comprises the sustained release carrier. In yet other embodiments, the oral solid dosage form comprises a tablet core containing the drugs within a normal or prompt release matrix with the tablet core being coated with a sustained release coating comprising the sustained release carrier.

In yet further embodiments, the tablet or capsule contain the drugs within a sustained release matrix comprising the sustained release carrier. In yet further embodiments, the tablet contains tramadol or its analog within a sustained release matrix, and the NMDA antagonist and anticonvulsant and/or a tricyclic anti-depressant coated into the tablet as an immediate release layer. In yet further embodiments, the tablet contains tramadol or its analog within a sustained release matrix, and the NMDA antagonist, capsaicin or an ester of capsaicin and anticonvulsant and/or a tricyclic anti-depressant coated into the tablet as an immediate release layer.

In many preferred embodiments of the invention, the pharmaceutical compositions containing the NMDA antagonist, an anticonvulsant and/or a tricyclic anti-depressant and tramadol or its analog set forth herein are administered orally. Such oral dosage forms may contain one or all of the drugs in immediate or sustained release form. For ease of administration, it is preferred that the oral dosage form contains all the three drugs. The oral dosage forms may be in the form of tablets, troches, lozenges, aqueous, solid or semi-solid solutions or mixtures, or oily suspensions or solutions, dispersible powders or granules, emulsions, multiparticulate formulations, syrups, elixirs, and the like.

In other embodiments, a pharmaceutical composition containing the NMDA antagonist, anticonvulsant and/or a tricyclic anti-depressant and tramadol or its analog can be administered in dosage form as a topical preparation, a solid state and or depot type transdermal delivery device(s), a suppository, a buccal tablet, or an inhalation formulation such as a controlled release particle formulation or spray, mist or other topical vehicle, intended to be inhaled or instilled into the sinuses. Similarly, in other embodiments, a pharmaceutical composition containing the NMDA antagonist, anticonvulsant and/or a tricyclic anti-depressant, capsaicin or an ester of capsaicin and tramadol or its analog can be administered in dosage form as a topical preparation, a solid state and or depot type transdermal delivery device(s), a suppository, a buccal tablet, or an inhalation formulation such as a controlled release particle formulation or spray, mist or other topical vehicle, intended to be inhaled or instilled into the sinuses.

The pharmaceutical compositions containing the NMDA antagonist, anticonvulsant and/or a tricyclic anti-depressant and/or tramadol or its analog set forth herein may alternatively be in the form of microparticles such as microcapsules, microspheres and the like, which may be injected or implanted into a human patient, or other implantable dosage forms known to those skilled in the art of pharmaceutical formulation. Similarly, the pharmaceutical compositions containing the NMDA antagonist, capsaicin or an ester of capsaicin, anticonvulsant and/or a tricyclic anti-depressant and/or tramadol or its analog set forth herein may alternatively be in the form of microparticles such as microcapsules, microspheres and the like, which may be injected or implanted into a human patient, or other implantable dosage forms known to those skilled in the art of pharmaceutical formulation. For ease of administration, it is preferred that such dosage forms contain each drug.

Similarly, pharmaceutical compositions essentially free of a NSAID or acetaminophen and comprising a combination of a NMDA antagonist, an anticonvulsant and/or a tricyclic anti-depressant and a tramadol or its analog can be prepared in solid oral dosage forms or other dosage forms as described above. Accordingly, the pharmaceutical compositions can be administered orally, by means of an implant, parenterally, sub-dermally, sublingually, rectally, topically, or via inhalation.

Another embodiment of the invention is directed to a method of alleviating pain without the use of a narcotic analgesic. The method comprises administering to a patient a pharmaceutical composition comprising a NMDA antagonist, an anticonvulsant and/or a tricyclic anti-depressant and tramadol or its analog, or comprising a pharmaceutical composition essentially free of a NSAID or acetaminophen and comprising a combination of a NMDA antagonist, an anticonvulsant and/or a tricyclic anti-depressant and tramadol or its analog. In accordance with this embodiment, the active agents can be administered either together or separately, and the patient is not administered a narcotic analgesic.

Yet another embodiment of the invention is directed to a method of alleviating pain without the use of a narcotic analgesic. The method comprises administering to a patient a pharmaceutical composition comprising a NMDA antagonist, an anticonvulsant and/or a tricyclic anti-depressant, capsaicin or an ester of capsaicin and tramadol or its analog, or comprising a pharmaceutical composition essentially free of a NSAID or acetaminophen and comprising a combination of a NMDA antagonist, an anticonvulsant and/or a tricyclic anti-depressant, capsaicin or an ester of capsaicin and tramadol or its analog. In accordance with this embodiment, the active agents can be administered either together or separately, and the patient is not administered a narcotic analgesic.

### BRIEF DESCRIPTION OF THE DRAWINGS

The present disclosure will be provided with reference to the following drawings, in which like numerals refer to like elements, and in which:

The following drawings form part of the present specification and are included to further demonstrate certain aspects of the present invention. The invention may be better understood by reference to one or more of these drawings in combination with the detailed description of specific embodiments presented herein.

FIG. 1 provides the chemical structures of certain compounds which can be used in practicing the present invention.

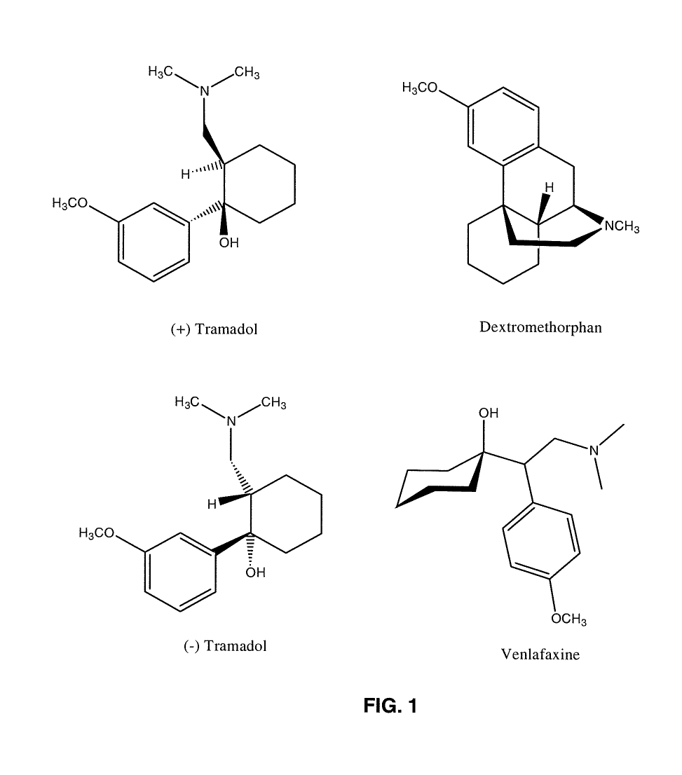

FIG. 2 provides the chemical structures of certain gabapentin analogs.

FIG. 3 provides chemical structures of certain capsaicin analogs and their esters.

FIG. 4 provides chemical structures of certain cyclic anti-depressant.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by the appended claims.

### DETAILED DESCRIPTION

### Definition of Terms

It should be understood that for purposes of the present invention, the following terms have the following meanings:

The term “effective analgesia” is defined for purposes of the present invention as a satisfactory reduction in or elimination of pain, along with the production of a tolerable level of side effects, as determined by the human patient.

The term “effective pain management” is defined for the purposes of the present invention as the objective evaluation or opinion of a human patient's response (pain experienced versus side effects) to analgesic treatment by a physician as well as subjective evaluation of therapeutic treatment by the patient undergoing such treatment. The skilled artisan will understand that effective analgesia will vary widely according to many factors, including individual patient variables.

The term “tramadol or its analog” is defined for purposes of the present invention as the drug in its base form, or a pharmaceutically acceptable salt or complex thereof. Even though it is known that the pure enantiomers of tramadol have a differing pharmaceutical profiles and effects when compared to the racemate as discussed in the background of the invention, it should be understood for the purpose of the invention, both the optical isomers and the recemic mixtures of tramadol will be referred simply as “tramadol or its analog”.

The term “NMDA antagonist” as used herein is intended to encompass compounds that deactivate the NMDA receptor. The NMDA receptor is a ligand gated ion channel that allows for the transfer of electrical signals between neurons in the brain and in the spinal column. For electrical signals to pass, the NMDA receptor must be open. To remain open, an NMDA receptor must bind to glutamate and to glycine. An NMDA receptor that is bound to glycine and glutamate and has an open ion channel is called “activated”. NMDA antagonists fall into four categories: Competitive antagonists, which bind to and block the binding site of the neurotransmitter glutamate; glycine antagonists, which bind to and block the glycine site; noncompetitive antagonists, which inhibit NMDARs by binding to allosteric sites; and uncompetitive antagonists, which block the ion channel by binding to a site within it. Examples of NMDA receptor antagonists include, but not limited to, dextromethorphan, magnesium, dextrorphan, ketamine, amantadine, memantine, eliprodil, ifenprodil, phencyclidine, MK-801, dizocilpine, CCPene, flupirtine, or derivatives or salts thereof. Even though, magnesium exerts various physiological effects, for the purpose of teaching the present invention, it will be simply be referred as NMDA receptor antagonist.

The term “dextromethorphan” is defined for purposes of the present invention as the drug in its base form, or a pharmaceutically acceptable salt or complex thereof.

The term “magnesium” is defined for purposes of the present invention as the pharmaceutically acceptable salt of magnesium which include, but not limited to, magnesium chloride, magnesium sulfate, magnesium gluconate, magnesium citrate, magnesium aspartate, magnesium lactate, magnesium levulinate, magnesium pidolate, magnesium orotate, magnesium oxide and magnesium malate.

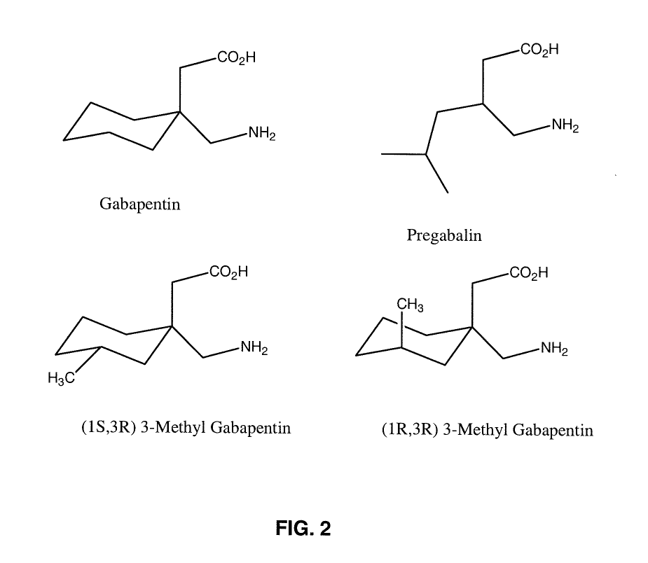

The term “anticonvulsant” as used herein is intended to encompass compounds which possess anti-epileptic activity and some of them bind to the family of proteins called α2δ. Examples of such compound include, but not limited to, sodium channel blockers such as carbamazepine, phenyloin, oxcarbazepine, lamotrigine and zonisamide, benzodiazepine analogs, valproate, glutamate blockers such as felbamate and topiramate, levetiracetam, gabapentin, derivatives or analogs of gabapentin or any compounded mixture thereof (see FIG. 2). Examples of analog of gabapentin include, but not limited to, pregabalin, 3-methyl-gabapentin, [(1R,5R,6S)-6-(Aminomethyl)bicyclo[-3.2.0]hept-6-yl]acetic acid, 3-(1-Aminomethyl-cyclohexylmethyl)-4H-[1,2,4]-oxadiazol-5-one, C-[1-(1H-Tetrazol-5-ylmethyl)-cycloheptyl]-methylamine, (3S,4S)-(1-Aminomethyl-3,4-dimethyl-cyclopentyl)-acetic acid, (1α,3α,5α)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid and (3S,5R)-3-Amino-5-methyl-octanoic acid, or a pharmaceutically acceptable salt thereof.

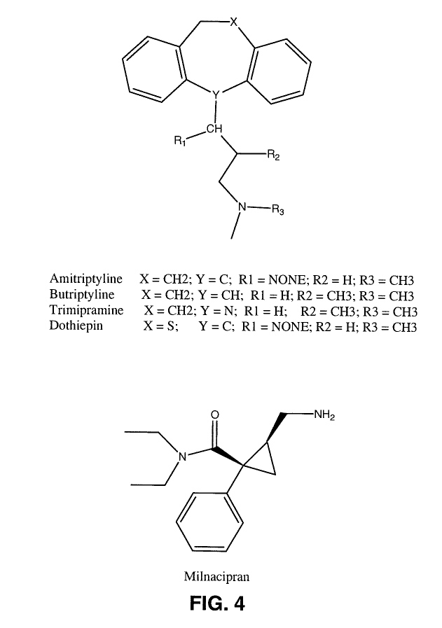

The term “tricyclic anti-depressant” (abbreviation TCA) as used herein is intended to encompass a class of anti-depressant drugs and these drugs are named after their molecular structures, which contain three rings of atoms (See FIG. 4). Prominent among the tricyclic anti-depressants are the linear tricyclics, e.g., imipramine, desipramine, amitriptyline, nortriptyline, protriptyline, doxepin, ketipramine, mianserin, dothiepin, amoxapine, dibenzepin, melitracen, maprotiline, flupentixol, azaphen, tianeptine and related compounds showing similar activity. Angular tricyclics include indriline, clodazone, nomifensin, and related compounds. A variety of other structurally diverse anti-depressants, e.g., iprindole, wellbatrin, nialamide, milnacipran, phenelzine and tranylcypromine have been shown to produce similar activities (Sellinger et al, 1979; Pandey et al, 1979; and Moret et al, 1985). They are functionally equivalent to the tricyclic anti-depressants and are therefore included within the scope of the invention. Thus, the term tricyclic anti-depressant is intended by the present inventor to embrace the broad class of anti-depressants described above together with related compounds sharing the common property that they all possess anti-depressant activity.

The term “an anticonvulsant and/or a tricyclic anti-depressant” as used herein is intended to encompass either a combination of an anticonvulsant and a tricyclic anti-depressant or an anticonvulsant alone or a tricyclic anti-depressant alone.

The term “chronic pain” means pain associated with an idiopathic or undiagnosed or an undiagnosible disease, disorder or condition, or pain associated with any one of: myofascial pain syndrome, trigger points, tender points, thorasic outlet syndrome, complex regional pain syndrome, reflex sympathetic dystrophy (RSD), sympathetically maintained pain (SMP), diabetic neuropathy syndrome (DNS); chronic pain associated with fibromyalgia syndrome (FMS), multiple sclerosis (MS); chronic pain associated with traumatic injury to the peripheral nervous system; chronic pain resulting from herpes zoster (also known as shingles, or post-herpetic neuropathy) or similar infections that attack and damage nerve fibers or endings; post-operative pain, which arises after surgery and then lingers far beyond a normal convalescent period; pain associated with nerve and root damage, such as pain associated with peripheral nerve disorders, including, nerve entrapment and brachial plexus avulsions, amputation, peripheral neuropathies, tic douloureux, atypical facial pain, nerve root damage, and arachnoiditis syndrome, in which an amputee suffers from feelings of pain or discomfort that seems to originate in the missing limb (“phantom limb” pain); pain associated with carcinoma, often referred to as cancer pain; neuropathic pain associated with chemotherapy treatment; central nervous system pain, including pain due to spinal cord or brain stem damage; low back pain; sciatica; headache, including migraine, chronic tension headache, cluster headache, temporomandibular disorder (TMJ) pain and maxillary sinus pain; complex regional pain syndromes, including reflex sympathetic dystrophy and causalgia, or from burn injury; the chronic pain associated with hyperesthesia, allodynia, hyperalgesia, deafferentation pain, sympathetically maintained pain, non-nociceptive chronic pain.

The term “pain relieving” is generally defined herein to include the expressions “pain-suppressing”, “pain-reducing”, and “pain-inhibiting” as the invention is applicable to the alleviation of existing pain, as well as the suppression or inhibition of pain which would otherwise ensue from the imminent pain-causing event.

The term “sustained or controlled release” is defined for purposes of the present invention as the release of the drug (tramadol or its analog) from the transdermal formulation at such a rate that blood (plasma) concentrations (levels) of the drugs are maintained within the therapeutic range that is above the minimum effective analgesic concentration or “MEAC”, but below toxic levels over a period of time of several hours to several days.

The term “steady state” means that the blood plasma time/concentration curve for a given drug level has been substantially stable within a set range from dose to dose.

The term “minimum effective analgesic concentration” or “MEAC” is defined for purposes of this invention as the minimum effective therapeutic blood plasma level of the drug at which at least some pain relief is achieved in a given patient. It will be well understood by those skilled in the medical art that pain measurement is highly subjective and great individual variations may occur among patients.

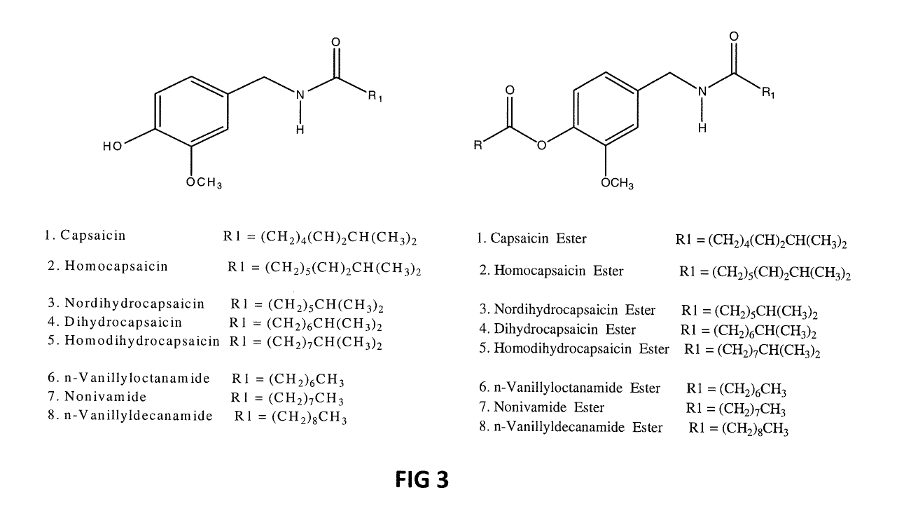

The term “capsaicin” or “capsaicins” as used herein is intended to encompass not only the compound capsaicin, but also homocapsaicin, nordihydrocapsaicin, dihydrocapsaicin, homodihydrocapsaicin or any compounded mixture thereof (see FIG. 3).

It must be noted that, as used in this specification, the singular forms “a,” “an” and “the” include plural referents unless the context clearly dictates otherwise. Thus, for example, reference to “a pharmacologically active agent” includes a combination of two or more pharmacologically active agents, and the like.

As used herein in the claim(s), when used in conjunction with the word “comprising”, the words “a” or “an” may mean one or more than one. As used herein “another” may mean at least a second or more.

### DESCRIPTION OF THE APPLICATIONS OF THE INVENTION

The pharmacological management of acute postoperative pain and chronic pain syndromes has been traditionally based on various regimens of opiates and their congeners or NSAIDs. All opiates have side effects, of which the most dangerous are respiratory and cardiovascular depression associated with excessive sedation. NSAIDs may also induce side effects such as exacerbation of bleeding tendencies and the impairment renal function. The search for alternative pain control strategies has focused on the N-methyl-D-aspartate (NMDA) receptors and their antagonists which were recently shown to alleviate somatic and neuropathic pain sensation in both animal and human models. The clinical utility of these agents stems from the high affinity binding of the drugs to NMDA receptors resulting in blockade of the NMDA receptors located at the junction where pain is generated by peripheral nociceptive stimuli and is thence conveyed to central receptors via A * and C sensory fibers. From a clinical standpoint, the amounts of conventional pain killers that are needed for effective pain control would be much smaller. One of these compounds is dextromethorphan (DM), a low affinity, non-competitive NMDA receptor antagonist that has a long history of clinical safety as a cough suppressant.

Considerable evidence has accumulated over the past few years on the role of excitatory amino acids (EAA), such as glutamate and aspartate, in modulating the sensation of pain via the ascending pathways along the spinal cord and central nervous system. The stimulation of NMDA receptors located in the dorsal horn of the spinal cord, the area responsible for relaying, modulating and transmitting pain, by intraspinal deposition of glutamate in experimental rat and monkey models generated an increased response to noxious stimuli and lowered the threshold of pain. This response was successfully abolished by administration of NMDA antagonists, such as phencyclidine, suggesting that the pain can be attenuated by blocking the activity of these receptors.

Investigations of chronic pain syndromes revealed that the same mechanisms are involved in the initiation and the perpetuation of secondary pain in mouse and rat models. In terms of neurophysiology, following acute tissue injury, transduction is accomplished by action potentials being generated at the nerve endings and transmitted along the A * and C fibres to the synapses of the dorsal part of the spinal cord where they induce the release of various peptides, including EAA. The EAA activate the NMDA receptors that are located within the synapses, thus stimulating the synaptic neurons to transmit sensations of pain. This state of hyperexcitability, or “wind up” amplifies the magnitude and duration of neurogenic responses to any existing volley of nociceptive activity. Once initiated, this state of hyperex-citability can exist even after the peripheral input has ceased Dickenson 1995). This phenomenon is currently thought to be responsible for various clinical pain syndromes such as allodynia, an intense sensation of pain following a relatively minor stimulus that would not ordinarily induce pain sensation or hyperpathia, a sensation of pain that persists long after the initial nociceptive stimulus has subsided.

The role of NMDA in the “wind up” phenomenon of pain perception was clarified in animals by intraspinal administration of NMDA-receptor antagonists. In one human study, i.v. ketamine reduced the magnitude of both primary (immediate) and secondary hyperalgesia and the pain evoked by prolonged heat stimulation in a dose-dependent manner. DM acts in a similar manner: Klepstad et al, published a case report of a patient who had undergone four years of satisfactory ketamine treatment for postherpetic neuralgia. Experimental substitution of the ketamine by DM 125 mg in four divided doses for seven days was found to be as efficient. Here it is important to note that the NMDA receptors are widespread throughout the central nervous system, and as such, are associated with highly diverse neurophysiological functions as far removed from the modulation of pain as learning and memory processing.

It is therefore not surprising that their antagonists can interfere with its physiological activity, leading to sedation, motor dysfunction or altered behavior. Antagonism of the potentially deleterious effects of an excessive release of EAA, such as that which occurs in patients with focal brain ischemia (an example of the diversity of NMDA activity) can lead to episodes of agitation, hallucinations, somnolence, nausea, vomiting and nystagmus. This is why so few NMDA receptor antagonists have been tested in humans despite their effectiveness in pain management, and despite the extensive animal data that point to their promising beneficial effect.

To date DM, ketamine and amantadine are the only drugs with NMDA receptor antagonistic properties that are FDA approved drugs for clinical use. However, due to the high affinity of ketamine to its receptors and its related dysphoric effects, together with the need to administer it intravenously, research in pain control has turned its focus to DM as the preferred NMDA antagonist for clinical use.

Dextromethorphan and levorphanol were originally synthesized as pharmacological alternatives to morphine more than 40 years ago. DM is the D isomer of the codeine analogue, levorphanol but, in contrast to its L isomer, it has no effect on the opiate receptors. From the beginning, its clinical use was mainly that of an antitussive in syrup preparations, at adult doses of 10 to 30 mg three to six times daily. The specific central sites upon which DM exerts its antitussive effect are still uncertain, but they are distinct from those of opiates, insofar as the effect is not suppressed by naloxone (Karlsson et al, 1988). Also, unlike opiates, DM has an established safety record, i.e., the therapeutic cough suppressant dose (1 mg·kg-1·dy-1) has no major opiate like respiratory or hemodynamic side effects, neither does it induce histamine release complications. The binding of the antagonists to the NMDA receptors results in modifying the receptor-gated Ca2+ current. Changes in the Ca2+ current normally lead to NMDA induced neuronal firing which, if it persists, is followed by a heightening of the intensity of the primary nociceptive stimulus, i.e., “wind up” phenomenon, and the triggering of secondary sensory pain. In contrast to the other NMDA receptor antagonists, DM has widespread binding sites in the central nervous system that are distinct from those of opiates and other neurotransmitters, so that its activity is not limited to the NMDA receptors alone, as was shown in pigs and rats. Besides the ability of DM to reduce intracellular Ca2+ influx through the NMDA receptor-gated channels, DM also regulates voltage-gated Ca2+ channels that are normally activated by high concentrations of extracellular K+. One of the physiological consequences of these multi-channel regulation capabilities is the attenuation by DM of NMDA mediated neuronal firing in the brain that is normally transformed into seizures, as was shown experimentally in rats and in neuronal cell cultures as well as in humans.

The neuropharmacological cascade of events that provokes the reduced intracellular accumulation of Ca2+ to cause changes in the activity of NMDA receptors remains to be elucidated. In humans as in animals, DM was also capable of ameliorating discomfort associated with excitotoxicity-related neurological disorders, such as intractable seizures and Parkinson's disease when administered at doses of 30 or 60 mg q.i.d., 45 to 180 mg p.o. or 120 mg p.o. for periods of three weeks to three months. No serious untoward neurological effects were detected in these and in another study where eight healthy human volunteers in whom motor cortex excitability, as indicated by motor-evoked potentials, was reduced after a single oral high (150 mg) dose. In addition, motor cortex excitability and levodopa-induced dyskinesis were reduced by DM at a dose of 100 mg in a double-blind placebo-control study in patients with Parkinson's disease, with only negligible side effects.

### Elaboration of the Properties of the Preferred Active Ingredients

Dextromethorphan is rapidly metabolized in the liver where it is transformed to dextrorphan, its active and more potent derivative as a NMDA antagonist. It was suggested that the side effects documented in clinical studies and attributed to the oral administration of DM might be mediated by this metabolite acting at the phencyclidine receptorial site rather than DM itself.

Satisfactory pain control achieved with the least amount of opiates has always been an important goal in view of both the psychological and somatic dependence these drugs may induce and the often intolerable side effects that may follow their extensive use. The searchers for techniques of pain control that will afford full orientation, coordination and collaboration, and normal respiration as well as stable hemodynamics view these factors as important cornerstones in postoperative planning of pain control. This applies equally to patients who had undergone either general or regional anesthesia and to inpatients as well as outpatients. Moreover, in view of the contention that persistent NMDA receptor activation can evoke central hyperexcitability that can lead to secondary pain, proper pain control should both modulate primary pain sensation and preempt an analgesic state that would prevent acute pain from progressing into chronic pain. This concept of preemptive analgesia (i.e., reducing pain sensation in advance) is feasible via NMDA modulation, as had been demonstrated by the administration of opiates and ketamine to patients before surgery. Importantly, this neuropharmacological receptor conditioning is also beneficial for reducing the need for additional doses of opiates post-operatively. In addition, while the neurovegetative stimulation and adrenergic overproduction that accompany the continuous neurally transmitted acute and, to a greater extent, secondary pain are clearly detrimental to all patients, they may be particularly harmful for cardiac patients. In this regard, the preemptive approach is an especially promising and beneficial one. The use of DM may, therefore, become an established component in protocols of treating pain and of alleviating the accompanying neurovegetative phenomena. Finally, the bioavailability of DM administered orally makes it much more convenient than the other anti-NMDA drugs, all of which are administered by injection, such as ketamine. As a potential morphine sparing agent for pain, the use of DM was shown to be efficient and well tolerated.

It is noteworthy that NMDA receptor antagonists, including DM, are not in themselves anti-nociceptive but rather they inhibit central sensitization and, thus, the perception of primary and secondary pain. The preemptive use of these antagonists, while blunting the development of a central sensitization of a nociceptive stimulus, still requires the use of an analgesic for complete abolition of pain perception.

Additional substances that block a major intracellular consequence of NMDA receptor activation and as such are useful in the practice of the invention include inhibitors of calmodulin such as the phenothiazines, in particular, chlorpromazine, chlorpromazine sulfoxide, prochlorperazine dimaleate, perphenazine, trifluoperazine, fluphenazine, fluphenazine enanthate, fluphenazine decanoate, thioridazine, mesoridazine besylate, piperacetazine, acetophenazine dimaleate, carphenazine dimaleate, butaperazine dimaleate and phenothiazine sulfoxide; naphthalenesulfonamides such as N-(6-aminohexyl)-5-chloro-1-naphthalenesulfonamide, N-(6-aminohexyl)-5-chloro-2-naphthalenesulfonamide and N-(6-aminohexyl)-5-bromo-2-naphthalenesulfonamide; 4-substituted-4H,6H-pyrrolo[1,2-a][4,1]benzoxazepines such as 1,3-dihydro-1-{1-[(4-methyl-4H,6H-pyrrolo[1,2-a][4,1]benzoxazepin-4-yl)methyl]-4-piperidinyl}-2H-benzimidazol-2-one; benzhydryls such as N-[2](diphenylmethylthioethyl)-2-(trifluoromethyl)-benzeneethanamine, N-[2-(bis(4-fluorophenyl)methylthio)-)ethyl]-2-(trifluoromethyl)benzene ethanamine and N-(bis(4-fluorophenyl)methylthio)ethyl]-3-(trifluoromethyl)benzene ethanamine; tricyclic antidepressant drugs such as imipramine, 2-chloroimipramine and amitriptyline; penfluridol; haloperidol; pimozide; clozapine; calmidazolin; and, mixtures and pharmaceutically acceptable salts of any of the foregoing.

(+/−)-Tramadol is a synthetic 4-phenyl-piperidine analogue of codeine. It is a central analgesic with a low affinity for opiate receptors. Its selectivity for μ receptors has recently been demonstrated, and the M1 metabolite of tramadol, produced by liver O-demethylation, shows a higher affinity for opiate receptors than the parent drug. The rate of production of this M1 derivative (O-demethyl tramadol), is influenced by a polymorphic isoenzyme of the debrisoquine-type, cytochrome P450 2D6 (CYP2D6). One mechanism relates to its weak affinity for □-opiate receptors (6,000-fold less than morphine, 100-fold less than d-propoxyphene, 10-fold less than codeine, and equivalent to dextromethorphan). Moreover, and in contrast to other opiates, the analgesic action of tramadol is only partially inhibited by the opiate antagonist naloxone, which suggests the existence of another mechanism of action. This was demonstrated by the discovery of a monoaminergic activity that inhibits noradrenaline (norepinephrine) and serotonin (5-hydroxytryptamine; 5-HT) reuptake, making a significant contribution to the analgesic action by blocking nociceptive impulses at the spinal level.

(+/−)-Tramadol is a racemic mixture of 2 enantiomers, each one displaying differing affinities for various receptors. (+/−)-tramadol is a selective agonist of □ receptors and preferentially inhibits serotonin reuptake, whereas (−)-tramadol mainly inhibits noradrenaline reuptake. The action of these 2 enantiomers is both complementary and synergistic and results in the analgesic effect of (+/−)-tramadol. After oral administration, tramadol demonstrates 68% bioavailability, with peak serum concentrations reached within 2 hours. The elimination kinetics can be described as 2-compartmental, with a half-life of 5.1 hours for tramadol and 9 hours for the M1 derivative after a single oral dose of 100 mg. This explains the approximately 2-fold accumulation of the parent drug and its M1 derivative that is observed during multiple dose treatment with tramadol. The recommended daily dose of tramadol is between 50 and 100 mg every 4 to 6 hours, with a maximum dose of 400 mg/day. The duration of the analgesic effect after a single oral dose of tramadol 100 mg is about 6 hours. Adverse effects, and nausea in particular, are dose dependent and therefore considerably more likely to appear if the loading dose is high. The reduction of this dose during the first days of treatment is an important factor in improving tolerability. Other adverse effects are generally similar to those of opiates, although they are usually less severe, and can include respiratory depression, dysphoria and constipation. Tramadol can be administered concomitantly with other analgesics, particularly those with peripheral action, while drugs that depress CNS function may enhance the sedative effect of tramadol. Tramadol has pharmacodynamic and pharmacokinetic properties that are highly unlikely to lead to dependence. This was confirmed by various controlled studies and postmarketing surveillance studies, which reported an extremely small number of patients developing tolerance or instances of tramadol abuse. Although it has proven to be a safe and effective agent for the control of pain, adverse effects can occur with its use. It has been reported the occurrence of seizure activity after the inadvertent administration of 4 mg/kg of tramadol to a child.

Venlafaxine is a novel SSRI chemically unrelated to other SSRIs but chemically similar to the tramadol. The chemical structures of venlafaxine and tramadol are similar, demonstrating the similarity between these two antidepressant and analgesic substances, respectively. It is designated (R/S)-1-[2-(dimethylamino)-1-(4-methoxyphenyl)ethyl]cyclohexanol or (±)-1-[a-[(dimethylamino)methyl]-p-methoxybenzyl]cyclohexanol and has the empirical formula of C17H27NO2. Venlafaxine hydrochloride is a white to off-white crystalline solid with a solubility of 572 mg/mL in water (adjusted to ionic strength of 0.2 M with sodium chloride. Its octanol:water (0.2M sodium chloride) partition coefficient is 0.43. Venlafaxine hydrochloride (Effexor) is formulated as capsule for oral administration. Capsules contain venlafaxine hydrochloride equivalent to 37.5 mg, 75 mg, or 150 mg venlafaxine.

The mechanism of the antidepressant action of venlafaxine in humans is believed to be the same as with other SSRIs, associated with its potentiation of neurotransmitter activity in the CNS as with other SSRIs: preclinical studies have shown that venlafaxine and its active metabolite, O-desmethylvenlafaxine (ODV), are potent inhibitors of neuronal serotonin and norepinephrine reuptake and weak inhibitors of dopamine reuptake. That venlafaxine is analgesia is seen in studies in animals that show that venlafaxine is effective in reversing chronic neuropathic pain secondary to thermal hyperalgesia, and additionally is effective in treating the hyperalgesia of neuropathic pain due to chronic sciatic nerve constriction injury in rats (Lang 1998). Venlafaxine-induced antinociception is significantly inhibited by naloxone, nor-BNI and naltrindole but not by β-FNA or naloxonazine, implying involvement of κ1- and δ-opioid mechanisms. When adrenergic and serotoninergic antagonists are used, yohimbine but not phentolamine or metergoline, decreased antinociception elicited by venlafaxine, implying a clear α2- and a minor α1-adrenergic mechanism of antinociception. Therefore, the antinociceptive effect of venlafaxine is mainly influenced by the κ- and δ-opioid receptor subtypes combined with the α2-adrenergic receptor. These results suggest a potential use of venlafaxine in the management of some pain syndromes. However, further research is needed in order to establish both the exact clinical indications and the effective doses of venlafaxine when prescribed for neuropathic pain (Schreiber 1999).

Gabapentin (GBP; Neurontin®) is an anticonvulsant that has found increased utility for the treatment of clinical neuropathic pain. Although originally developed for the treatment of spasticity and epilepsy, recent attention has focused on the utility of GBP for the treatment of neuropathic pain based on its efficacy and minimal side-effect profile in clinical trials (Rice and Maton, 2001). In rodent neuropathic pain models, Additionally, GBP inhibits spontaneous nociceptive behaviors and mechanical hyperalgesia produced by intraplantar formalin or surgical incision, respectively. The antinociceptive effects of GBP in models of neuropathic, inflammatory, and surgical pain appear to be selective for injury-induced hypersensitivity, since responses to acute noxious stimuli are unaffected.

Gabapentin is commercially supplied as Neurontin® Capsules, Neurontin® Tablets, and Neurontin® Oral Solution, as imprinted hard shell capsules containing 100 mg, 300 mg, and 400 mg of gabapentin, elliptical film-coated tablets containing 600 mg and 800 mg of gabapentin or an oral solution containing 250 mg/5 mL of gabapentin. Gabapentin bioavailability is not dose proportional; i.e., as dose is increased, bioavailability decreases. Bioavailability of gabapentin is approximately 60%, 47%, 34%, 33%, and 27% following 900, 1200, 2400, 3600, and 4800 mg/day given in 3 divided doses, respectively. Food has only a slight effect on the rate and extent of absorption of gabapentin (14% increase in AUC and Cmax). Less than 3% of gabapentin circulates bound to plasma protein. The apparent volume of distribution of gabapentin after 150 mg intravenous administration is 58±6 L (Mean±SD). In patients with epilepsy, steady-state predose (Cmin) concentrations of gabapentin in cerebrospinal fluid were approximately 20% of the corresponding plasma concentrations. Gabapentin is eliminated from the systemic circulation by renal excretion as unchanged drug. Gabapentin is not appreciably metabolized in humans. Gabapentin elimination half-life is 5 to 7 hours and is unaltered by dose or following multiple dosing. Gabapentin elimination rate constant, plasma clearance, and renal clearance are directly proportional to creatinine clearance. In elderly patients, and in patients with impaired renal function, gabapentin plasma clearance is reduced. Gabapentin can be removed from plasma by hemodialysis.

Currently gabapentin is indicated as adjunctive therapy in the treatment of partial seizures with and without secondary generalization in patients over 12 years of age with epilepsy. Gabapentin is also indicated as adjunctive therapy in the treatment of partial seizures in pediatric patients age 3¾ 12 years.

In vitro studies were conducted to investigate the potential of gabapentin to inhibit the major cytochrome P450 enzymes (CYP1A2, CYP2A6, CYP2C9, CYP2C19, CYP2D6, CYP2E1, and CYP3A4) that mediate drug and xenobiotic metabolism using isoform selective marker substrates and human liver microsomal preparations. Only at the highest concentration tested (171 μg/mL; 1 mM) was a slight degree of inhibition (14%-30%) of isoform CYP2Aδ observed. No inhibition of any of the other isoforms tested was observed at gabapentin concentrations up to 171 μg/mL (approximately 15 times the Cmax at 3600 mg/day).

Gabapentin is not appreciably metabolized nor does it interfere with the metabolism of commonly coadministered antiepileptic drugs. Gabapentin is given orally with or without food. In adults with postherpetic neuralgia, gabapentin therapy may be initiated as a single 300-mg dose on Day 1, 600 mg/day on Day 2 (divided BID), and 900 mg/day on Day 3 (divided TID). The dose can subsequently be titrated up as needed for pain relief to a daily dose of 1800 mg (divided TID). In clinical studies, efficacy was demonstrated over a range of doses from 1800 mg/day to 3600 mg/day with comparable effects across the dose range. Additional benefit of using doses greater than 1800 mg/day was not demonstrated. For patients >12 years of age: The effective dose of Neurontin is 900 to 1800 mg/day and given in divided doses (three times a day) using 300 or 400 mg capsules, or 600 or 800 mg tablets. The starting dose is 300 mg three times a day. If necessary, the dose may be increased using 300 or 400 mg capsules, or 600 or 800 mg tablets three times a day up to 1800 mg/day. Dosages up to 2400 mg/day have been well tolerated in long-term clinical studies. Doses of 3600 mg/day have also been administered to a small number of patients for a relatively short duration, and have been well tolerated. The maximum time between doses in the TID schedule should not exceed 12 hours.

The most commonly observed adverse events associated with the use of gabapentin in adults, not seen at an equivalent frequency among placebo-treated patients, were dizziness, somnolence, and peripheral edema. The most commonly observed adverse events associated with the use of gabapentin in combination with other antiepileptic drugs in patients >12 years of age, not seen at an equivalent frequency among placebo-treated patients, were somnolence, dizziness, ataxia, fatigue, and nystagmus. The most commonly observed adverse events reported with the use of gabapentin in combination with other antiepileptic drugs in pediatric patients 3 to 12 years of age, not seen at an equal frequency among placebo-treated patients, were viral infection, fever, nausea and/or vomiting, somnolence, and hostility.

Pregabalin, an analog of gabapentin, is sold commercially as pregabalin capsules and is administered orally and are supplied as imprinted hard-shell capsules containing 25, 50, 75, 100, 150, 200, 225, and 300 mg of pregabalin, along with lactose monohydrate, cornstarch, and talc as inactive ingredients. The capsule shells contain gelatin and titanium dioxide. In addition, the orange capsule shells contain red iron oxide and the white capsule shells contain sodium lauryl sulfate and colloidal silicon dioxide. Colloidal silicon dioxide is a manufacturing aid that may or may not be present in the capsule shells. The imprinting ink contains shellac, black iron oxide, propylene glycol, and potassium hydroxide.

Treatment with pregabalin 100 and 200 mg three times a day statistically significantly improved the endpoint mean pain score and increased the proportion of patients with at least a 50% reduction in pain score from baseline. There was no evidence of a greater effect on pain scores of the 200 mg three times a day dose than the 100 mg three times a day dose, but there was evidence of dose dependent adverse reactions. A 13-week study compared pregabalin 75, 150, and 300 mg twice daily with placebo. Patients with creatinine clearance (CLcr) between 30 to 60 mL/min were randomized to 75 mg, 150 mg, or placebo twice daily. Patients with creatinine clearance greater than 60 mL/min were randomized to 75 mg, 150 mg, 300 mg or placebo twice daily. In patients with creatinine clearance greater than 60 mL/min treatment with all doses of pregabalin statistically significantly improved the endpoint mean pain score and increased the proportion of patients with at least a 50% reduction in pain score from baseline. Despite differences in dosing based on renal function, patients with creatinine clearance between 30 to 60 mL/min tolerated pregabalin less well than patients with creatinine clearance greater than 60 mL/min as evidenced by higher rates of discontinuation due to adverse reactions. Some patients experienced a decrease in pain as early as Week 1, which persisted throughout the study.

A 8-week study compared pregabalin 100 or 200 mg three times a day with placebo, with doses assigned based on creatinine clearance. Patients with creatinine clearance between 30 to 60 mL/min were treated with 100 mg three times a day, and patients with creatinine clearance greater than 60 mL/min were treated with 200 mg three times daily. Treatment with pregabalin statistically significantly improved the endpoint mean pain score and increased the proportion of patients with at least a 50% reduction in pain score from baseline. Some patients experienced a decrease in pain as early as Week 1, which persisted throughout the study.

A 8-week study compared pregabalin 50 or 100 mg three times a day with placebo with doses assigned regardless of creatinine clearance. Treatment with pregabalin 50 and 100 mg three times a day statistically significantly improved the endpoint mean pain score and increased the proportion of patients with at least a 50% reduction in pain score from baseline. Patients with creatinine clearance between 30 to 60 mL/min tolerated pregabalin less well than patients with creatinine clearance greater than 60 mL/min as evidenced by markedly higher rates of discontinuation due to adverse reactions. Some patients experienced a decrease in pain as early as Week 1, which persisted throughout the study.

A 14-week study compared pregabalin total daily doses of 300 mg, 450 mg and 600 mg with placebo. Patients were enrolled with a minimum mean baseline pain score of greater than or equal to 4 on an 11-point numeric pain rating scale and a score of greater than or equal to 40 mm on the 100 mm pain visual analog scale (VAS). The baseline mean pain score in this trial was 6.7. Responders to placebo in an initial one-week run-in phase were not randomized into subsequent phases of the study. A total of 64% of patients randomized to pregabalin completed the study. There was no evidence of a greater effect on pain scores of the 600 mg daily dose than the 450 mg daily dose, but there was evidence of dose-dependent adverse reactions.

The maximum recommended dose of pregabalin for neuropathic pain associated with diabetic peripheral neuropathy is 100 mg three times a day (300 mg/day) in patients with creatinine clearance of at least 60 mL/min. Dosing should begin at 50 mg three times a day (150 mg/day) and may be increased to 300 mg/day within 1 week based on efficacy and tolerability. Because pregabalin is eliminated primarily by renal excretion, the dose should be adjusted for patients with reduced renal function. Although pregabalin was also studied at 600 mg/day, there is no evidence that this dose confers additional significant benefit and this dose was less well tolerated. In view of the dose-dependent adverse reactions, treatment with doses above 300 mg/day is not recommended.

The recommended dose of pregabalin for fibromyalgia is 300 to 450 mg/day. Dosing should begin at 75 mg two times a day (150 mg/day) and may be increased to 150 mg two times a day (300 mg/day) within 1 week based on efficacy and tolerability. Patients who do not experience sufficient benefit with 300 mg/day may be further increased to 225 mg two times a day (450 mg/day). Although pregabalin was also studied at 600 mg/day, there is no evidence that this dose confers additional benefit and this dose was less well tolerated. In view of the dose-dependent adverse reactions, treatment with doses above 450 mg/day is not recommended. Because pregabalin is eliminated primarily by renal excretion, the dose should be adjusted for patients with reduced renal function (creatinine clearance less than 60 mL/min).

In clinical trials in patients with neuropathic pain associated with diabetic peripheral neuropathy, 9% of patients treated with pregabalin and 4% of patients treated with placebo discontinued prematurely due to adverse reactions. In the pregabalin treatment group, the most common reasons for discontinuation due to adverse reactions were dizziness (3%) and somnolence (2%). In comparison, <1% of placebo patients withdrew due to dizziness and somnolence. Other reasons for discontinuation from the trials, occurring with greater frequency in the pregabalin group than in the placebo group, were asthenia, confusion, and peripheral edema. Each of these events led to withdrawal in approximately 1% of patients.

In clinical trials of patients with fibromyalgia, 19% of patients treated with pregabalin (150-600 mg/day) and 10% of patients treated with placebo discontinued prematurely due to adverse reactions. In the pregabalin treatment group, the most common reasons for discontinuation due to adverse reactions were dizziness (6%) and somnolence (3%). In comparison, <1% of placebo-treated patients withdrew due to dizziness and somnolence. Other reasons for discontinuation from the trials, occurring with greater frequency in the pregabalin treatment group than in the placebo treatment group, were fatigue, headache, balance disorder, and weight increased. Each of these adverse reactions led to withdrawal in approximately 1% of patients.

Amitriptyline, nortriptyline, and desipramine have been established as analgesics independent of their antidepressant effects. Although their mechanism of analgesic action has not been clearly defined, tricyclic antidepressants are thought to have an inhibitory effect on nociceptive pathways by blocking the reuptake of serotonin and norepinephrine. Originally, the major mechanism of the analgesic effect of tricyclic antidepressants was believed to be related to serotonin reuptake inhibition. Animal models of peripheral neuropathic pain have shown that tricyclic antidepressants act as sodium channel blockers, similar to local anesthetic and antiarrhythmic agents.

Amitriptyline drug is effective in the treatment of postherpetic neuralgia, diabetic neuropathy, and other neuropathic pain syndromes. Oral amitriptyline achieves a good or moderate response in about two-thirds of patients with postherpetic neuralgia and three-quarters of patients with painful diabetic neuropathy; such neurogenic pain syndromes are often unresponsive to narcotic analgesics. Whether analgesic effects of amitriptyline are linked to its mood-altering activity and/or are attributable to a discrete pharmacological action is unknown. Above the therapeutic plasma concentration of 0.3 to 0.8 μM, the tricyclic antidepressants have significant effects on the cardiovascular system, including direct depression of the myocardium and evidence of prolonged conduction times; with an overdose of >3 μM, these effects may be life-threatening (Amsterdam et al., 1980). The known physiological targets of tricyclic antidepressants in the central nervous system are the 5-HT2 serotonin receptors and the α1-adrenergic receptors.

Capsaicin is a natural constituent in pungent red chili peppers. Depending on the concentration used and the mode of application, capsaicin can selectively activate, desensitize, or exert a neurotoxic effect on small diameter sensory afferent nerves while leaving larger diameter afferents unaffected. Sensory neuron activation occurs due to interaction with a ligand-gated nonselective cation channel termed the vanilloid receptor (VR-1), and receptor occupancy triggers Na⁺ and Ca²⁺ ion influx, action potential firing, and the consequent burning sensation associated with spicy food or capsaicin-induced pain. VR1 receptors are present on both C and Aδ fibers, and can be activated by capsaicin and its analogs, heat, acidification, and lipid metabolites. Desensitization occurs with repeated administration of capsaicin, is a receptor-mediated process, and involves Ca²⁺- and calmodulin-dependent processes and phosphorylation of the cation channel.

Capsaicin excites a subset of primary sensory neurons with somata in dorsal root ganglion (DRG) or trigeminal ganglion by the activation of vanilloid-sensitive receptor TRPV1. Vanilloid-sensitive neurons are heterogeneous morphologically, neurochemically, and functionally, and they encompass several subclasses of DRG neurons. Because, sensitivity to vanilloids is the only known trait that all of these neurons seem to share, they are best described as vanilloid-sensitive neurons.

Among irritant compounds acting on primary sensory neurons, capsaicin and related vanilloids are unique in that the initial stimulation by vanilloids is followed by a lasting refractory state, traditionally termed desensitization. Unlike capsaicin, the palmitate ester of capsaicin does not stimulate the primary sensory neurons and does not produce the burning sensation at the application site.

TRPV1 receptor is a nonselective cation channel with high Ca2+ permeability and is the molecular target of capsaicin. Capsaicin also causes a synaptic transmission block in the spinal cord dorsal horn. The voltage-activated Ca2+ channels (VACCs) play a critical role in signal transduction, synaptic neurotransmitter release, and nociceptive transmission. VACCs also are an important molecular target of many analgesic drugs such as opioids. Interestingly, capsaicin causes a profound inhibition of VACC currents in dorsal root ganglion (DRG) neurons. Protein kinases and phosphatases are key enzymes in signal transduction pathways for a wide range of cellular processes. The enzymatic addition or removal of phosphate esters on serine and threonine hydroxyls alters the activity of many proteins that are essential to the characteristic structure and function of neurons. An important mechanism regulating VACC function is through phosphorylation by protein kinases and phosphatases. It has been show that a Ca2+-dependent serine/threonine phosphatase, calcineurin (protein phosphatase 2B), is critically involved in down-regulation of high voltage-activated Ca2+ channels (HVACCs) by capsaicin in native DRG neurons. Furthermore, the basal intracellular Ca2+ level and endogenous protein phosphatases tonically modulate the HVACC current in DRG neurons. These findings are important to the understanding of the functional interaction between TRPV1 and HVACCs in primary nociceptors.

In summary, there is substantial new evidence that calcineurin, constitutively expressed in the cytoplasm of DRG neurons, is a key feedback regulator of intracellular Ca2+ and plays an important role in down-regulation of HVACCs by TRPV1 stimulation. Increased calcineurin activity produced by TRPV1 activation could limit Ca²⁺ influx through HVACCs in the plasma membrane by down-regulation of HVACCs through dephosphorylating the HVACC or a closely associated cytoskeletal protein. This new information is important for the understanding of the molecular mechanism of the analgesic action and diminished spinal synaptic transmission produced by capsaicin. This finding also highlights the pivotal role of intracellular Ca²⁺ level and calcineurin in negative modulation of HVACCs in primary sensory neurons.

As discussed before, the vanilloid-1 (TRPV1) receptor is involved in peripheral and spinal nociceptive processing and is a therapeutic target for pain. It has been recently shown that TRPV1 in the ventrolateral periaqueductal gray (VL-PAG) tonically contributes to brain stem descending antinociception by stimulating glutamate release into the rostral ventromedial medulla and OFF neuron activity. Both opioid and vanilloid systems integrate and transduce pain sensation in these pathways and it has been found that the TRPV1 agonist, capsaicin, and the μ-receptor agonist enkephalin, when coadministered into the ventrolateral-PAG at doses nonanalgesic per se, produce 1) antinociception in tests of thermal nociception; 2) stimulation of glutamate release into the RVM; and 3) inhibition of ON neuron activity in the RVM. Both TRPV1 and μ-opioid receptors are coexpressed in several neurons of the VL-PAG. These findings suggest that μ-receptor activation not only acts on inhibitory neurons to disinhibit PAG output neurons but also interacts with TRPV1 activation at increasing glutamate release into the RVM, possibly by acting directly on PAG output neurons projecting to the RVM. Thus, capsaicin can modulate the perception of pain in the brain.

The most frequently encountered adverse effect with capsaicin is burning pain at the site of application, particularly in the first week of application.

In order to make the capsaicins to have less irritation to the skin and significantly less burning sensation to the stomach, the capsaicins have been esterified at the phenolic position. These esters have the general formula I,

R—CO-CAP (I)

wherein CAP refers to collectively the capsaicins represented in FIG. 3.

In formula I, R is selected from alkyl groups of up to about 18 carbon atoms and aryl groups of up to about 18 carbon atoms and alkylene group of up to about 18 carbon atoms and an arylene group of up to about 18 carbon atoms. The alkyl, aryl and alkylene groups may be substituted or unsubstituted, branched or straight chains. In addition, R may contain heteroatoms and may be straight chained or branched.

Examples of suitable straight-chain alkyl groups in formula I include methyl, ethyl, propyl, butyl, hexyl, heptyl, octyl, dodecyl, 1-pentadecyl, 1-heptadecyl and the like groups.

Examples of suitable branched chain alkyl groups in formula I include isopropyl, sec-butyl, t-butyl, 2-methylbutyl, 2-pentyl, 3-pentyl and the like groups.

Examples of suitable cyclic alkyl groups in formula I include cyclopropyl, cyclobutyl, cyclopentyl and cyclohexyl groups.

Examples of suitable “alkenyl” groups in I include vinyl (ethenyl), 1-propenyl, i-butenyl, pentenyl, hexenyl, n-decenyl and c-pentenyl and the like.

The groups may be substituted, generally with 1 or 2 substituents, wherein the substituents are independently selected from halo, hydroxy, alkoxy, amino, mono- and dialkylamino, nitro, carboxyl, alkoxycarbonyl, and cyano groups.

By the expression “phenalkyl groups wherein the alkyl moiety contains 1 to 3 or more carbon atoms” is meant benzyl, phenethyl and phenylpropyl groups wherein the phenyl moiety may be substituted. When substituted, the phenyl moiety of the phenalkyl group may contain independently from 1 to 3 or more alkyl, hydroxy, alkoxy, halo, amino, mono- and dialkylamino, nitro, carboxyl, alkoxycarbonyl and cyano groups.

Examples of suitable “heteroaryl” in formula I are pyridinyl, thienyl or imidazolyl.

As noted herein, the expression “halo” is meant in the conventional sense to include F, Cl, Br, and I.

Among the compounds represented by the general Formula I, preferred compounds are such in which R is one of the following groups: methyl, ethyl, propyl, butyl, pentyl, hexyl, 1-pentadecyl, 1-heptadecyl, isobutyl, methoxyethyl, ethoxyethyl, benzyl and nicotinyl.

The esters of capsaicin can be prepared by any method known to those of ordinary skill in the art. For example, the compounds of the present invention are esters of capsaicin which are the constituents of capsicum. Various methods have been described in the literature pertaining to the synthesis of a number of esters of carboxylic acids and phenols (March's Advanced Organic Chemistry: Reactions, Mechanisms, and Structure, 5th Edition, by Michael B. Smith and Jerry March, John Wiley and Sons, Inc, 2001).

One method that has been utilized for efficient preparation of the ester of capsaicin used in the present invention is through dissolution of the compound in methylene dichloride. Since capsaicin USP27 conatins >95% of capsaicins, to this solution slightly in excess of 1.1 mole equivalent of anhydrous triethylamine is added with stirring at room temperature. To this solution 1 mole equivalent of an acid chloride is added with stirring while keeping the temperature at room temperature. After that, the solution was refluxed for 6-8 hours and stirred for 18-24 hours at room temperature. The organic phase was washed 3-4 times with dilute hydrochloric acid solution in a separating funnel to remove any amine present in the organic solution. The reaction mixture was then washed with equal amount of water three to four times to remove the unreacted amine and its salt in a separating funnel. The organic phase was dried with anhydrous sodium sulfate overnight and the methylene dichloride was removed in a rotary evaporator under vacuum. The resultant oily or vaxy material is called the ester capsaicin as all of the phenols present capsaicin is converted into the corresponding ester.

For oral administration, the preferred ester is the palmitate esters of capsaicins. These esters have less irritation and burning sensation to the stomach and are used for relieving pain through its binding to the VR1 receptors and the depletion of substance P.

Over 325 enzymes are magnesium dependent with many being brain enzymes. Magnesium-deficit modifies the turnover of various types of neurotransmitters including amino acids, nitric oxide, neuropeptides, and cytokines. Intracellular effects of Mg2+ ions are mainly opposite to those of Ca2+ ions, possibly owing to competition at sites where Ca2+ ions activate K+ ion channels. Magnesium-deficiency produces epileptiform activity in the CNS which can be blocked by NMDA-receptor antagonists. Other mechanisms, including alterations in Na+/K(+)-ATPase activity, cAMP/cGMP concentrations and calcium currents in pre- and postsynaptic membranes, may also be at least partially responsible for the neuronal injury associated with low brain magnesium and depression [Morris 1992].

Magnesium-deficiency was found to cause numerous neurological and neuromuscular symptoms including hyperexcitability, depression, pain, behavior disturbances, tetany, headaches, focal seizures, ataxia, anxiety, vertigo, muscular weakness, tremors, irritability, and psychotic behavior, each of which were reversible by magnesium repletion. Hypomagnesemia was also seen in patients with various diseases such as cancer, hepatic cirrhosis, cardiovascular, cerebrovascular disease, and generally poor condition. The most common clinical findings of hypomagnesemia were personality changes and major depression showing that differentiation of brain hypomagnesemia from psychiatric disease is important. Deficits of magnesium result due to inadequate intake or malabsorption and due to the renal loss of magnesium that occurs in certain disease states alcoholism, diabetes) and with drug therapy (antidiuretics, aminoglycosides, fluoroquinolones, cisplatin, digoxin, cyclosporin, amphotericin B).

Most of the brain's regular functions operate quickly and involve the excitatory amino acids glutamate and aspartate in the NMDA receptors. They are involved in NMDA nerve cell electrical conduction activity across brain cell synapses. Learning (long-term potentiation), memory and depression have their foundation in NMDA receptors. Magnesium-depletion is specifically deleterious to neurons by causing NMDA-coupled calcium channels to be biased towards opening, because magnesium is nature's calcium channel blocker. The targets for glutamate binding to NMDA receptors are calcium and magnesium ion channels and to a lesser extent calcium and zinc channel. At normal neuronal resting membrane potentials, pores of the glutamate-gated ion channel are blocked by Mg2+ ions. The ion channel of the NMDA-receptor complex is subject to voltage-dependent regulation by magnesium ions. Normally operating NMDA receptors admit into neurons only the amount of Ca2+ that is vital to their function, but abnormally functioning NMDA receptors increase influx of cellular Ca2+ beyond manageable levels leading to the generation of toxic reactive oxygen species and of toxic amounts of nitric oxide (NO) radicals. It has been shown that NMDA receptor channel characteristics in the dorsal horn are altered by inflammation, and that the changes observed could contribute to the hyperalgesia and allodynia associated with tissue injury. Imbalances in Na+ and Cl− gradients as well as Ca2+ overloading are also implicated in neuronal swelling and cell death, while depolarization of membranes relieves the Mg2+ block and allows Na+ and Ca2+ to enter. Certain drugs can act in place of magnesium including memantine and ketamine with each producing benefits in depression.

It has been shown that the use of Intravenous magnesium (30-mg/kg bolus, 10 mg/kg/h infusion for 48 hours) reduced the use of narcotics in postoperative patients. A double-blind, placebo-controlled, cross-over study was conducted in which magnesium sulphate was administered as an i.v. infusion. Spontaneous pain was recorded and qualitative sensory testing with cotton wool was performed in seven patients with postherpetic neuralgia before and after the i.v. administration of either magnesium sulphate 30 mg kg-1 or saline. During the administration, pain scores were significantly lower for magnesium compared with placebo at 20 and 30 min (P=0.016) but not at 10 min. I.V. magnesium sulphate was safe, well-tolerated and effective in patients with postherpetic neuralgia.

U.S. Pat. No. 6,835,398 discloses a method of treating patients, particularly for pain associated with diseases including erythromelalgia, chronic regional pain syndrome, and reflex sympathetic dystrophy, which involves orally administering high doses of magnesium. The magnesium is introduced through several daily administrations, totaling approximately 2-12 times the RDA for magnesium (600 mg to 5 gm elemental magnesium). These higher levels are achieved through increasing daily dosage amounts gradually in response to patient tolerance and using a more well-tolerated form of magnesium preferably a magnesium solution. Total magnesium intake is divided over several doses per day and taken with copious amounts of water.

Russell (Russell I J, Kamin M, Bennett R M, Schnitzer T J, Green J A, Katz W A (2000): Efficacy of tramadol in treatment of pain in fibromyalgia. J Clin Rheumatol, 6:250-257) used a proprietary tablet “supermalic” (SM), which contains 200 mg of malic acid and 50 mg of Mg, in two sequential trials of 24 subjects. The first trial was a randomized, double-blind, placebo-controlled, crossover study for a two-month period (grade I evidence-based). Patients were randomized to either a fixed dose (three tablets, twice daily) of supermalic or to a placebo for four weeks, followed by a two-week washout and crossover to another four-week treatment period. All clinical assessments were conducted by a single examiner. The results did not show any clear treatment effect attributable to supermalic in the blinded, fixed low-dose trial. Side effects related to long-term exposure to Mg include headache, muscular pain (usually relieved by aspirin), and mild gastrointestinal symptoms.

In almost all the clinical studies involving pain, very high doses of magnesium were required to have any meaningful reduction in pain. These high doses could be used for chronic pain treatment.

### Description of Alternative Ingredients

A non-limiting list of tramadol or its analog drugs which may be utilized in the present invention include any one of (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-desmethyl tramadol”), venlafaxine, (R/S)-1-[2-(dimethylamino)-1-(4-methoxyphenyl)ethyl]cyclohexanol and O-desmethylvenlafaxine or mixtures, stereoisomers, recemates, metabolites, salts or complexes thereof.

A non-limiting list of NMDA antagonist drugs which may be utilized in the present invention include dextromethorphan, magnesium, dextrorphan, ketamine, amantadine, memantine, eliprodil, ifenprodil, phencyclidine, MK-801, dizocilpine, CCPene, flupirtine, or derivatives, salts, metabolites or complexes thereof.

A non-limiting list of analogs of gabapentin which may be used in the present invention include gabapentin, pregabalin, 3-methyl gabapentin, [(1R,5R,6S)-6-(Aminomethyl)bicyclo[-3.2.0]hept-6-yl]acetic acid, 3-(1-Aminomethyl-cyclohexylmethyl)-4H-[1,2,4]-oxadiazol-5-one, C-[1-(1H-Tetrazol-5-ylmethyl)-cycloheptyl]-methylamine, (3S,4S)-(1-Aminomethyl-3,4-dimethyl-cyclopentyl)-acetic acid, (1α,3α,5a)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid and (3S,5R)-3-Amino-5-methyl-octanoic acid, (1-aminomethyl-3-methylcyclohexyl)acetic acid, (1-aminomethyl-3-methylcyclopentyl)acetic acid, (S)-3-(aminomethyl)-5-methylhexanoic acid, 3-aminomethyl-5-methyl-hexanoic acid, (1-aminomethyl-3,4-dimethylcyclopentyl)acetic acid or a pharmaceutically acceptable salt thereof, or an ester or amide derivative thereof.

A non-limiting list of capsaicin which may be used in the present invention include capsaicin itself, homocapsaicin, nordihydrocapsaicin, dihydrocapsaicin, homodihydrocapsaicin or any compounded mixture thereof.

A non-limiting list of pharmaceutically acceptable salt of magnesium which may be used in the present invention include magnesium chloride, magnesium sulfate, magnesium gluconate, magnesium citrate, magnesium aspartate, magnesium lactate, magnesium levulinate, magnesium pidolate, magnesium orotate, magnesium oxide and magnesium malate.

A non-limiting list of an ester of capsaicin which may be used in the present invention includes capsaicin palmitate.

A non-limiting list of a tricyclic anti-depressant which may be used in the present invention includes amitriptyline, butriptyline, amoxapine, clomipramine, desipramine, dothiepin, imipramine, dibenzepin, iprindole, lofepramine, nortriptyline, opipramol, protriptyline, tianeptine, milnacipran and trimipramine.

### Description of Quantitative Pharmacological Parameters of the Mixture

Preferred embodiments of the present invention are pain relieving preparations for oral administration that provide a combination of a NMDA antagonist or a pharmaceutically acceptable salt thereof, an anticonvulsant and/or a tricyclic anti-depressant or a pharmaceutically acceptable salt thereof, and a tramadol or its analog or a pharmaceutically acceptable salt thereof. The combination preferably provides a synergistic or at least additive effect for analgesic dosages.

Dosage levels of the NMDA antagonist on the order of from about 0.3 mg to about 3 mg per kilogram of body weight per day and anticonvulsant and/or a tricyclic anti-depressant on the order of from about 0.05 mg to about 3 mg per kilogram of body weight are therapeutically effective in combination with tramadol or its analog. Alternatively, about 10 mg to about 200 mg per patient per day of a NMDA antagonist and about 5 mg to about 300 mg per patient per day of anticonvulsant and/or a tricyclic anti-depressant are administered in combination with tramadol or its analog. For example, chronic pain may be effectively treated by the administration of from about 0.3 to 3 mg of the NMDA antagonist per kilogram of body weight per day, or alternatively about 30 mg to about 300 mg per patient per day.

The amount of NMDA antagonist that may be combined with the carrier materials to produce a single dosage form having NMDA antagonist, anticonvulsant and/or a tricyclic anti-depressant and tramadol or its analog in combination will vary depending upon the patient and the particular mode of administration. For example, a formulation intended for the oral administration of humans may contain from 10 mg to 300 mg of NMDA antagonist compounded with an appropriate and convenient amount of carrier material that may vary from about 5 to about 95 percent of the total composition. Unit dosages will generally contain between from about 10 mg to about 100 mg of a NMDA antagonist.

Tramadol or its analog can be provided in a sustained release oral dosage form with as the therapeutically active analgesic in an amount from about 25 mg to about 400 mg tramadol hydrochloride. Alternatively, the dosage form may contain molar equivalent amounts of other tramadol salts or of the tramadol base. The dosage form may contain a mixture of tramadol and a derivative of tramadol to provide a substantially equivalent therapeutic effect.

Preferred combinations of the invention comprise an effective amount of a NMDA antagonist selected from the group consisting of dextromethorphan and magnesium, an effective amount tramadol and an effective amount of anticonvulsant and/or a tricyclic anti-depressant.

The amount of anticonvulsant in the composition will be an amount sufficient to further enhance analgesia or to hasten its onset. In humans, this amount will typically be from about 10 to about 3600 mg (preferably 20 to 1000 mg), an amount generally sufficient to both hasten onset and enhance analgesia. The daily dosage of anticonvulsant again will generally not exceed 3600 mg. Of course, greater amounts can be used if tolerated by the patient.

The amount of tricyclic anti-depressant in the composition will be an amount sufficient to further enhance analgesia or to hasten its onset. In humans, this amount will typically be from about 1 to about 1000 mg (preferably 5 to 300 mg), an amount generally sufficient to both hasten onset and enhance analgesia. The daily dosage of tricyclic anti-depressant again will generally not exceed 300 mg. Of course, greater amounts can be used if tolerated by the patient.

The amount of capsaicin palmitate in the composition will be an amount sufficient to further enhance analgesia or to hasten its onset. In humans, this amount will typically be from about 1 to about 100 mg (preferably 5 to 30 mg), an amount generally sufficient to both hasten onset and enhance analgesia. The daily dosage of capsaicin palmitate again will generally not exceed 100 mg. Of course, greater amounts can be used if tolerated by the patient.

In certain preferred embodiments according to the present invention, an oral dosage form is preferred which includes the following tramadol or its analog/NMDA antagonist/anticonvulsant combinations: Tramadol 35 mg plus 45 mg dextromethorphan plus 90 mg gabapentin; tramadol 35 mg plus 45 mg dextromethorphan plus 180 mg gabapentin; tramadol 35 mg plus 45 mg dextromethorphan plus 45 mg gabapentin or 50 mg of tramadol plus 30 mg of dextromethorphan plus 90 mg gabapentin; Tramadol mg plus 45 mg dextromethorphan plus 90 mg gabapentin.

In another preferred embodiments according to the present invention, an oral dosage form is preferred which includes the following tramadol or its analog/NMDA antagonist/anticonvulsant/capsaicin palmitate combinations: Tramadol 35 mg plus 45 mg dextromethorphan plus 90 mg gabapentin plus 5.4 mg of capsaicin palmitate; tramadol 35 mg plus 45 mg dextromethorphan plus 180 mg gabapentin plus 5.4 mg of capsaicin palmitate; tramadol 35 mg plus 45 mg dextromethorphan plus 45 mg gabapentin plus 10.8 mg of capsaicin palmitate; 50 mg of tramadol plus 30 mg of dextromethorphan plus 90 mg gabapentin plus 10.8 mg of capsaicin palmitate.

In certain preferred embodiments according to the present invention, an oral dosage form is preferred which includes the following tramadol or its analog/NMDA antagonist/tricyclic antidepressant combinations: Tramadol 35 mg plus 45 mg dextromethorphan plus 10 mg amitriptyline or milnacipran; tramadol 35 mg plus 45 mg dextromethorphan plus 5 mg amitriptyline or milnacipran; or 50 mg of tramadol plus 30 mg of dextromethorphan plus 10 mg amitriptyline or milnacipran.

In certain preferred embodiments according to the present invention, an oral dosage form is preferred which includes the following tramadol or its analog/NMDA antagonist/anticonvulsant and tricyclic antidepressant combinations: Tramadol 35 mg plus 45 mg dextromethorphan plus 90 mg gabapentin plus 10 mg amitriptyline or milnacipran; tramadol 35 mg plus 45 mg dextromethorphan plus 45 mg gabapentin plus 5 mg amitriptyline or milnacipran; tramadol 35 mg plus 45 mg dextromethorphan plus 45 mg gabapentin plus 10 mg amitriptyline or milnacipran; or 35 mg of tramadol plus 30 mg of dextromethorphan plus 90 mg gabapentin plus 10 mg amitriptyline or milnacipran.

In another preferred embodiments according to the present invention, an oral dosage form is preferred which includes the following tramadol or its analog/NMDA antagonist/anticonvulsant combinations: Tramadol 50 mg plus 20.4 mg magnesium plus 100 mg gabapentin; tramadol 35 mg plus 40.8 mg magnesium plus 100 mg gabapentin.

Yet in another preferred embodiments according to the present invention, an oral dosage form is preferred which includes the following tramadol or its analog/NMDA antagonist/tricyclic antidepressant/capsaicin palmitate combinations: Tramadol 35 mg plus 45 mg dextromethorphan plus 10 mg amitriptyline or milnacipran plus 5.4 mg of capsaicin palmitate; tramadol 35 mg plus 45 mg dextromethorphan plus 10 mg amitriptyline or milnacipran plus 10.8 mg of capsaicin palmitate; tramadol 35 mg plus 30 mg dextromethorphan plus 10 mg amitriptyline or milnacipran plus 10.8 mg of capsaicin palmitate; 50 mg of tramadol plus 30 mg of dextromethorphan plus 10 mg amitriptyline or milnacipran plus 10.8 mg of capsaicin palmitate; Tramadol 44 mg plus 20.4 mg magnesium plus 100 mg gabapentin plus 5.4 mg of capsaicin palmitate.

The dosage administered will of course vary depending upon known factors such as the pharmacodynamic characteristics of each agent of the combination and its mode and route of administration and upon the age, health and weight of the patient. The dosage will also depend upon the nature and extent of symptoms, concurrent treatment, if any, frequency of treatment and the desired result. A composition comprising any of the above identified combinations of tramadol or its analog, gabapentin or analog of gabapentin and NMDA antagonist may be administered in divided doses ranging from 2 to 6 times per day or in a sustained release form that will provide a rate of release effective to attain the desired results.

The optimal NMDA antagonist to tramadol or its analog ratios can be determined by standard assays well known in the art for determining opiate and analgesic activity. For example, the phenyl-p-benzoquinone test may be used to establish analgesic effectiveness. The phenyl-p-benzoquinone induced writhing test in mice as described in Blumberg et al, 1965, Proc. Soc. Exp. Med. 118:763-766, hereby incorporated by reference, and known modifications thereof, is a standard procedure which may be used for detecting and comparing the analgesic activity of different classes of analgesic drugs with a good correlation with human analgesic activity. Data for the mouse, as presented in an isobologram, can be translated to other species where the orally effective analgesic dose of the individual compounds are known or can be estimated.

The synergistic effect of the combination can further be appreciated using Table 1. As can be seen, the administration of tramadol 50 mg plus 20.4 mg magnesium plus 100 mg gabapentin twice daily, reduces the pain in fibromyalgia and diabetic neuropathy patients by almost 95% while the individual components at these doses would have very little effect on the pain.

TABLE 1
The synergistic Effect of the Combination Therapy

Pain Relief
(percent of reduction in pain)

| | Dose | Diabetic | |
|---|---|---|---|
| Compound | mg/day | Neuropathy | Fibromyalgia |
| Magnesium | 400-600 | (Brill 2002) | |
| | | Post-herpetic | |
| | | Neuralgia | |
| | | iv infusion | |
| Tramadol | 400 | <35 | <30 |
| Hydrochloride | | (Sindrup | (Biasi 1998; |
| | | 1999) | Russell 2000) |
| Gabapentin | 3600 | <50 | <50 |
| | | (Marchettini | (Lesley 2007) |
| | | 2004) | |
| | | 80-95 | 80-95 |
| | | (N = 3) | (N = 3) |
| TLI-0326 | 40.4 | <10 | <10 |
| Magnesium (present | 100 | <10 | <10 |
| as magnesium | | | |
| sulfate) | | | |
| Tramadol (present | 200 | <10 | <10 |
| as Hydrochloride) | | | |
| Gabapentin | | | |

### Elaboration of Preferred and Alternative Formulations and Vehicles

The present invention encompasses immediate release dosage forms of an effective analgesic amount of dextromethorphan, gabapentin or an analog of gabapentin and tramadol or its analog combination. An immediate release dosage form may be formulated as a tablet or multi-particulate that may be encapsulated. Other immediate release dosage forms known in the art can be employed.

Compositions of the invention present the opportunity for obtaining relief from moderate to severe pain. Due to the synergistic and/or additive effects provided by the inventive combination of tramadol or its analog, anticonvulsant and/or a tricyclic anti-depressant and NMDA antagonist, it may be possible to use reduced dosages of each of NMDA antagonist and tramadol or its analog. By using lesser amounts of other or both drugs, the side effects associated with each may be reduced in number and degree. Moreover, the inventive combination avoids side effects to which some patients are particularly sensitive.

The present invention encompasses a method of inhibiting NMDA receptor and treating diseases comprising administering to a patient in need of such treatment a non-toxic therapeutically effective amount of the NMDA antagonist, anticonvulsant and/or a tricyclic anti-depressant and tramadol or its analog combination of the present invention. These diseases include, but not limited to, moderate to severe pain arising from many different etiologies, including but not limited to, cancer pain and post-surgical pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis, degenerative joint diseases such as osteoarthritis, gout and ankylosing spondylitis, bursitis, burns, migraine headache, fibromyalgia syndrome, multiple sclerosis syndrome, trigeminal neuralgia, symptoms associated with diabetic neuropathy and injuries.

Further, the combination of NMDA antagonist, anticonvulsant and/or a tricyclic anti-depressant and tramadol or its analog is useful as an alternative to conventional non-steroidal anti-inflammatory drugs or combinations of NSAIDS with other drugs particularly where such non-steroidal anti-inflammatory drugs may be contraindicated such as in patients with peptic ulcers, gastritis, regional enteritis, ulcerative colitis, diverticulitis or with a recurrent history of gastrointestinal lesions, GI bleeding, coagulation disorders including anemia such as hypoprothrombinemia, haemophilia or other bleeding problems, kidney disease and in those prior to surgery or taking anticoagulants.

The sustained release dosage forms of the present invention generally achieve and maintain therapeutic levels substantially without significant increases in the intensity and/or degree of concurrent side effects, such as nausea, vomiting, seizures or drowsiness, which are often associated with high blood levels of tramadol or its analogs. There is also evidence to suggest that the use of the present dosage forms leads to a reduced risk of drug addiction.

The combination of NMDA antagonist, anticonvulsant and/or a tricyclic anti-depressant and tramadol or its analog may be formulated to provide for an increased duration of analgesic action allowing once daily dosing. Similarly, the combination of NMDA antagonist, capsaicin or an ester of capsaicin, anticonvulsant and/or a tricyclic anti-depressant and tramadol or its analog may be formulated to provide for an increased duration of analgesic action allowing once daily dosing. These formulations, at comparable daily dosages of conventional immediate release drug, are associated with a lower incidence in severity of adverse drug reactions and can also be administered at a lower daily dose than conventional oral medication while maintaining pain control.

The combination of NMDA antagonist, anticonvulsant and/or a tricyclic anti-depressant and tramadol or its analog or the combination of NMDA antagonist, anticonvulsant and/or a tricyclic anti-depressant, capsaicin or an ester of capsaicin and tramadol or its analog can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For parenteral application, particularly suitable are oily or aqueous solutions, as well as suspensions, emulsions, or implants, including suppositories. For oral application, particularly suitable are tablets, troches, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose, granulating and disintegrating agents such as cornstarch, binding agents such as starch, and lubricating agents such as magnesium stearate. The tablets may be uncoated or they may be coated by known techniques for elegance or to delay release of the active ingredients. Formulations for oral use may also be presented as hard gelatin capsules wherein the active ingredient is mixed with an inert diluent.

Aqueous suspensions that contain the aforementioned combinations of drugs and that such a mixture has one or more excipients suitable as suspending agents, for example pharmaceutically acceptable synthetic gums such as hydroxypropylmethylcellulose or natural gums. Oily suspensions may be formulated by suspending the aforementioned combinations of drugs in a vegetable oil or mineral oil. The oily suspensions may contain a thickening agent such as bees' wax or cetyl alcohol. A syrup, elixir, or the like can be used wherein a sweetened vehicle is employed. Injectable suspensions may also be prepared, in which case appropriate liquid carriers, suspending agents and the like may be employed. It is also possible to freeze-dry the active compounds and use the obtained lyophilized compounds, for example, for the preparation of products for injection.

The method of treatment and pharmaceutical formulations of the present invention may further include one or more drugs in addition to a NMDA antagonist, an anticonvulsant and/or a tricyclic anti-depressant and tramadol or its analog, which additional drug(s) may or may not act synergistically therewith. Examples of such additional drugs include, but not limited to, vanilloid receptor antagonists, NSAIDs, including ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, acetaminophen and the like.

### Controlled Release Dosage Forms

The NMDA antagonist, anticonvulsant and/or a tricyclic anti-depressant and tramadol or its analog combination or the NMDA antagonist, anticonvulsant and/or a tricyclic anti-depressant, capsaicin or an ester of capsaicin and tramadol or its analog combination can be formulated as a controlled or sustained release oral formulation in any suitable tablet, coated tablet or multiparticulate formulation known to those skilled in the art. The sustained release dosage form may optionally include a sustained released carrier which is incorporated into a matrix along with tramadol or its analog, or which is applied as a sustained release coating.

The sustained release dosage form may include the tramadol or its analog in sustained release form and the NMDA antagonist and anticonvulsant and/or a tricyclic anti-depressant in sustained release form or in immediate release form. The NMDA antagonist and anticonvulsant and/or a tricyclic anti-depressant may be incorporated into the sustained release matrix along with tramadol or its analog, incorporated into the sustained release coating; incorporated as a separated sustained release layer or immediate release layer, or may be incorporated as a powder, granulation, etc., in a gelatin capsule with the substrates of the present invention. Alternatively, the sustained release dosage form may have the NMDA antagonist in sustained release form and the tramadol or its analog and anticonvulsant and/or a tricyclic anti-depressant in sustained release form or immediate release form.

An oral dosage form according to the invention may be provided as, for example, granules, spheroids, beads, and pellets or pills. These formulations are hereinafter collectively referred to as “multiparticulates” and/or particles. An amount of the multiparticulates that is effective to provide the desired dose of tramadol or its analog over time may be placed in a capsule or may be incorporated in any other suitable oral solid form.

In one preferred embodiment of the present invention, the sustained release dosage form comprises such particles containing or comprising the active ingredient, wherein the particles have diameter from about 0.1 mm to about 2.5 mm, preferably from about 0.5 mm to about 2 mm.

In certain embodiments, the particles comprise normal release matrixes containing the tramadol or its analog with or without the NMDA antagonist and anticonvulsant and/or a tricyclic anti-depressant. These particles are then coated with the sustained release carrier. In embodiments where the NMDA antagonist and anticonvulsant and/or a tricyclic anti-depressant are immediately released, the NMDA antagonist and anticonvulsant and/or a tricyclic anti-depressant may be included in separate normal release matrix particles, or may be co-administered in a different immediate release composition which is either enveloped within a gelatin capsule or is administered separately. In other embodiments, the particles comprise inert beads that are coated with tramadol or its analog with or without the NMDA antagonist and anticonvulsant and/or a tricyclic anti-depressant. Thereafter, a coating comprising the sustained release carrier is applied onto the beads as an overcoat.

The particles are preferably film coated with a material that permits release of the tramadol or its analog or its salt, and if desired, the NMDA antagonist and anticonvulsant and/or a tricyclic anti-depressant at a sustained rate in an aqueous medium. The film coat is chosen so as to achieve, in combination with the other stated properties, a desired in vivo release rate. The sustained release coating formulations of the present invention should be capable of producing a strong, continuous film that is smooth and elegant, capable of supporting pigments and other coating additives, non-toxic, inert, and tack free.

### Coatings

The dosage forms of the present invention may optionally be coated with one or more materials suitable for the regulation of release or for the protection of the formulation. In one embodiment, coatings are provided to permit either pH dependent or pH independent release, e.g., when exposed to gastrointestinal fluid. A pH dependent coating serves to release the tramadol or its analog in desired areas of the gastro-intestinal (GI) tract, e.g., the stomach or small intestine, such that an absorption profile is provided which is capable of providing at least about twelve hour and preferably up to twenty four hour analgesia to a patient. When a pH independent coating is desired, the coating is designed to achieve optimal release regardless of pH changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Formulations according to the invention that utilize pH dependent coatings to obtain formulations may also impart a repeat-action or pulsatile release effect whereby unprotected drug is coated over the enteric coat and is released in the stomach, while the remainder, being protected by the enteric coating, is released further down the gastrointestinal tract. Coatings which are pH dependent may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like.

The substrate (e.g., tablet core bead, matrix particle) containing the tramadol or its analog (with or without the NMDA antagonist and anticonvulsant and/or a tricyclic anti-depressant) is coated with a hydrophobic material selected from (i) an alkylcellulose; (ii) an acrylic polymer, or (iii) mixtures thereof. The coating may be applied in the form of an organic or aqueous solution or dispersion. The coating may be applied to obtain a weight gain from about 2 to about 25% of the substrate in order to obtain a desired sustained release profile. Such formulations are described in detail in U.S. Pat. Nos. 5,273,760 and 5,286,493, hereby incorporated by reference in their entirety.

Other examples of sustained release formulations and coatings that may be used in accordance with the present invention include U.S. Pat. Nos. 5,324,351, 5,356,467, and 5,472,712, hereby incorporated by reference in their entirety.

### Alkylcellulose Polymers

Cellulosic materials and polymers, including alkylcelluloses, provide hydrophobic materials well suited for coating the beads according to the invention. Simply by way of example, one preferred alkylcellulosic polymer is ethylcellulose, although the artisan will appreciate that other cellulose and/or alkylcellulose polymers may be readily employed, singly or in any combination, as all or part of a hydrophobic coating according to the invention.

One commercially available aqueous dispersion of ethylcellulose is sold as Aquacoat™ (FMC Corp., Philadelphia, Pa., U.S.A.). Aquacoat™ is prepared by dissolving the ethylcellulose in a water immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudo-latex during the manufacturing phase. Thus, prior to using the same as a coating, it is necessary to intimately mix the Aquacoat™ with a suitable plasticizer prior to use.

Another aqueous dispersion of ethylcellulose is commercially available as Surelease™ (Colorcon, Inc., West Point, Pa., U.S.A.). This product is prepared by incorporating plasticizer into the dispersion during the manufacturing process. A hot melt of a polymer containing for example a plasticizer such as dibutyl sebacate, and a stabilizer such as oleic acid is prepared as a homogeneous mixture, which is then diluted with an alkaline solution to obtain an aqueous dispersion which can be applied directly onto substrates.

### Acrylic Polymers

The hydrophobic material comprising the controlled release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers. The acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral methacrylic esters.

Certain methacrylic acid ester type polymers are useful for preparing pH dependent coatings that may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit™ from Rohm Tech, Inc. There are several different types of Eudragit™. For example Eudragit™ E is an example of a methacrylic acid copolymer that swells and dissolves in acidic media. Eudragit™ L is a methacrylic acid copolymer which does not swell at about pH<5.7 and is soluble at about pH>6. Eudragit™ S does not swell at about pH<6.5 and is soluble at about pH>7. Eudragit™ L and Eudragit™ S are water swellable, and the amount of water absorbed by these polymers is pH dependent. However, dosage forms coated with Eudragit™ L and S are pH independent.

The acrylic coating comprises a mixture of two acrylic resin lacquers commercially available from Rohm Pharma under the Tradenames Eudragit™ L30D and Eudragit™ S30D, respectively. Eudragit™ L30D and Eudragit™ S30D are copolymers of acrylic and methacrylic esters with a low content of quaternary ammonium groups, the molar ratio of ammonium groups to the remaining neutral methacrylic esters being 1:20 in Eudragit™ L30D and 1:40 in Eudragit™ S30D. The mean molecular weight is about 150,000. The code designations RL (high permeability) and RS (low permeability) refer to the permeability properties of these agents. Eudragit™ RL/RS mixtures are insoluble in water and in digestive fluids. However, coatings formed from the same are swellable and permeable in aqueous solutions and digestive fluids.

The Eudragit™ RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit™ RL, 50% Eudragit™ RL and 50% Eudragit™ RS, and 10% Eudragit™ RL Eudragit™ 90% RS. Of course, one skilled in the art will recognize that other acrylic polymers may also be used, such as, for example, Eudragit™ L.

### Plasticizers

In the present invention where the coating comprises an aqueous dispersion of a hydrophobic material, the inclusion of an effective amount of a plasticizer in the aqueous dispersion of hydrophobic material will further improve the physical properties of the sustained release coating. For example, because ethylcellulose has a relatively high glass transition temperature and does not form flexible films under normal coating conditions, it is preferable to incorporate a plasticizer into an ethylcellulose coating containing sustained release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers, such as acetylated monoglycerides, phthalate esters, castor oil, etc., may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate, tributyl citrate, dibutyl phthalate, and possibly 1,2-propylene glycol. Other plasticizers that have proved to be suitable for enhancing the elasticity of the films formed from acrylic films such as Eudragit™ RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that the addition of a small amount of talc reduces the tendency of the aqueous dispersion to stick during processing, and acts as a polishing agent.

### Processes for Preparing Coated Beads

When the aqueous dispersion of hydrophobic material is used to coat inert pharmaceutical beads such as nu-pariel 18/20 beads, a plurality of the resultant stabilized solid controlled release beads may thereafter be placed in a gelatin capsule in an amount sufficient to provide an effective controlled release dose when ingested and contacted by an environmental fluid, e.g., gastric fluid or dissolution media.

The stabilized controlled release bead formulations of the present invention slowly release the therapeutically active agent, e.g., when ingested and exposed to gastric fluids, and then to intestinal fluids. The controlled release profile of the formulations of the invention can be altered, for example, by varying the amount of overcoating with the aqueous dispersion of hydrophobic material, altering the manner in which the plasticizer is added to the aqueous dispersion of hydrophobic material, by varying the amount of plasticizer relative to hydrophobic material, by the inclusion of additional ingredients or excipients, by altering the method of manufacture, etc. The payload release profile of the product may also be modified by increasing or decreasing the thickness of the retardant coating.

Spheroids or beads coated with a therapeutically active agent are prepared, e.g., by dissolving the therapeutically active agent in water and then spraying the solution onto a substrate, for example, nu pariel 18/20 beads, using a Wuster insert. Optionally, additional ingredients are also added prior to coating the beads in order to assist the binding of the tramadol or its analog to the beads, and/or to color the solution, etc. For example, a product that includes hydroxypropylmethylcellulose, etc. with or without a colorant, such as Opadry™, commercially available from Colorcon, Inc., may be added to the solution and the solution mixed for about 1 hour prior to application of the same onto the beads. The resultant coated substrate, in this example beads, may then be optionally overcoated with a barrier agent, to separate the therapeutically active agent from the hydrophobic controlled release coating. An example of a suitable barrier agent is one that comprises hydroxypropylmethylcellulose. However, any film former known in the art may be used. It is preferred that the barrier agent does not affect the dissolution rate of the final product.

The beads may then be overcoated with an aqueous dispersion of the hydrophobic material. The aqueous dispersion of hydrophobic material preferably further includes an effective amount of plasticizer, e.g. triethyl citrate. Pre-formulated aqueous dispersions of ethylcellulose, such as Aquacoat™ or Surelease™, may be used. If Surelease™ is used, it is not necessary to separately add a plasticizer. Alternatively, pre-formulated aqueous dispersions of acrylic polymers such as Eudragit™ can be used.

The coating solutions of the present invention preferably contain, in addition to the film former, plasticizer, and solvent system such as water and a colorant to provide elegance and product distinction. Color may be added to the solution of the therapeutically active agent instead, or in addition to the aqueous dispersion of hydrophobic material. For example, color be added to Aquacoat™ via the use of alcohol or propylene glycol based color dispersions, milled aluminum lakes and opacifiers such as titanium dioxide by adding color with shear to water soluble polymer solution and then using low shear to the plasticized Aquacoat™. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the release retarding effect of the coating.

The plasticized aqueous dispersion of hydrophobic material may be applied onto the substrate comprising the therapeutically active agent by spraying using any suitable spray equipment known in the art. In a preferred method, a Wurster fluidized bed system is used in which an air jet, injected from underneath, fluidizes the core material and effects drying while the acrylic polymer coating is sprayed on. A sufficient amount of the aqueous dispersion of hydrophobic material to obtain a predetermined controlled release of said therapeutically active agent when said coated substrate is exposed to aqueous solutions, such as gastric fluid, is preferably applied, taking into account the physical characteristics of the therapeutically active agent, the manner of incorporation of the plasticizer, etc. After coating with the hydrophobic material, a further overcoat of a film-former, such as Opadry™, is optionally applied to the beads. This overcoat is provided, if at all, in order to substantially reduce agglomeration of the beads.

The release of the therapeutically active agent from the controlled release formulation of the present invention can be further influenced and adjusted to a desired rate by the addition of one or more release modifying agents. Controlled release may be achieved in the alternative by providing one or more passageways through the coating through which the drug or a solution of the drug can diffuse. The ratio of hydrophobic material to water soluble material is determined by, among other factors, the release rate required to produce the desired therapeutic effect and the solubility characteristics of the materials selected.

The release modifying agents which function as pore formers may be organic or inorganic, and include materials that can be dissolved, extracted or leached from the coating in the environment of use. The pore-formers may comprise one or more hydrophilic materials such as hydroxypropylmethylcellulose.

The sustained release coatings of the present invention can also include erosion promoting agents such as starches and gums.

The sustained release coatings of the present invention can also include materials useful for making microporous lamina in the environment of use, such as polycarbonates comprised of linear polyesters of carbonic acid in which carbonate groups reoccur in the polymer chain. The release modifying agent may also comprise a semi-permeable polymer.

The release modifying agent can be preferably selected from hydroxypropylmethylcellulose, lactose, metal stearates, and mixtures of any of the foregoing.

The sustained release coatings of the present invention may also include an exit means comprising at least one passageway, orifice, or the like. The passageway may be formed by such methods as those disclosed in U.S. Pat. Nos. 3,845,770, 3,916,889, 4,063,064 and 4,088,864, all of which are hereby incorporated by reference. The passageway can have any shape such as round, triangular, square, elliptical, irregular, etc.

### Matrix Bead Formulations

The controlled release formulation is achieved via a matrix having a controlled release coating as set forth above. The present invention may also utilize a controlled release matrix that affords in vitro dissolution rates of the tramadol or its analog within the preferred ranges and that releases the tramadol or its analog in a pH dependent or pH independent manner. The materials suitable for inclusion in a controlled release matrix will depend on the method used to form the matrix.

For example, a matrix in addition to the tramadol or its analog and, optionally, a NMDA antagonist and an anticonvulsant and/or a tricyclic anti-depressant may include:

Hydrophilic and/or hydrophobic materials, such as gums, cellulose ethers, acrylic resins, protein derived materials; the list is not meant to be exclusive, and any pharmaceutically acceptable hydrophobic material or hydrophilic material which is capable of imparting controlled release of the active agent and which melts or softens to the extent necessary to be extruded may be used in accordance with the present invention.

Digestible, long chain (C₈ to C₅₀, especially C₁₂ to C₄₀), substituted or unsubstituted hydrocarbons, such as fatty acids, fatty alcohols, glyceryl esters of fatty acids, mineral and vegetable oils and waxes, and stearyl alcohol; and polyalkylene glycols.

Of these polymers, acrylic polymers, especially Eudragit™, RSPO, the cellulose ethers, especially hydroxyalkylcelluloses and carboxyalkylcelluloses, are preferred. The oral dosage form may contain between 1% and 80% by weight of at least one hydrophilic or hydrophobic material.

When the hydrophobic material is a hydrocarbon, the hydrocarbon preferably has a melting point of between 25 and 90 carbon atoms. Of the long chain hydrocarbon materials, fatty aliphatic alcohols are preferred. The oral dosage form may contain up to 60% (by weight) of at least one digestible, long chain hydrocarbon.

Preferably, the oral dosage form contains up to 60% by weight of at least one polyalkylene glycol.

The hydrophobic material is preferably selected from the group consisting of alkylcelluloses, acrylic and methacrylic acid polymers and copolymers, shellac, zein, hydrogenated castor oil, hydrogenated vegetable oil, or mixtures thereof. In certain preferred embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cynaoethyl methacrylate, aminoalkyl methacrylate copolymer, polyacrylic acid, polymethacrylic acid, methacrylic acid alkylamine copolymer, polymethyl methacrylate, polymethacrylic acid anhydride, polymethacrylate, polyacrylamide, poly(methacrylic acid anhydride), and glycidyl methacrylate copolymers. In other embodiments, the hydrophobic material is selected from materials such as hydroxyalkylcelluloses such as hydroxypropylmethylcellulose and mixtures of the foregoing.

Preferred hydrophobic materials are water-insoluble with more or less pronounced hydrophilic and/or hydrophobic trends. Preferably, the hydrophobic materials useful in the invention have a melting point from about 30 to about 200° C., preferably from about 45 to about 90° C. Specifically, the hydrophobic material may comprise natural or synthetic waxes, fatty alcohols such as lauryl, myristyl, stearyl, cetyl or preferably cetostearyl alcohol, fatty acids, including but not limited to fatty acid esters, fatty acid glycerides (mono-, di-, and tri-glycerides), hydrogenated fats, hydrocarbons, normal waxes, stearic aid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material that is normally solid at room temperature and has a melting point of from about 30 to about 100° C.

Suitable hydrophobic materials which may be used in accordance with the present invention include digestible, long chain (C₈ to C₅₀, especially C₁₂ to C₄₀), substituted or unsubstituted hydrocarbons, such as fatty acids, fatty alcohols, glyceryl esters of fatty acids, mineral and vegetable oils and natural and synthetic waxes. Hydrocarbons having a melting point of between 25 and 90° C. are preferred. Of the long chain hydrocarbon materials, fatty (aliphatic) alcohols are preferred in certain embodiments. The oral dosage form may contain up to 60% by weight of at least one digestible, long chain hydrocarbon.

Preferably, a combination of two or more hydrophobic materials is included in the matrix formulations. If an additional hydrophobic material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable matrix comprises at least one water soluble hydroxyalkyl cellulose, at least one C₁₂ to C₃₆, preferably C₁₄ to C₂₂, aliphatic alcohol and, optionally, at least one polyalkylene glycol. The at least one hydroxyalkyl cellulose is preferably a hydroxy (C₁ to C₆) alkyl cellulose, such as hydroxypropylcellulose, hydroxypropylmethylcellulose and, especially, hydroxyethylcellulose. The amount of the at least one hydroxyalkyl cellulose in the present oral dosage form will be determined, inter alia, by the precise rate of tramadol or its analog release required. The at least one aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of the at least one aliphatic alcohol in the present oral dosage form will be determined, as above, by the precise rate of tramadol or its analog release required. It will also depend on whether at least one polyalkylene glycol is present in or absent from the oral dosage form. In the absence of at least one polyalkylene glycol, the oral dosage form preferably contains between 20% and 50% by weight of the at least one aliphatic alcohol. When at least one polyalkylene glycol is present in the oral dosage form, then the combined weight of the at least one aliphatic alcohol and the at least one polyalkylene glycol preferably constitutes between 20% and 50% by weight of the total dosage.

The ratio of hydroxyalkyl cellulose or acrylic resin to the aliphatic alcohol/polyalkylene glycol determines, to a considerable extent, the release rate of the tramadol or its analog from the formulation. A ratio of the hydroxyalkyl cellulose to the aliphatic alcohol/polyalkylene glycol of between 1:2 and 1:4 is preferred, with a ratio of between 1:3 and 1:4 being particularly preferred.

The polyalkylene glycol may be, for example, polypropylene glycol or, which is preferred, polyethylene glycol. The number average molecular weight of the polyalkylene glycol is preferred between 1,000 and 15,000 especially between 1,500 and 12,000.

Another suitable controlled release matrix would comprise an alkylcellulose, especially ethyl cellulose, a C₁₂ to C₃₆ aliphatic alcohol and optionally a polyalkylene glycol.

The preferred matrix includes a pharmaceutically acceptable combination of at least two hydrophobic materials.

In addition to the above ingredients a controlled release matrix may also contain suitable quantities of other materials, for example diluents, lubricants, binders, granulating aids, colorants, flavorants and glidants that are conventionally used in the art of pharmaceutical formulation.

### Processes for Preparing Matrix Based Beads

In order to facilitate the preparation of a solid, controlled release, oral dosage form according to this invention, any method of preparing a matrix formulation known to those skilled in the art may be used. For example incorporation in the matrix may be effected, for example, by (a) forming granules comprising at least one water soluble hydroxyalkyl cellulose and tramadol or its analog or a tramadol or its analog salt; (b) mixing the hydroxyalkyl cellulose containing granules with at least one C₁₂ to C₃₆ aliphatic alcohol; and (c) optionally, compressing and shaping the granules. Preferably, the granules are formed by wet granulating the hydroxyalkyl cellulose/tramadol or its analog with water. In a particularly preferred embodiment of this process, the amount of water added during tie wet granulation step is preferably between 1.5 and 5 times, especially between 1.75 and 3.5 times, the dry weight of the tramadol or its analog.

A spheronizing agent, together with the active ingredient can be spheronized to form spheroids. Microcrystalline cellulose is preferred. A suitable microcrystalline cellulose is, for example, the material sold as Avicel PH 101™ (FMC Corporation). In such embodiments, in addition to the active ingredient and spheronizing agent, the spheroids may also contain a binder. Suitable binders, such as low viscosity water soluble polymers, will be well known to those skilled in the pharmaceutical arts. However water soluble hydroxy lower alkyl cellulose, such as hydroxypropylcellulose are preferred. Additionally, or alternatively, the spheroids may contain a water insoluble polymer, especially an acrylic polymer, an acrylic copolymer, such as a methacrylic acid-ethyl acrylate copolymer, or ethyl cellulose. In such embodiments, the sustained release coating will generally include a hydrophobic material such as (a) a wax, either alone or in admixture with a fatty alcohol, or (b) shellac or zein.

### Melt Extrusion Matrix

Sustained release matrices can also be prepared via melt-granulation or melt-extrusion techniques. Generally, melt-granulation techniques involve melting a normally solid hydrophobic material, such as a wax, and incorporating a powdered drug therein. To obtain a sustained release dosage form, it may be necessary to incorporate an additional hydrophobic substance, such as ethylcellulose or a water insoluble acrylic polymer, into the molten wax hydrophobic material. Examples of sustained release formulations prepared by melt granulation techniques as are found in U.S. Pat. No. 4,861,598, assigned to the Assignee of the present invention and hereby incorporated by reference in its entirety.

The additional hydrophobic material may comprise one or more water-insoluble wax like thermoplastic substances possibly mixed with one or more wax like thermoplastic substances being less hydrophobic than said one or more water insoluble wax like substances. In order to achieve constant release, the individual wax like substances in the formulation should be substantially non-degradable and insoluble in gastrointestinal fluids during the initial release phases. Useful water-insoluble wax like substances may be those with a water solubility that is lower than about 1:5,000 (w/w).

In addition to the above ingredients, a sustained release matrix may also contain suitable quantities of other materials, such as diluents, lubricants, binders, granulating aids, colorants, flavorants and glidants that are conventionally used in the pharmaceutical arts. The quantities of these additional materials will be sufficient to provide the desired effect to the desired formulation. In addition to the above ingredients, a sustained release matrix incorporating melt-extruded multiparticulates may also contain suitable quantities of other materials, such as diluents, lubricants, binders, granulating aids, colorants, flavorants and glidants that are conventional in the pharmaceutical art in amounts up to about 50% by weight of the particulate if desired.

Specific examples of pharmaceutically acceptable carriers and excipients that may be used to formulate oral dosage forms are described in the Handbook of Pharmaceutical Excipients, American Pharmaceutical Association (1986), incorporated by reference herein.

### Melt Extrusion Multiparticulates

The preparation of a suitable melt-extruded matrix according to the present invention may, for example, include the steps of blending tramadol or its analog, together with at least one hydrophobic material and preferably the additional hydrophobic material to obtain a homogeneous mixture. The homogeneous mixture is then heated to a temperature sufficient to at least soften the mixture sufficiently to extrude the same. The resulting homogeneous mixture is then extruded to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides sustained release of the therapeutically active agent for a time period of from about 8 to about 24 hours.

An optional process for preparing the melt extrusions of the present invention includes directly metering into an extruder a hydrophobic material, a therapeutically active agent, and an optional binder, heating the homogenous mixture; extruding the homogenous mixture to thereby form strands; cooling the strands containing the homogeneous mixture, cutting the strands into particles having a size from about 0.1 mm to about 12 mm, and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic material as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

The oral dosage forms can be prepared to include an effective amount of melt-extruded multiparticulates within a capsule. For example, a plurality of the melt-extruded multiparticulates may be placed in a gelatin capsule in an amount sufficient to provide an effective sustained release dose when ingested and contacted by gastric fluid.

A suitable amount of the multiparticulate extrudate can be compressed into an oral tablet using conventional tableting equipment using standard techniques. Techniques and compositions for making tablets that are compressed and/or molded, capsules of hard and soft gelatin, and pills are also described in Remington's Pharmaceutical Sciences, (Arthur Osol, editor), 1553-1593 (1980), incorporated by reference herein.

The extrudate can be shaped into tablets as set forth in U.S. Pat. No. 4,957,681, (Klimesch, et al), described in additional detail above and hereby incorporated by reference.

Optionally, the sustained release melt-extruded multiparticulate systems or tablets can be coated, or the gelatin capsule can be further coated, with a sustained release coating such as the sustained release coatings described above. Such coatings preferably include a sufficient amount of hydrophobic material to obtain a weight gain level from about 2 to about 30 percent, although the overcoat may be greater depending upon the physical properties of the particular tramadol or its analog compound utilized and the desired release rate, among other things.

The melt extruded unit dosage forms of the present invention may further include combinations of melt extruded multiparticulates containing one or more of the therapeutically active agents disclosed above before being encapsulated. Furthermore, the unit dosage forms can also include an amount of an immediate release therapeutically active agent for prompt therapeutic effect. The immediate release therapeutically active agent may be incorporated as separate pellets within a gelatin capsule, or may be coated on the surface of the multiparticulates after preparation of the dosage forms such as within a controlled release coating or matrix base. The unit dosage forms of the present invention may also contain a combination of controlled release beads and matrix multiparticulates to achieve a desired effect.

The sustained release formulations of the present invention preferably slowly release the therapeutically active agent, such that when the dosage form is ingested and exposed to gastric fluids, and then to intestinal fluids a therapeutically desirable plasma level is obtained. The sustained release profile of the melt extruded formulations of the invention can be altered, for example, by varying the amount of retardant which may be a hydrophobic material, by varying the amount of plasticizer relative to hydrophobic material, by the inclusion of additional ingredients or excipients, or by altering the method of manufacture, etc.

The melt extruded material can be prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

The following examples illustrate various aspects of the present invention. They are not to be construed to limit the claims in any manner whatsoever.

### Example 1

### Capsule Formulation Containing Gabapentin

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 90.0 mg | 9.00 g |
| Microcrystalline Cellulose | 27.6 mg | 2.76 g |
| Sodium Lauryl Sulfate | 1.6 mg | 0.16 g |
| Total Solid | 210 mg | 21.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 180.0 mg | 18.00 g |
| Microcrystalline Cellulose | 77.4 mg | 7.74 g |
| Sodium Lauryl Sulfate | 1.6 mg | 0.16 g |
| Total Solid | 350 mg | 35.0 g |

Capsule Formulation 3

| | In each | In 100 |
|---|---|---|
| Tramadol Hydrochloride | 35.0 mg | 3.50 g |
| Dextromethorphan Hydrochloride | 45.0 mg | 4.50 g |
| Gabapentin | 90.0 mg | 9.00 g |
| Microcrystalline Cellulose | 36.5 mg | 3.65 g |
| Sodium Lauryl Sulfate | 2.0 mg | 0.20 g |
| Talc | 1.5 mg | 0.15 g |
| Total Solid | 210 mg | 21.0 g |

Capsule Formulation 4

| | In each | In 100 |
|---|---|---|
| Tramadol Hydrochloride | 50.0 mg | 5.00 g |
| Dextromethorphan Hydrochloride | 30.0 mg | 3.00 g |
| Gabapentin | 180.0 mg | 18.00 g |
| Microcrystalline Cellulose | 86.5 mg | 8.65 g |
| Sodium Lauryl Sulfate | 2.0 mg | 0.20 g |
| Talc | 1.5 mg | 0.15 g |
| Total Solid | 350 mg | 35.0 g |

### Example 2

### Capsule Formulation Containing Pregabalin

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Pregabalin | 15.0 mg | 1.50 g |
| Microcrystalline Cellulose | 102.6 mg | 10.26 g |
| Sodium Lauryl Sulfate | 1.6 mg | 0.16 g |
| Total Solid | 210 mg | 21.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Pregabalin | 30.0 mg | 3.00 g |
| Microcrystalline Cellulose | 87.6 mg | 8.76 g |
| Sodium Lauryl Sulfate | 1.6 mg | 0.16 g |
| Total Solid | 210 mg | 21.0 g |

Capsule Formulation 3

| | In each | In 100 |
|---|---|---|
| Tramadol Hydrochloride | 35.0 mg | 3.50 g |
| Dextromethorphan Hydrochloride | 45.0 mg | 4.50 g |
| Pregabalin | 20.0 mg | 2.00 g |
| Microcrystalline Cellulose | 106.5 mg | 10.65 g |
| Sodium Lauryl Sulfate | 2.0 mg | 0.20 g |
| Talc | 1.5 mg | 0.15 g |
| Total Solid | 210 mg | 21.0 g |

Capsule Formulation 4

| | In each | In 100 |
|---|---|---|
| Tramadol Hydrochloride | 50.0 mg | 5.00 g |
| Dextromethorphan Hydrochloride | 30.0 mg | 3.00 g |
| Pregabalin | 40.0 mg | 4.00 g |
| Microcrystalline Cellulose | 86.5 mg | 8.65 g |
| Sodium Lauryl Sulfate | 2.0 mg | 0.20 g |
| Talc | 1.5 mg | 0.15 g |
| Total Solid | 210 mg | 21.0 g |

### Example 3

### Capsule Formulation Containing Amitriptyline

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Amitriptyline Hydrochloride | 11.4 mg | 11.40 g |
| Microcrystalline Cellulose | 106.2 mg | 106.2 g |
| Sodium Lauryl Sulfate | 1.6 mg | 0.16 g |
| Total Solid | 210 mg | 21.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Amitriptyline Hydrochloride | 5.7 mg | 5.70 g |
| Microcrystalline Cellulose | 111.9 mg | 11.19 g |
| Sodium Lauryl Sulfate | 1.6 mg | 0.16 g |
| Total Solid | 210 mg | 21.0 g |

Capsule Formulation 3

| | In each | In 100 |
|---|---|---|
| Tramadol Hydrochloride | 35.0 mg | 3.50 g |
| Dextromethorphan Hydrochloride | 45.0 mg | 4.50 g |
| Amitriptyline Hydrochloride | 11.4 mg | 1.14 g |
| Microcrystalline Cellulose | 115.1 mg | 11.51 g |
| Sodium Lauryl Sulfate | 2.0 mg | 0.20 g |
| Talc | 1.5 mg | 0.15 g |
| Total Solid | 210 mg | 21.0 g |

Capsule Formulation 4

| | In each | In 100 |
|---|---|---|
| Tramadol Hydrochloride | 50.0 mg | 5.00 g |
| Dextromethorphan Hydrochloride | 30.0 mg | 3.00 g |
| Amitriptyline Hydrochloride | 11.4 mg | 1.14 g |
| Microcrystalline Cellulose | 115.1 mg | 11.51 g |
| Sodium Lauryl Sulfate | 2.0 mg | 0.20 g |
| Talc | 1.5 mg | 0.15 g |
| Total Solid | 210 mg | 21.0 g |

### Example 4

### Capsule Formulation Containing Milnacipran

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Milnacipran Hydrochloride | 11.4 mg | 11.40 g |
| Microcrystalline Cellulose | 106.2 mg | 106.2 g |
| Sodium Lauryl Sulfate | 1.6 mg | 0.16 g |
| Total Solid | 210 mg | 21.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Milnacipran Hydrochloride | 5.7 mg | 5.70 g |
| Microcrystalline Cellulose | 111.9 mg | 11.19 g |
| Sodium Lauryl Sulfate | 1.6 mg | 0.16 g |
| Total Solid | 210 mg | 21.0 g |

Capsule Formulation 3

| | In each | In 100 |
|---|---|---|
| Tramadol Hydrochloride | 35.0 mg | 3.50 g |
| Dextromethorphan Hydrochloride | 45.0 mg | 4.50 g |
| Milnacipran Hydrochloride | 11.4 mg | 1.14 g |
| Microcrystalline Cellulose | 115.1 mg | 11.51 g |
| Sodium Lauryl Sulfate | 2.0 mg | 0.20 g |
| Talc | 1.5 mg | 0.15 g |
| Total Solid | 210 mg | 21.0 g |

Capsule Formulation 4

| | In each | In 100 |
|---|---|---|
| Tramadol Hydrochloride | 50.0 mg | 5.00 g |
| Dextromethorphan Hydrochloride | 30.0 mg | 3.00 g |
| Milnacipran Hydrochloride | 11.4 mg | 1.14 g |
| Microcrystalline Cellulose | 115.1 mg | 11.51 g |
| Sodium Lauryl Sulfate | 2.0 mg | 0.20 g |
| Talc | 1.5 mg | 0.15 g |
| Total Solid | 210 mg | 21.0 g |

### Example 5

### Preparation of Capsaicin Palmitate (Formula I, R═CH₃—(CH₂)₁₄)

A mixture of 30.5 gm (˜0.1M) of capsaicin USP27 (HUBEI XIANGXI CHEMICAL INDUSTRY CO., LTD, China), 16.7 ml (0.12M) of anhydrous triethylamine (Spectrum Chemicals), 220 mg of 4-(dimethylamino)pyridine and 200 ml of anhydrous dichloromethane was placed into a 1000 ml 2-neck round bottomed flask. The content was covered with aluminum foil to protect it from light exposure. The flask was fitted with a condenser fitted with a moisture trap on the top and a dropwise addition funnel. The flask was kept at room temperature and 25.4 ml (0.095M) of palmitoyl chloride was added from the funnel into the mixture slowly with stirring. After the addition, the mixture was refluxed for 3-6 hours and stirred for 10-15 hours at room temperature. The mixture was transferred into a separating funnel and washed successively with 2×500 ml of water, 2×500 ml of dilute hydrochloric acid, 2×500 ml of 10% sodium bicarbonate solution and 3×500 ml of type I water. The organic layer was separated, dried with anhydrous magnesium sulfate and the dichloromethane was removed under vacuum to produce a light yellow solid (95% of theoretical). The light yellow solid, as obtained above, was re-crystallized from ethanol. In a 2-liter flask, the solid was dissolved in 1 liter of hot ethanol and filtered through a filter paper. The filtrate was then cooled in the refrigerator to get white crystals.

### Example 6

### Capsule Formulation Containing Gabapentin and Capsaicin Palmitate

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 90.0 mg | 9.00 g |
| Capsaicin palmitate | 5.4 mg | 0.54 g |
| Microcrystalline Cellulose | 32.2 mg | 3.22 g |
| Sodium Lauryl Sulfate | 1.6 mg | 0.16 g |
| Total Solid | 220 mg | 22.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 180.0 mg | 18.00 g |
| Capsaicin palmitate | 5.4 mg | 0.54 g |
| Microcrystalline Cellulose | 72.4 mg | 7.24 g |
| Sodium Lauryl Sulfate | 1.6 mg | 0.16 g |
| Total Solid | 350 mg | 35.0 g |

Capsule Formulation 3

| | In each | In 100 |
|---|---|---|
| Tramadol Hydrochloride | 35.0 mg | 3.50 g |
| Dextromethorphan Hydrochloride | 45.0 mg | 4.50 g |
| Gabapentin | 90.0 mg | 9.00 g |
| Capsaicin palmitate | 10.8 mg | 1.08 g |
| Microcrystalline Cellulose | 35.7 mg | 3.57 g |
| Sodium Lauryl Sulfate | 2.0 mg | 0.20 g |
| Talc | 1.5 mg | 0.15 g |
| Total Solid | 220 mg | 22.0 g |

Capsule Formulation 4

| | In each | In 100 |
|---|---|---|
| Tramadol Hydrochloride | 50.0 mg | 5.00 g |
| Dextromethorphan Hydrochloride | 30.0 mg | 3.00 g |
| Gabapentin | 180.0 mg | 18.00 g |
| Capsaicin palmitate | 5.4 mg | 0.54 g |
| Microcrystalline Cellulose | 81.1 mg | 8.11 g |
| Sodium Lauryl Sulfate | 2.0 mg | 0.20 g |
| Talc | 1.5 mg | 0.15 g |
| Total Solid | 350 mg | 35.0 g |

### Example 7

### Capsule Formulation Containing Gabapentin and Milnacipran

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 45.0 mg | 9.00 g |
| Milnacipran Hydrochloride | 5.7 mg | 0.57 g |
| Microcrystalline Cellulose | 66.9 mg | 6.69 g |
| Sodium Lauryl Sulfate | 1.6 mg | 0.16 g |
| Total Solid | 210 mg | 21.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 90.0 mg | 18.00 g |
| Milnacipran Hydrochloride | 5.7 mg | 0.57 g |
| Microcrystalline Cellulose | 31.9 mg | 3.19 g |
| Sodium Lauryl Sulfate | 1.6 mg | 0.16 g |
| Total Solid | 220 mg | 35.0 g |

Capsule Formulation 3

| | In each | In 100 |
|---|---|---|
| Tramadol Hydrochloride | 35.0 mg | 3.50 g |
| Dextromethorphan Hydrochloride | 45.0 mg | 4.50 g |
| Gabapentin | 90.0 mg | 9.00 g |
| Milnacipran Hydrochloride | 11.4 mg | 1.14 g |
| Microcrystalline Cellulose | 35.1 mg | 3.51 g |
| Sodium Lauryl Sulfate | 2.0 mg | 0.20 g |
| Talc | 1.5 mg | 0.15 g |
| Total Solid | 220 mg | 22.0 g |

Capsule Formulation 4

| | In each | In 100 |
|---|---|---|
| Tramadol Hydrochloride | 50.0 mg | 5.00 g |
| Dextromethorphan Hydrochloride | 30.0 mg | 3.00 g |
| Gabapentin | 180.0 mg | 18.00 g |
| Milnacipran Hydrochloride | 5.7 mg | 0.57 g |
| Microcrystalline Cellulose | 80.8 mg | 8.08 g |
| Sodium Lauryl Sulfate | 2.0 mg | 0.20 g |
| Talc | 1.5 mg | 0.15 g |
| Total Solid | 350 mg | 35.0 g |

### Example 8

### Capsule Formulation Containing Gabapentin and Amitriptyline

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 45.0 mg | 9.00 g |
| Amitriptyline Hydrochloride | 5.7 mg | 0.57 g |
| Microcrystalline Cellulose | 66.9 mg | 6.69 g |
| Sodium Lauryl Sulfate | 1.6 mg | 0.16 g |
| Total Solid | 210 mg | 21.0 g |

Capsule Formulation 2

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 90.0 mg | 18.00 g |
| Amitriptyline Hydrochloride | 5.7 mg | 0.57 g |
| Microcrystalline Cellulose | 31.9 mg | 3.19 g |
| Sodium Lauryl Sulfate | 1.6 mg | 0.16 g |
| Total Solid | 220 mg | 35.0 g |

Capsule Formulation 3

| | In each | In 100 |
|---|---|---|
| Tramadol Hydrochloride | 28.5 mg | 2.85 g |
| Dextromethorphan Hydrochloride | 34.0 mg | 3.40 g |
| Gabapentin | 30.0 mg | 3.00 g |
| Amitriptyline Hydrochloride | 5.7 mg | 0.57 g |
| Microcrystalline Cellulose | 118.3 mg | 11.83 g |
| Sodium Lauryl Sulfate | 2.0 mg | 0.20 g |
| Talc | 1.5 mg | 0.15 g |
| Total Solid | 220 mg | 22.0 g |

### Example 9

### Capsule Formulation Containing Gabapentin, Amitriptyline and Capsaicin Palmitate

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 39.8 mg | 3.98 g |
| Dextromethorphan Hydrochloride | 51.0 mg | 5.10 g |
| Gabapentin | 30.0 mg | 3.00 g |
| Amitriptyline Hydrochloride | 5.7 mg | 0.57 g |
| Capsaicin palmitate | 5.4 mg | 0.54 g |
| Microcrystalline Cellulose | 86.5 mg | 8.65 g |
| Sodium Lauryl Sulfate | 1.6 mg | 0.16 g |
| Total Solid | 220 mg | 22.0 g |

Capsule Formulation 2

| | In each | In 100 |
|---|---|---|
| Tramadol Hydrochloride | 28.5 mg | 2.85 g |
| Dextromethorphan Hydrochloride | 34.0 mg | 3.40 g |
| Gabapentin | 30.0 mg | 3.00 g |
| Amitriptyline Hydrochloride | 5.7 mg | 0.57 g |
| Capsaicin palmitate | 5.4 mg | 0.54 g |
| Microcrystalline Cellulose | 114.8 mg | 11.48 g |
| Sodium Lauryl Sulfate | 1.6 mg | 0.16 g |
| Total Solid | 350 mg | 35.0 g |

### Example 10

### Capsule Formulation Containing Magnesium

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

Capsule Formulation 1

| | In each | In 100 |
|---|---|---|
| Tramadol Hydrochloride | 50.0 mg | 5.00 g |
| Magnesium Sulfate anhydrous | 100.0 mg | 10.00 g |
| Gabapentin | 100.0 mg | 10.00 g |
| Microcrystalline Cellulose | 3.9 mg | 0.39 g |
| Sodium Lauryl Sulfate | 1.0 mg | 0.10 g |
| Silicon Dioxide | 4.1 mg | 0.41 g |
| Magnesium Stearate | 1.0 mg | 0.10 g |
| Total Solid | 260 mg | 26.0 g |

Capsule Formulation 2

| | In each | In 100 |
|---|---|---|
| Tramadol Hydrochloride | 50.0 mg | 5.00 g |
| Magnesium Sulfate anhydrous | 100.0 mg | 10.00 g |
| Gabapentin | 100.0 mg | 10.00 g |
| Capsaicin Palmitate | 5.4 mg | 0.54 g |
| Microcrystalline Cellulose | 3.9 mg | 0.39 g |
| Sodium Lauryl Sulfate | 1.0 mg | 0.10 g |
| Silicon Dioxide | 4.1 mg | 0.41 g |
| Magnesium Stearate | 1.0 mg | 0.10 g |
| Total Solid | 265.4 mg | 26.54 g |

### Example 11

### The Effect of the Combination Therapy Containing Magnesium in Humans

### Patient 1: Condition: Bone Spur

A 55 year old white female had constant pain due to bone spur next to the Achilles. She was provided with TLI-0326, the code name for the formulation of 1 in Example 10. She wrote the following testimonial.

“Having a bone spur next to the Achilles heel has been rather painful. Surgery has been suggested but can't be done at this time.

The 0326 has been a Godsend. Having to be on my feet at work used to be very painful. Now I can walk in comfort.

I feel the product is good for at least 12 hours, sometimes longer, depending on how stressful the day has been. Also has not been a problem mixing with my other meds or having any ill effects on my one sickness”.

### Patient 2: Condition: Diabetic Neuropathy

A 52 year old white male is suffering from diabetic neuropathy and he was taking TLI-1026 which is the formulation 1 in Example 1. He was switched to TLI-0326, the code name for the formulation of 1 in Example 10. He wrote the following. “I suffer from pain caused by diabetes. Neuropathic pain is extremely painful and can keep you up all night. It moves into your hands and causes other problems. I've taken the narcotic lines and they only take the edge off the pain for a couple hours. For the past 3 years I was taking 3 capsules of TLI-1026 every day. I was given the new formula called TLI-0326. The TIL-0326 takes about 30 minutes to take effect on the first dose. If you are going for long term use take 3 a day or less as needed. There were no side effects at all with other medication, Thank God for this invention”

### Patient 3: Condition: Fibromyalgia

A 44 year old white female has fibromyalgia and she was given TLI-1026 through one of her family friend. He wrote the following. “Through the Alzafar Shrine in San Antonio, Tex. I met a lodge brother Les Radcliffe. Les related to me one day that his wife was not feeling well and could not be at one of the Rod and Gun Club meeting my company caters for them, Time after time she was noticeably absent from our meetings. With tears in his eyes, he told me of her tremendous pain and there was nothing he could do to help her. I told him of the TIL 0326 pain relief I had experienced and he asked if there was any possibility it could have the same effect for his wife that I had experienced.

The week after he made the request to his wife took the drug. I sat with her for one and a half hours after she ingested the drug. In 68 minutes after she took the medication she rubbed below her knee and above her ankle and said “The fibromyalgia pain I have is gone. I can touch my leg . . . Look.” The woman is on a huge list of very serious medications and none, for pain, seen to be effective. The TIL 0326 worked within one hour on her”

### Patient 4: Condition: Dental Pain

A 50 year old white male was suffering from pain and he was provided with TLI-0326 and he gave the following testimonial. “I of my own free will and accord, do here testify to the effectiveness of the drug known as TLI 0326. Upon biting down on my favorite meat lovers' pizza at a nearby Italian sandwich ship and I broke a tooth. There was a little pain in the time following the fracture. As time passed the tooth let me know there would be a need for attention. As time passed further, the pain was significant. Busy in my catering business, I let time pass once again. There was a point on this timeline, in which the pain became unbearable; not only interrupting my sleep but fully let me now I was in trouble.

I called my dentist in San Antonio, Tex. to get pain medication. He answered the phone and informed me he was on a skiing trip in Colorado and would not be back in San Antonio for four days! This was absolutely a situation I found I had no way out of. Sharing this information with my close friend and informing him of my situation, he told me of the research being done on pain medication TLI-0326. He told me its nature, side effects and effectiveness. Doug told me how the drug worked and that I, because of my unbearable pain might want to use the medication. The first night (I used the medication) I slept for the first time in two days.

The next three days were a wondrous adventure of pain freedom.

The four day reprieve from pain allowed me sleep and to awaken without the “drug head” side effect present in most heavy pain killer drugs.

Upon visiting the dentist he informed me the tooth would have to be removed but only after a ten to two week saturation of antibiotics. He offered me a prescription for pain medication and two types of antibiotics. The pain medication was not necessary because I had witnessed firsthand the effectiveness of TLI-0326. The antibiotics, after two weeks, reduced the golf ball sized abscess and the TLI 0326 kept me comfortable until my tooth was safely removed.

There was again an opportunity to have prescribed pain medication by my dentist. This again was not necessary because of the effectiveness of TLI-0326.

I can tell you, from my experience, there was no down side from this drug. The up side effect is that the intense pain stopped in short order and there is no hang over feeling any of the mornings following its use.

Would I again reach for this substance? I would use TLI-0326 without reservation because within one hour from starting the drug, pain is noticeably gone. All it takes to float it away is take additional dosage”.

### Patient 5: Condition: Diabetic Neuropathy

A 55 year old white female who is working in laundry store is suffering from diabetic neuropathy. Her physician used to inject steroids in the leg to control her pain. She was given to TLI-0326, the code name for the formulation of 1 in Example 10. She told the following. “I suffer from pain caused by diabetes and my foot used to hurt all the time. I have taken the narcotics and steroid shots in the foot and they only take the edge off the pain for a few hours. Due to steroids, I used to have other problems. For the past 9 months I am taking 2 capsules of TLI-0326 every day. The TLI-0326 takes about 30 minutes to take effect on the first dose. At night I can sleep very well and in the morning there is no side effect associated with other drugs”

Patient 5: Condition: Neuropathy associated with Chemotherapy

A 45 year old female dentist had breast cancer and undergone mastectomy to remove the tumor and had undergone additional chemotherapy. Due to the chemotherapy, she developed permanent neuropathy and used to control pain by yoga and other natural treatment. She was given TLI-0326 and was taking 2 capsules per day. She said the capsule worked fine for her and could sleep at night comfortably. However, she wished permanent cure for her neuropathy as TLI-0326 should be taken every day to contain her pain.

### Patient 6: Condition: Back Pain

A 72 year old male dentist have constant back pain. He wanted to try TLI-0326 and was taking 2 capsules per day. He said the capsule worked extremely well in relieving his pain and could sleep at night comfortably. He is taking TLI-0326 whenever he needs relief for his back.

It is, therefore, apparent that there has been provided, in accordance with the present invention, compositions for treating pain. Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims.

1. A pharmaceutical composition comprising a combination of:
a) tramadol or a pharmaceutically acceptable salt thereof;
b) magnesium or a pharmaceutically acceptable salt thereof; and
c) gabapentin or pregabalin; and
wherein said tramadol, said gabapentin or pregabalin, and said magnesium are in a normal or immediate release formulation.

2. The pharmaceutical composition of claim 1, wherein the composition comprises tramadol, gabapentin, and magnesium as the only active ingredients, wherein a weight ratio between magnesium, tramadol and gabapentin is 1.0:0.5-4.0:1.0-8.0.

3. The pharmaceutical composition of claim 2, wherein the tramadol is present at a dose of from about 30 mg to about 60 mg; and wherein the magnesium is present at a dose of from about 20 mg to about 120 mg; and wherein the gabapentin is present at a dose of from about 30 mg to about 150 mg.

4. The pharmaceutical composition of claim 6, wherein the composition comprises from about 28 mg to about 50 mg of tramadol, from about 30 mg to about 100 mg of magnesium, and from about 60 mg to about 120 mg of gabapentin.

5. The pharmaceutical composition of claim 2, wherein the amount of any one of the tramadol, magnesium, or gabapentin would be sub-therapeutic when administered without the other two agents.

6. The pharmaceutical composition of claim 1, wherein the pharmaceutical composition is formulated for oral administration, a solution, a suspension or elixir for oral administration, an injectable formulation, comprised in an implantable device, a topical preparation, comprised in a solid state or depot type transdermal delivery device, a suppository, a buccal tablet, or an inhalation formulation.

7. The pharmaceutical composition of claim 6, wherein the pharmaceutical composition is formulated for oral administration as a tablet or encapsulated multiparticulate formulation.

8. The pharmaceutical composition of claim 1, wherein the composition is free or essentially free of a NSAID or acetaminophen.

9. The pharmaceutical composition of claim 1, wherein the composition further comprises capsaicin palmitate.

10. A method of treating pain in a subject, comprising administering to a subject an amount of agents including:
a) tramadol or a pharmaceutically acceptable salt thereof;
b) magnesium or a pharmaceutically acceptable salt thereof; and
c) gabapentin or pregabalin; and
wherein said tramadol, said gabapentin or pregabalin, and said magnesium are in a normal or immediate release formulation; and wherein the combined amount of said agents is effective to treat pain.

11. The method of claim 10, wherein the pain results from fibromyalgia syndrome, diabetic neuropathy syndrome, multiple sclerosis, post-surgery pain, arachnoiditis syndrome, trigeminal neuralgia, post-herpetic neuralgia, a cancer, chemotherapy or a radiation therapy.

12. The method of claim 10, wherein the method comprises administering the agents simultaneously or essentially simultaneously.

13. The method of claim 12, wherein the agents are administered in a single formulation.

14. The method of claim 10, wherein the method comprises administering the agents sequentially.

15. (canceled)

16. The method of claim 10, wherein the agents are administered orally, intravenously, by means of an implant, parenterally, sub-dermally, sublingually, rectally, topically, or via inhalation.

17. The method of claim 16, wherein the agents are administered orally in a tablet or capsule formulation.

18. The method of claim 10, wherein the subject is a human.

19. The method of claim 18, wherein the combined amount of the agents results in little or no toxicity to the subject.

20. (canceled)

21. (canceled)

22. A method of reducing the amount of tramadol required to treat a patient affected with pain, comprising further administering to a patient being treated with tramadol or a pharmaceutically acceptable salt thereof, an amount of
a) magnesium or a pharmaceutically acceptable salt thereof; and
b) gabapentin or pregabalin; and
wherein the combination of a) and b) are effective to augment the analgesia attributable to said tramadol during at least a portion of the dosage interval of said tramadol.

23. (canceled)

24. (canceled)

25. The method of claim 22, further comprising administering a capsaicin palmitate to the subject.
